(19) United States
(12) Patent Application Publication
(10) Pub. No.: US 2026/0305542 A1
(43) Pub. Date: Oct. 8, 2026

(54) FUNGUS CULTIVATION USING ALGAL BIOMASS AS SUBSTRATES AND METHODS OF USE THEREOF

(71) Applicant: UNIVERSITY OF HOUSTON SYSTEM, Houston, TX (US)

(72) Inventors: Venkatesh BALAN, Sugarland, TX (US); Mahsa ALIAN, Houston, TX (US)

(73) Assignee: UNIVERSITY OF HOUSTON SYSTEM, Houston, TX (US)

(21) Appl. No.: 19/482,868

(22) PCT Filed: May 31, 2024
(86) PCT No.: PCT/US2024/031863

§ 371 Date: Nov. 10, 2025

Related U.S. Application Data
(60) Provisional application No. 63/470,732, filed on Jun. 2, 2023.

(51) Int. Cl. A01G 18/20; A01G 33/00; C12N 1/12

(52) U.S. Cl. CPC A01G 18/20; A01G 33/00; C12N 1/12

(57) ABSTRACT

The present invention relates to cultivating fungus and to materials and/or methods for producing, growing, and/or cultivating fungus.

### CROSS-REFERENCE TO RELATED APPLICATIONS

This application is a national phase of International Application No. PCT/US2024/031863 filed May 31, 2024, which designated the U.S. and that International Application was published under PCT Article 21(2) in English, and this application also claims the benefit of priority under 35 U.S.C. § 119(e) to U.S. Provisional Patent Application No. 63/470,732 filed Jun. 2, 2023, the contents of each of which are incorporated herein by reference in their entirety.

### STATEMENT REGARDING FEDERALLY SPONSORED RESEARCH OR DEVELOPMENT

This invention was made with government support under Grant No. 13111855 awarded by the United States Department of Agriculture. The government has certain rights in the invention.

### FIELD OF THE INVENTION

The present invention relates to cultivating fungus and to materials and/or methods for producing, growing, and/or cultivating fungus.

### BACKGROUND

The increasing demand for sustainable and eco-friendly agricultural practices has spurred interest in alternative substrates for mushroom cultivation. Lignocellulosic biomass and grains have been the traditional substrate of choice for producing mycelium and mushrooms. Fungal mycelium produced using lignocellulosic biomass and grains is widely used by Biotech companies to produce many enzymes and sustainable materials. Mycelium products include biodegradable packing materials foam (Ecovative, NY), concrete, bioplastic, leather (Mycoworks, CA), textiles (Mylum, Netherlands), alternative meat products (Beyond Meat Inc, CA), food products (Atlast Food, NY) and other supplements (Aloha, NV). It is estimated that the global mycelium market will grow to $222.3 million by 2027. Currently, mycelium producers are using grains or lignocellulosic biomass as substrates.

Thus, there is a need in the art for improved materials and methods for producing, growing, and/or cultivating fungus, mushrooms, and/or mycelium. The present disclosure address these and other unfulfilled needs in the art.

### SUMMARY OF THE INVENTION

In various embodiments, the present invention provides a substrate for cultivating at least one fungal mycelium or at least one mushroom, the substrate comprising: at least one algae, provided that the at least one algae is not Spirulina maxima or Chlorella vulgaris. In some embodiments, the at least one algae has been treated to remove at least a portion of salt from the at least one algae. In some embodiments, the substrate further comprises lignocellulosic biomass. In some embodiments, the at least one algae is at least one microalgae, or at least one macroalgae, or combination thereof, provided that the at least one microalgae is not Spirulina maxima or Chlorella vulgaris. In some embodiments, the at least one macroalgae is nori, bladderwrack, Irish moss, kelp, dulse, or wakame, or any combination thereof. In some embodiments, the at least one fungal mycelium or the at least one mushroom is Hypsizygus ulmarius, Calocybe indica, Pleurotus ostreatus, Ganoderma lucidum, Agaricus bisporus, Ganoderma lucidum, Grifola frondosa, Fomes fomentarius, Daedaleopsis confragosa, Lentinula edodes, Pleurotus ostreatus, Agrocybe aegerita, Hericium erinaceus, or Morchella angusticeps, or any combination thereof.

In various embodiments, the present invention provides a method for cultivating at least one fungal mycelium or at least one mushroom, the method comprising: providing a substrate of the present invention; inoculating the substrate with fungal spores or fungal mycelium; and applying conditions to stimulate growth of at least one fungal mycelium or at least one mushroom. In some embodiments, the substrate further comprises lignocellulosic biomass. In some embodiments, the at least one algae is at least one microalgae, or at least one macroalgae, or combination thereof, provided that the at least one microalgae is not Spirulina maxima or Chlorella vulgaris. In some embodiments, the at least one macroalgae is nori, bladderwrack, Irish moss, kelp, dulse, or wakame, or any combination thereof.

In various embodiments, the present invention provides a method for cultivating at least one fungal mycelium or at least one mushroom and for producing algal biomass, the method comprising: providing a substrate of the present invention; inoculating the substrate with fungal spores or fungal mycelium; applying conditions to stimulate growth of at least one fungal mycelium or at least one mushroom; capturing carbon dioxide produced during the cultivation of the at least one fungal mycelium or the at least one mushroom; converting the captured carbon dioxide into sodium bicarbonate; and utilizing the sodium bicarbonate as a carbon source to produce algal biomass. In some embodiments, the substrate further comprises lignocellulosic biomass. In some embodiments, the at least one algae is at least one microalgae, or at least one macroalgae, or combination thereof, provided that the at least one microalgae is not Spirulina maxima or Chlorella vulgaris. In some embodiments, the at least one macroalgae is nori, bladderwrack, Irish moss, kelp, dulse, or wakame, or any combination thereof.

In various embodiments, the present invention provides a system for cultivating at least one fungal mycelium or at least one mushroom and for producing algal biomass, the system comprising: a first chamber, wherein the first chamber is adapted for cultivating at least one fungal mycelium or at least one mushroom and capturing carbon dioxide produced during cultivation of the at least one fungal mycelium or the at least one mushroom, and wherein the first chamber comprises a substrate of the present invention; a second chamber, wherein the second chamber is in communication with the first chamber, and wherein the second chamber is adapted to convert the captured carbon dioxide into sodium bicarbonate; and a third chamber, wherein the third chamber is in communication with the second chamber, and wherein the third chamber is adapted for using the sodium bicarbonate as a carbon source for producing algal biomass. In some embodiments, the substrate further comprises lignocellulosic biomass. In some embodiments, the at least one algae is at least one microalgae, or at least one macroalgae, or combination thereof, provided that the at least one microalgae is not Spirulina maxima or Chlorella vulgaris. In some embodiments, the at least one macroalgae is nori, bladderwrack, Irish moss, kelp, dulse, or wakame, or any combination thereof.

### BRIEF DESCRIPTION OF THE DRAWINGS

FIG. 1 depicts in accordance with various embodiments of the invention, Growth rate of Calocybe indica mycelium when grown on LB containing media. Here, PDA Ctrl: Potato dextrose agar control, Agar Ctrl: Simple agar control, LB: lignocellulosic biomass, 4^{th}-day culture plates and 7^{th}-day culture plates.

FIG. 2 depicts in accordance with various embodiments of the invention, Growth rate of Calocybe indica mycelium when grown on chlorella containing media. Here, PDA Ctrl: Potato dextrose agar control, Agar Ctrl: Simple agar control, 4^{th}-day culture plates and 7^{th}-day culture plates.

FIG. 3 depicts in accordance with various embodiments of the invention, Growth rate of Calocybe indica mycelium when grown on spirulina containing media. PDA Ctrl: Potato dextrose agar control, Agar Ctrl: Simple agar control, 4^{th}-day culture plates and 7^{th} day culture plates.

FIG. 4 depicts in accordance with various embodiments of the invention, Growth rate of Calocybe indica mycelium when grown on the combination of chlorella and spirulina-containing media at various solids loading. Here, PDA Ctrl: Potato dextrose agar control, Agar Ctrl: Simple agar control, 4^{th}-day culture plates and 7^{th}-day culture plates.

FIG. 5A-FIG. 5B depicts in accordance with various embodiments of the invention, Wet weight (FIG. 5A) and dry weight (FIG. 5B) of Calocybe indica mycelium. Here, PDA: Potato Dextrose Agar, Agar Ctrl: Simple agar control, Ch: chlorella, Sp: Spirulina, Com: Combination of chlorella and spirulina (1:1) in the media.

FIG. 6 depicts in accordance with various embodiments of the invention, Growth rate of Calocybe indica mycelium when grown on chlorella, spirulina, and their combination (1:1) algae media with PDA agar. Here, PDA Ctrl: Potato dextrose agar control, Agar Ctrl: Simple agar control, Ch: Chlorella, Sp: Spirulina, Com: Combination of chlorella and spirulina (1:1), 4^{th}-day culture plates and 7th-day culture plates.

FIG. 7A-FIG. 7B depicts in accordance with various embodiments of the invention, Wet weight (FIG. 7A) and dry weight (FIG. 7B) of Calocybe indica mycelium when grown on chlorella, spirulina, and their combination (1:1) in PDA agar-containing media. Here, PDA Ctrl: Potato dextrose agar control, Ch: Chlorella, Sp: Spirulina, Com: Combination of chlorella and spirulina (1:1).

FIG. 8 depicts in accordance with various embodiments of the invention, Relative increase in wet and dry weight of the Calocybe indica mycelium when grown on different algae-containing media with PDA compared to the PDA control. Here, Ch: Chlorella, Sp: Spirulina, Com: Combination of chlorella and spirulina (1:1).

FIG. 9 depicts in accordance with various embodiments of the invention, Growth rate of Calocybe indica mycelium when grown on different macroalgae containing media with PDA. Here, PDA: Potato Dextrose Agar, Agar Ctrl: Simple agar control, LB: lignocellulosic biomass, No: Nori, Bl: Bladderwrack, IM: Irish Moss, KE: Kelp extract, Du: Dulse, 4^{th} day culture plates and 7^{th} day culture plates.

FIG. 10A-FIG. 10B depicts in accordance with various embodiments of the invention, Wet weight (FIG. 10A) and dry weight (FIG. 10B) of Calocybe indica mycelium when grown on macroalgae media with PDA. Here, PDA: Potato Dextrose Agar, Agar Ctrl: Simple agar control, LB: lignocellulosic biomass, No: Nori, Bl Bladderwrack, IM: Irish Moss, KE: Kelp extract and Du: Dulse.

FIG. 11 depicts in accordance with various embodiments of the invention, Growth rate of Calocybe indica mycelium when grown on different macroalgae containing media with agar. Here, PDA: Potato Dextrose Agar, Agar Ctrl: Simple agar control, LB: lignocellulosic biomass, No: Nori, Bl: Bladderwrack, IM: Irish Moss and KE: Kelp extract, Du: Dulse, Wa: Wakame, 4^{th}-day culture plates and 7th-day culture plates.

FIG. 12A-FIG. 12B depicts in accordance with various embodiments of the invention, Growth rate of Calocybe indica mycelium when grown on macroalgae containing media with agar. Here, PDA: Potato Dextrose Agar, Agar Ctrl: Simple agar control, LB: lignocellulosic biomass, No: Nori, Bl: Bladderwrack, IM: Irish Moss, KE: Kelp extract and Du: Dulse and Wa: Wakame.

FIG. 13 depicts in accordance with various embodiments of the invention, Growth rate of Calocybe indica mycelium when grown on the combination of macroalgae and lignocellulosic biomass (1:1) containing media with agar. Here, PDA: Potato Dextrose Agar, Agar Ctrl: Simple agar control, LB: lignocellulosic biomass, No: Nori, Bl: Bladderwrack, IM: Irish Moss, KE: Kelp extract, Du: Dulse and Wa: Wakame, 4th-day culture plates and 7th-day culture plates.

FIG. 14A-FIG. 14B depicts in accordance with various embodiments of the invention, Wet weight (FIG. 14A) and dry weight (FIG. 14B) of Calocybe indica mycelium when grown on a combination of macroalgae and lignocellulosic biomass (1:1) containing media with agar. Here, PDA: Potato Dextrose Agar, Agar Ctrl: Simple agar control, LB: lignocellulosic biomass, No: Nori, Bl: Bladderwrack, IM: Irish Moss, KE: Kelp extract, Du: Dulse, Wa: Wakame.

FIG. 15 depicts in accordance with various embodiments of the invention, Relative increase in wet and dry weight of the Calocybe indica mycelium when grown on different algae-containing media compared to the PDA control. Here, PDA: Potato Dextrose Agar, Agar Ctrl: Simple agar control, LB: lignocellulosic biomass, No: Nori, Bl: Bladderwrack, IM: Irish Moss, KE: Kelp extract, Du: Dulse and Wa: Wakame.

FIG. 16 depicts in accordance with various embodiments of the invention, Effect of macro and microalgae on the mycelium growth of Hypsizygus ulmarius (HU). Lignocellulosic biomass (LB) was used as a control substrate. Macroalgae and microalgae were used as alternative substrates, either alone or in combination with LB. PDA: Potato Dextrose Agar, Ch: Chlorella, Sp: Spirulina, No: Nori, Du: Dulse.

FIG. 17 depicts in accordance with various embodiments of the invention, Effect of macro and microalgae on the mycelium growth of Calocybe indica (CI). Lignocellulosic biomass (LB) was used as a control substrate. Macroalgae and microalgae were used as alternative substrates, either alone or in combination with LB. Here, PDA: Potato Dextrose Agar, Ch: Chlorella, Sp: Spirulina, No: Nori, Du: Dulse.

FIG. 18 depicts in accordance with various embodiments of the invention, Effect of macro and microalgae on the mycelium growth of Pleurotus ostreatus (PO). Lignocellulosic biomass (LB) was used as a control substrate. Macroalgae and microalgae were used as alternative substrates, either alone or in combination with LB. Here, PDA: Potato Dextrose Agar, Ch: Chlorella, Sp: Spirulina, No: Nori, Du: Dulse.

FIG. 19 depicts in accordance with various embodiments of the invention, Effect of macro and microalgae on the mycelium growth of Ganoderma lucidum (GL). Lignocellulosic biomass (LB) was used as a control substrate. Macroalgae and microalgae were used as alternative substrates, either alone or in combination with LB. Here, PDA: Potato Dextrose Agar, Ch: Chlorella, Sp: Spirulina, No: Nori, Du: Dulse.

FIG. 20 depicts in accordance with various embodiments of the invention, Effect of macro and microalgae on the mycelium growth of Agaricus bisporus. Composted Lignocellulosic biomass (CLB) was used as a control substrate. Macroalgae and microalgae were used as alternative substrates, either alone or in combination with CLB. Here, PDA: Potato Dextrose Agar, Ch: Chlorella, Sp: Spirulina, No: Nori, Du: Dulse.

FIG. 21 depicts in accordance with various embodiments of the invention, Effect of macroalgae on actual substrate on the mycelium growth and fruiting body formation of Hypsizygus ulmarius. Pure macroalgae substrates, such as sea moss (Mo) and sea lettuce (Le), Kombu Kelp (Ko), were compared with a combination of macroalgae and lignocellulosic biomass (MA+LB) and pure lignocellulosic biomass (LB). The time required for full colonization and fruiting body formation was recorded.

FIG. 22A-FIG. 22B depicts in accordance with various embodiments of the invention, Schematic representation of the current method (FIG. 22A) and proposed method (FIG. 22B) for producing mycelium/mushrooms. The proposed closed-loop system (FIG. 22B) integrates CO₂ capture from mushroom/mycelium cultivation, conversion of CO₂ to NaHCO₃, algal biomass production using NaHCO₃, and utilization of algal biomass as a substrate for mushroom/mycelium production. The spent mushroom substrate (SMS) is used as animal feed.

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs.

One skilled in the art will recognize many methods and materials similar or equivalent to those described herein, which could be used in the practice of the present invention. Other features and advantages of the invention will become apparent from the following detailed description, taken in conjunction with the accompanying drawings, which illustrate, by way of example, various features of embodiments of the invention. Indeed, the present invention is in no way limited to the methods and materials described. For convenience, certain terms employed herein, in the specification, examples and appended claims are collected here.

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. Unless otherwise defined, all technical and scientific terms used herein have the same meaning as commonly understood by one of ordinary skill in the art to which this invention belongs. This invention is not limited to the particular methodology, protocols, and reagents, etc., described herein and as such can vary. The definitions and terminology used herein are provided to aid in describing various embodiments, and are not intended to limit the claimed invention, because the scope of the invention is limited only by the claims.

As used herein the term “comprising” or “comprises” is used in reference to compositions, methods, systems, articles of manufacture, apparatus, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

Unless stated otherwise, the terms “a” and “an” and “the” and similar references used in the context of describing a particular embodiment of the application (especially in the context of claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of all examples, or exemplary language (for example, “such as”) provided with respect to certain embodiments herein is intended merely to better illuminate the application and does not pose a limitation on the scope of the application otherwise claimed. The abbreviation, “e.g.,” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

“Optional” or “optionally” means that the subsequently described circumstance may or may not occur, so that the description includes instances where the circumstance occurs and instances where it does not.

In some embodiments, the numbers expressing quantities of reagents, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed considering the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Here, we explore the use of micro- and macroalgae as a potential substrate due to their high nutritional content and ability to capture carbon dioxide (CO₂) during their own life cycle. Using algae as a substrate for mushroom cultivation can provide a range of benefits. (1) microalgae can be produced in large quantities using cost-effective methods using raceway pond and photo bioreactors and macroalgae can be produced in the ocean. (2) Mycelium-based materials are typically produced from agricultural waste, which can be subject to supply chain issues; such as seasonality and availability; using seaweeds as substrate will help to overcome the problem ensuring a consistent and reliable supply of mycelium year around. (3) Algae can provide a rich source of nutrients for mushroom growth, including proteins, carbohydrates, and lipids, and can be broken down into simpler compounds by mycelium to be used as a food source. (4) Ability to produce carbon free mycelium and mushrooms since algae is a phototropic organism that uses CO₂ from the atmosphere as carbon source and build their own body mass. In other words, using algae as a substrate can help to reduce greenhouse gas (GHG) emissions associated with mushroom farming.

We have discovered different micro- and macroalgae have unique nutrient profiles that can support mycelium growth. For example, some species of algae are rich in essential amino acids like lysine and tryptophan, which are necessary for mycelium growth. Other species of algae are high in vitamins and minerals like iron, calcium, and phosphorus, which can promote healthy mycelial growth. Furthermore, algae-based substrates can contain natural growth-promoting compounds, such as hormones and growth regulators, which can stimulate mycelial growth. These compounds can help to enhance the growth and development of the mycelium, leading to denser and more robust fungal networks in a shorter period. In addition to promoting mycelial growth, using algae-based substrates in mushroom cultivation can also reduce the environmental impact of mushroom farming by capturing and utilizing CO₂ produced during the cultivation process. During mushroom cultivation, CO₂ is produced as a byproduct of respiration by both the mycelium and the mushrooms themselves. Traditionally, this CO₂ is vented out of the growing environment and released into the atmosphere, contributing to GHG emissions and climate change. However, using algae-based substrates provides a sustainable and environmentally friendly way to capture and utilize this CO₂. Algae are photosynthetic organisms that can use CO₂ as a source of carbon for growth. By providing algae with the CO₂ produced during mushroom cultivation, farmers can reduce their environmental impact while also producing a valuable source of nutrients for mycelium growth. Once the algae have grown, they can be harvested and used as a nutrient source for mycelium growth, as described earlier. This closed-loop system, where CO₂ produced during mushroom cultivation is captured and used to produce algae, which is then used to grow mushrooms, represents a sustainable and circular approach to agriculture that can help to reduce GHG emissions and promote sustainability. Furthermore, using algae-based substrates can help to reduce the use of synthetic fertilizers and other chemical inputs in mushroom cultivation, further reducing the environmental impact of this industry. By using natural, renewable sources of nutrients like algae, farmers can promote a more sustainable and environmentally friendly approach to mushroom cultivation.

Nutrient diversity: Using micro and macroalgae or a combination of algae and lignocellulosic biomass as substrate for mycelium and mushroom cultivation has the potential to offer more nutritional benefits compared to using lignocellulosic biomass or grains alone. Algae have high levels of proteins, lipids, and carbohydrates, making them a diverse and rich source of nutrients for mushroom growth. Combining algae with lignocellulosic biomass can also help maintain an optimal carbon-to-nitrogen ratio in the substrate, which is essential for the best possible mushroom growth.

Sustainability: Using algae as a substrate for mushroom cultivation can be more environmentally friendly than traditional substrates due to their high nutritional content, low water usage, and the potential to capture CO₂ during their growth. Algae are highly renewable and can be sourced sustainably, reducing the need to harvest natural resources for substrate production. They can also be used to reduce waste and promote sustainable resource management practices. By incorporating algae as a substrate for mushroom farming, we can contribute to more sustainable and eco-friendly agricultural practices while also potentially reducing the environmental impact of mushroom farming.

Growth-promoting compounds: The use of algae-based media as a substrate for mushroom cultivation offers the potential benefits of natural growth-promoting compounds such as hormones and growth regulators. These compounds can stimulate the growth of the mycelium and improve the density and strength of the fungal networks. By incorporating these natural growth-promoting compounds, the development and growth of the mycelium can be enhanced up to four folds, ultimately resulting in denser and more robust fungal networks. This represents an innovative approach to mushroom cultivation that has the potential to improve the yield and quality of mushroom production.

Additional Work: In various embodiments, the present invention provides a faster method of producing fungal mycelium when using microalgal and macroalgal biomass substrates. This study introduces a faster method for producing fungal mycelium by using micro and macro algae as substrates, either alone or combined with lignocellulosic biomass in various ratios. This method allows for the capture of CO₂ produced during mycelium production by utilizing micro or macro algae. This innovative closed-loop CO₂ capture system, reported here for the first time, aims to reduce carbon emissions during the mycelium production process. This new climate-smart cultivation technique was tested on the fungal species Hypsizygus ulmarius, Calocybe indica, Pleurotus ostreatus, Ganoderma lucidum, and Agaricus bisporus in culture plates. All five fungi demonstrated enhanced mycelium growth, reduced cultivation time, and increased biological efficiency compared to using lignocellulosic biomass or potato dextrose as substrates. This groundbreaking approach offers a sustainable solution to address the environmental impact of mycelium production while enhancing the industry's overall efficiency and productivity.

Introduction: Traditional mushroom cultivation methods that utilize lignocellulosic biomass—such as wood, straw, and agricultural waste—face several challenges. These substrates are composed of complex polymers, including cellulose, hemicellulose, and lignin, which form the structural components of plant cell walls. Cellulose is a linear polysaccharide made up of glucose units, while hemicellulose is a branched polysaccharide containing various sugar monomers. Lignin, on the other hand, is a complex aromatic polymer that provides strength and rigidity to plant tissues. The intricate structure of lignocellulosic biomass can impede nutrient accessibility for mushroom mycelium. Cellulose and hemicellulose are tightly bound to lignin, creating a resistant matrix that is difficult for the mycelium to penetrate and degrade.

This limited nutrient accessibility can result in slower mycelium growth rates and lower biological efficiency compared to grains. Additionally, breaking down lignocellulosic biomass requires a complex set of enzymes, including cellulases, hemicellulases, and lignin-degrading enzymes. While some fungal species, such as white-rot fungi, have the enzymatic capacity to degrade lignin, many cultivated species have limited lignin-degrading abilities. This limitation can further slow the colonization of the substrate by the mycelium, extending the overall cultivation time.

Moreover, producing mycelium significantly contributes to carbon dioxide (CO₂) emissions. As aerobic organisms, fungi require oxygen for their growth and metabolism, releasing CO₂ as a byproduct. For every pound of mycelium produced, a pound of CO₂ is released.

There is a growing interest in exploring alternative substrates and cultivation methods to address these challenges and promote sustainable mycelium production. Algae, including both macroalgae (seaweeds) and microalgae, have emerged as promising candidates for producing mycelium. Algae have a unique nutritional profile characterized by high protein content, essential amino acids, and various bioactive compounds. These nutrients can support the rapid growth and development of mushroom mycelium.

Moreover, algae can sequester CO₂ from the atmosphere through photosynthesis, converting it into biomass. By integrating algae cultivation with mushroom production, it is possible to create a closed-loop system where the CO₂ generated during mushroom cultivation is captured and utilized for algal growth. This approach can help mitigate mushroom production's carbon footprint and contribute to the development of a circular economy. The rapid growth rates of algae also make them an attractive substrate for mushroom cultivation. Microalgae have doubling times ranging from a few hours to a few days, depending on the species and cultivation conditions. This rapid biomass accumulation can provide a steady supply of substrate for mushroom production, reducing the reliance on seasonal or geographically limited lignocellulosic biomass sources.

This study presents a novel approach to mushroom and mycelium cultivation by utilizing algae-based substrates and a closed-loop CO₂ capture system. The primary objectives of this research are to:
Evaluate the effectiveness of algae-based substrates in supporting mycelium growth and fruiting body development of various mushroom species, including Hypsizygus ulmarius, Calocybe indica, Pleurotus ostreatus, and Ganoderma lucidum.
Compare the performance of algae-based substrates with traditional lignocellulosic substrates in terms of mycelium growth rate, biological efficiency, and cultivation time.
Develop a closed-loop system that integrates CO₂ capture from the mushroom cultivation process, conversion of CO₂ into sodium bicarbonate (NaHCO₃), and utilization of the converted CO₂ for algal biomass production.
Assess the potential of the proposed cultivation method to reduce CO₂ emissions and promote a circular economy by utilizing spent mushroom substrate as animal feed.

Without being bound by theory, we hypothesize that the use of algae-based substrates will enhance mycelium growth and biological efficiency and reduce cultivation time compared to traditional lignocellulosic substrates. Furthermore, integrating a closed-loop CO₂ capture system is expected to significantly reduce the carbon footprint associated with producing mycelium/mushroom while promoting a more sustainable and efficient cultivation process. The successful implementation of this innovative approach to mushroom cultivation has the potential to revolutionize the industry, offering a sustainable and eco-friendly alternative to traditional methods. By harnessing the potential of algae-based substrates and integrating a closed-loop CO₂ capture system, this study aims to develop a more sustainable, efficient, and environmentally conscious mushroom production process.

Method and material: This study was conducted in two phases to investigate the potential of using algae-based substrates for mushroom cultivation. In the first phase, mycelium growth on algae-based substrates was evaluated in Petri dishes under controlled laboratory conditions. The second phase focused on fruiting body production using the best-performing algae-based substrates from phase one, which were tested in mushroom cultivation bottles. Both phases used four mushroom species: Hypsizygus ulmarius, Calocybe indica, Pleurotus ostreatus, and Ganoderma lucidum.

Petridis mycelium production method: Macroalgae (dulse, and nori) and microalgae (Chlorella and Spirulina) powders were mixed with lignocellulosic biomass (wheat straw, wheat bran and corn flour) at 10% ratio dry basis. The mixtures were filled into 90 mm Petri dishes (20 g per dish) and sterilized by autoclaving at 121° C. for 30 minutes. The sterilized Petri dishes were inoculated with a 1 cm² piece of actively growing mycelium from pure cultures of the four mushroom species and incubated at 25° C. in the dark. On the last day of the experiment, mycelium was harvested and put into the oven at 80° C., over overnight, and dry weight was weighed.

To study the effect of salt on mycelium growth in a petri dish, macroalgae, and microalgae powder were mixed with water, centrifuged, and the supernatant was separated. The algae were then used for cultivation in petri dishes. However, because the concentration of algae was only 10%, the results for mycelium growth with and without salt removal did not show a significant difference.

Producing mushroom: Based on the Petridis experiments, the best-performing algae-based substrates for each fungal species were selected for producing fruiting body. The lignocellulosic biomass consisted of wheat straw (85%), wheat bran (10%), corn flour (5%), gypsum (1%), and lime (0.2%). Two substrate formulations were prepared: (1) 100% lignocellulosic biomass and (2) 50% lignocellulosic biomass+50% algae. The moisture content was adjusted to 70%, and the substrates were filled into polypropylene bottles (500 g per bottle), sealed with a plastic cap and a cotton plug, and sterilized by autoclaving at 121° C. for 30 minutes. The sterilized bottles were inoculated with a 5% (w/w) grain spawn of the respective mushroom species and incubated at 20° C. in the dark until complete mycelium colonization was achieved. The bottles were then transferred to a fruiting room maintained at 18-20° C., 85-90% relative humidity, and 500-1000 lux light intensity for 12 hours daily. Plastic caps were removed, and cotton plugs were loosened to initiate fruiting body formation. Fruiting bodies were harvested at maturity, and each bottle's fresh weight was recorded. All experiments were conducted in triplicate, and data were expressed as mean±standard deviation.

Pretreatment of Macroalgae: Using macroalgae as a substrate for mushroom cultivation presented two main challenges: high salt content and large particle size. In the first set of experiments, the mycelium could not grow on the actual substrate due to the high salt concentration. The macroalgae were pretreated before being incorporated into the substrate formulations to address these issues.

Salt removal: Macroalgae, being marine organisms, naturally contain high levels of salt, which can inhibit the growth and development of fungi. The macroalgae were soaked in fresh water to reduce the salt content for 2 hours. During this soaking period, the salt from the macroalgae diffused into the water, effectively lowering the salt concentration in the algal biomass. After soaking, a spinner drained the macroalgae and gently spun to remove excess water. This simple yet effective pretreatment method ensured that the salt content in the macroalgae was reduced to a level suitable for mushroom cultivation.

Size reduction: The large particle size of the macroalgae posed another challenge for their use as a substrate. Large particles can hinder the colonization of the substrate by the mycelium, leading to slower growth rates and reduced substrate utilization. To overcome this issue, the pretreated macroalgae were chopped into smaller pieces using a mechanical chopper. The target particle size was approximately 1-2 cm, which is like the size of the lignocellulosic biomass used in the study. The size reduction of the macroalgae increased their surface area, making them more accessible for the fungus and facilitating a more uniform distribution of the algal biomass within the substrate.

In some embodiments, these pretreatment methods addressed the macroalgae's high salt content and large particle size, rendering them more suitable for use as a substrate in mushroom cultivation. In some embodiments, the salt removal and size reduction processes were important steps in optimizing the performance of the algae-based substrates and ensuring the successful growth and development of the studied mushroom species.

The results of this study demonstrated that the use of algae-based substrates, either alone or in combination with lignocellulosic biomass, significantly influenced the mycelium growth and fruiting body production of the five studied mushroom species: Hypsizygus ulmarius, Calocybe indica, Pleurotus ostreatus, Ganoderma lucidum and Agaricus bisporus (button mushroom). Additionally, the cultivation of ELM mushrooms on algae-based substrates while producing mushrooms showed remarkable improvements in biological efficiency. The following sections will present the detailed results for each fungal species grown on different substrates, including lignocellulosic biomass, algae alone, and their combinations.

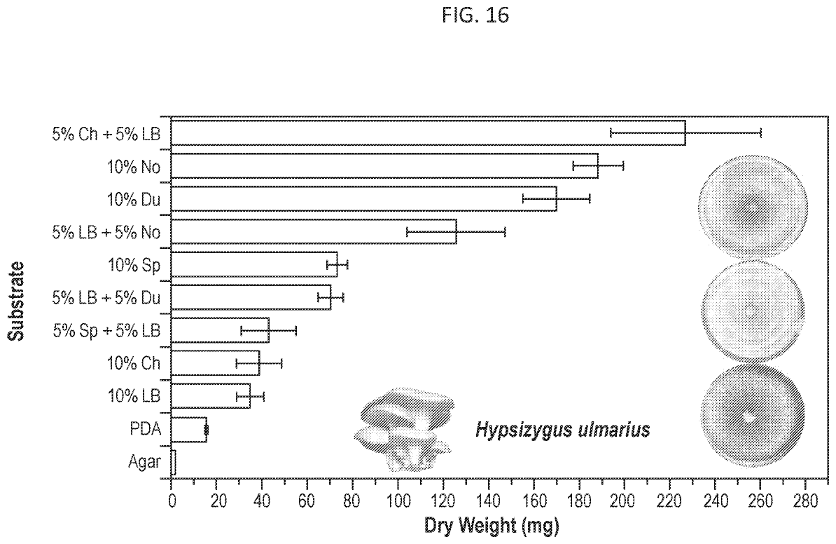

Hypsizygus ulmarius (ELM oyster mushroom): The results obtained for the ELM oyster mushroom (Hypsizygus ulmarius) reveal that using macroalgae as a substrate leads to a more than 2-fold increase in mycelium growth compared to the lignocellulosic biomass (FIG. 16). This finding highlights the superior performance of macroalgae as an alternative substrate for cultivating Hypsizygus ulmarius mycelium, demonstrating its potential to significantly enhance the efficiency and productivity of mushroom cultivation processes. In addition to macroalgae, the study also investigated using microalgae as substrates for Hypsizygus ulmarius mycelium growth.

The results showed that microalgae could also enhance mycelium growth compared to lignocellulosic biomass, although the improvement was not as substantial as that observed with macroalgae substrates. This finding suggests that both macroalgae and microalgae have the potential to serve as effective substrates for Hypsizygus ulmarius cultivation, with macroalgae showing superior performance. Combining chlorella with lignocellulosic biomass showed the highest mycelium growth.

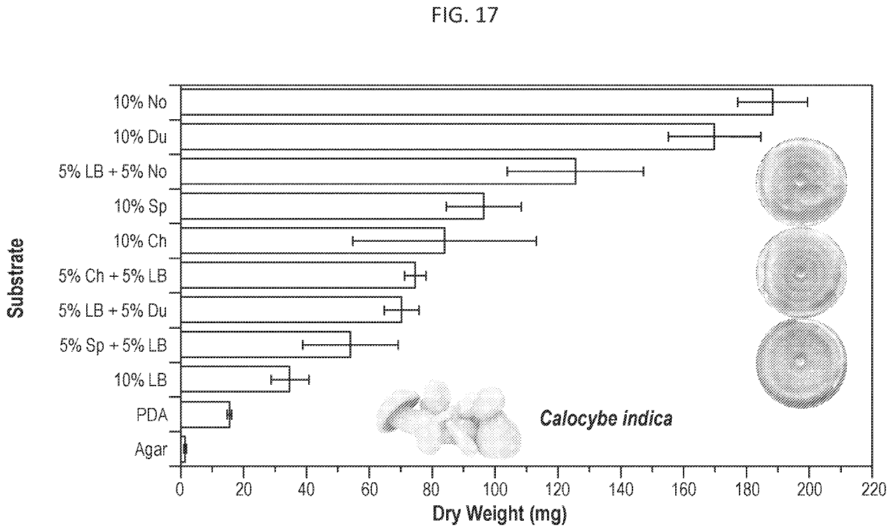

Calocybe indica (Milky white mushrooms): The Calocybe indica results demonstrate that using macroalgae, specifically Nori and Dulse, as substrates can significantly enhance mycelium growth compared to lignocellulosic biomass (FIG. 17). The experiments showed a remarkable 5.4-fold increase in mycelium growth when using Nori and a 4.9-fold increase when using Dulse as substrates, compared to the growth observed on lignocellulosic biomass alone. Furthermore, even when Nori and Dulse were mixed with lignocellulosic biomass, the mycelium growth of Calocybe indica was notably improved compared using lignocellulosic biomass alone.

The study also investigated using microalgae as substrates for Calocybe indica mycelium growth. While the results showed that microalgae could indeed enhance mycelium growth compared to lignocellulosic biomass, the improvement was not as substantial as that observed with macroalgae substrates. This difference in performance may be attributed to the distinct nutritional profiles and cell wall compositions of macroalgae and microalgae that influence the Calocybe indica mycelium growth.

Pleurotus ostreatus (Oyster Mushroom): The study found that microalgae substrates, such as Chlorella and Spirulina, performed significantly better than lignocellulosic biomass in supporting the mycelium growth of Pleurotus ostreatus. The superior performance of microalgae may be due to their high protein content, balanced amino acid composition, and the presence of various growth-promoting compounds, such as vitamins and minerals. The small size and relatively simple cell wall structure of microalgae may also contribute to improved mycelium growth by facilitating easier nutrient uptake and digestion.

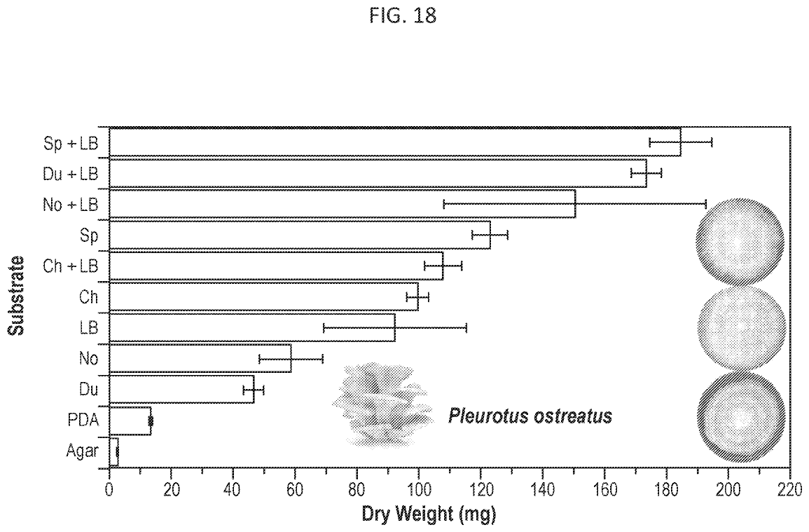

However, contrary to the previous statement, the macroalgae substrates, specifically Dulse and Nori, did not show a significant improvement in mycelium growth compared to lignocellulosic biomass for Pleurotus ostreatus. This suggests that not all macroalgae species may be equally effective in enhancing mycelium growth for this mushroom species (FIG. 18).

Interestingly, when microalgae were combined with lignocellulosic biomass, the mycelium growth of Pleurotus ostreatus was significantly enhanced, showing up to a 2-fold increase compared to the growth of lignocellulosic biomass alone. This synergistic effect suggests that microalgae can complement and improve the nutritional profile of lignocellulosic substrates, providing additional nutrients and growth-promoting factors that support the robust development of mycelium.

Furthermore, combining macroalgae and microalgae with lignocellulosic biomass also improved mycelium growth compared to lignocellulosic biomass alone. Although the macroalgae substrates alone may not have significantly improved, their incorporation alongside microalgae and lignocellulosic biomass positively impacts mycelium growth.

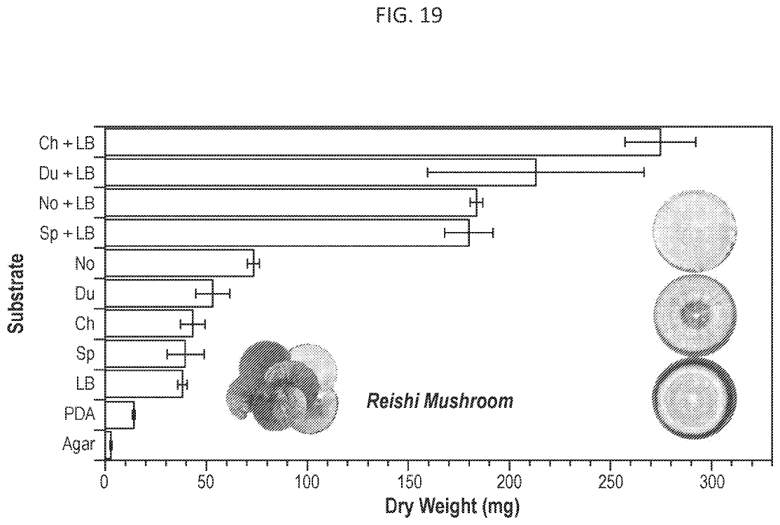

Ganoderma lucidum (Reishi Mushroom): The results obtained for Ganoderma lucidum showed new insights into the effects of using algae as substrates for mycelium growth. The study found that when macroalgae and microalgae were used alone as substrates, they did not significantly enhance the mycelium growth of Ganoderma lucidum compared to lignocellulosic biomass (FIG. 19).

However, a remarkable synergistic effect was observed when macroalgae and microalgae were combined with lignocellulosic biomass. The mycelium growth of Ganoderma lucidum was significantly enhanced, showing up to a 5-fold increase compared to the development of lignocellulosic biomass alone. This finding suggests that incorporating algae into traditional lignocellulosic substrates can dramatically improve the overall performance of the substrate for mycelium cultivation of Reishi mushrooms.

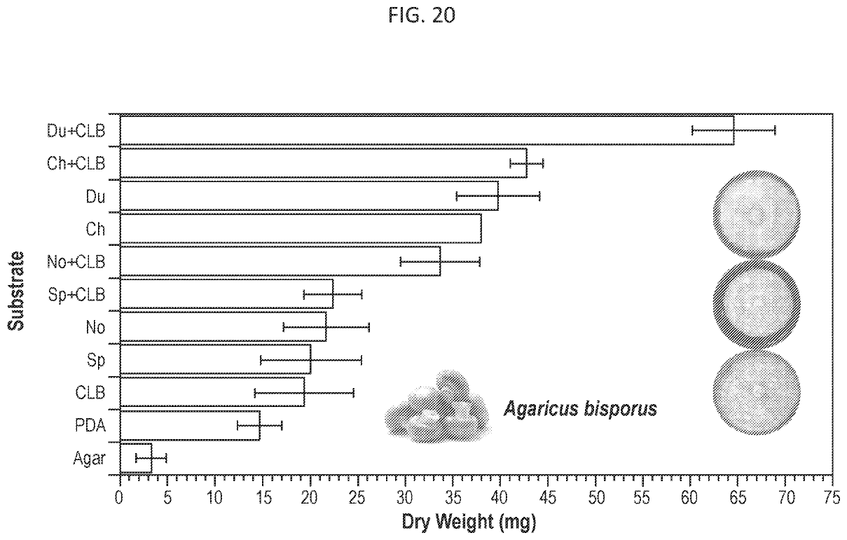

Agaricus bisporus (button mushrooms): The results for the button mushroom (Agaricus bisporus) cultivation in phase one highlighted the effectiveness of using Chlorella and Dulse as substrates, either alone or in combination with composted lignocellulosic biomass. When Chlorella and Dulse were used as the sole substrate components, they significantly enhanced the growth and biological efficiency of Agaricus bisporus compared to the control substrate, which consisted of 100% lignocellulosic biomass. The mushrooms grown on substrates containing only Chlorella or Dulse exhibited a remarkable 2-fold increase in growth and biological efficiency (FIG. 20).

The most impressive result, however, was observed when composted lignocellulosic biomass was combined with Dulse in a 50:50 ratio. This substrate formulation led to an outstanding 3-fold increase in the biological efficiency of Agaricus bisporus compared to the control. The synergistic effect between the composted lignocellulosic biomass and Dulse can be attributed to several factors.

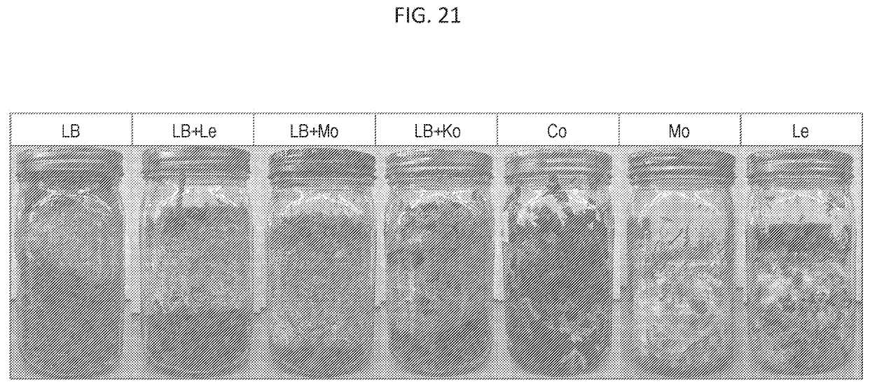

In another experiment, inoculation on actual substrates was conducted. ELM oyster mushroom (Hypsizygus ulmarius) was cultivated on lignocellulosic biomass and algae in this experiment. The first set of experiments involved using different types of macroalgae, such as sea moss, dulse, nori, kombu kelp, and sea lettuce. The results showed that certain kinds of macroalgae, like dulse and nori, were unsuitable for this experiment because they are soft and do not allow for proper air regulation. However, other types, such as sea moss and sea lettuce, proved to be suitable substrates. The experiments were conducted using pure macroalgae substrates or combined with lignocellulosic biomass. The results (FIG. 21) demonstrated that pure macroalgae substrates yielded the best outcomes.

After just 10 days, the pure macroalgae substrates were fully colonized, and the mushroom fruiting bodies started to appear, achieving a biological efficiency of 100%. In contrast, combining macroalgae and lignocellulosic biomass took two weeks to fully colonize. Lignocellulosic biomass alone required a significantly longer time, taking 4 to 6 weeks for complete colonization and initiating fruiting body formation. These findings highlight the potential of using pure macroalgae substrates, particularly sea moss and sea lettuce, to efficiently cultivate elm oyster mushrooms. The rapid colonization and high biological efficiency observed with pure macroalgae substrates demonstrate their superiority compared to the combination of macroalgae and lignocellulosic biomass or lignocellulosic biomass alone.

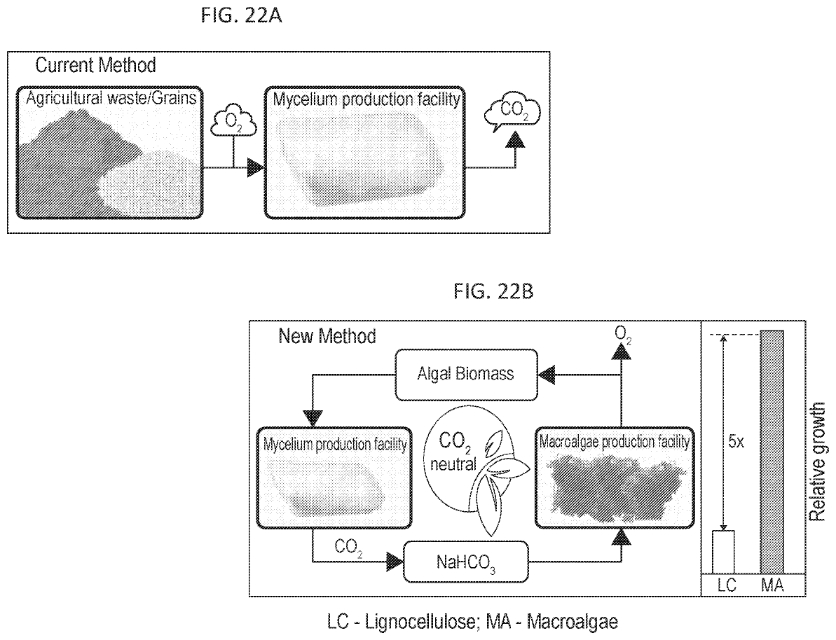

The technology described here presents a closed-loop system that integrates mushroom/mycelium cultivation, CO₂ capture, and algal biomass production, creating a sustainable and eco-friendly approach to mushroom production (FIG. 22B). The system operates as follows:

1. CO₂ capture from mushroom/mycelium chambers: The CO₂ generated during the mushroom/mycelium cultivation process is captured from the growth chambers. This step prevents the direct release of CO₂ into the atmosphere, reducing the cultivation process's carbon footprint.

2. Conversion of CO₂ to NaHCO₃: The captured CO₂ is then converted into sodium bicarbonate (NaHCO₃) through a chemical process. This conversion efficiently utilizes the captured CO₂ and provides a carbon source for subsequent production.

3. Transfer of NaHCO₃ to algal biomass system: The produced NaHCO₃ is transferred to the algal biomass production system. Sodium bicarbonate serves as a carbon source for the growth and development of algae, promoting efficient algal biomass production.

4. Algal biomass production: The algal biomass system utilizes the NaHCO₃ derived from the captured CO₂ to cultivate algae. Algae have a high growth rate and can efficiently convert CO₂ and carbon sources into biomass, effectively sequestering carbon and producing a valuable substrate for mushroom/mycelium cultivation.

5. Utilization of algal biomass as a substrate for mushroom/mycelium production: The produced algal biomass is then used to cultivate mushrooms and mycelium. As demonstrated in the previous experiments, algal biomass has shown promising results as a substrate, enhancing mycelium growth, reducing cultivation time, and improving biological efficiency compared to traditional lignocellulosic biomass substrates.

6. Utilization of spent mushroom substrate as animal feed: After the mushroom/mycelium cultivation process, the spent substrate, which is rich in nutrients, is utilized as animal feed. This step further maximizes the system's resource efficiency by providing a valuable by-product for animal nutrition.

The closed-loop system described here offers several advantages:
Sustainability: The system reduces mushroom production's carbon footprint by capturing and utilizing CO₂ from the mushroom/mycelium cultivation process. The conversion of CO₂ to NaHCO₃ and its subsequent use in algal biomass production creates a sustainable cycle that mitigates greenhouse gas emissions.
Resource efficiency: Integrating mushroom/mycelium cultivation, CO₂ capture, and algal biomass production optimizes resource utilization. The system efficiently recycles CO₂, carbon, and nutrients, minimizing waste and maximizing the productivity of each component.
Enhanced mushroom production: The use of algal biomass as a substrate for mushroom/mycelium cultivation has shown promising results in terms of improved growth, reduced cultivation time, and increased biological efficiency. This enhancement in mushroom production efficiency can lead to higher yields and reduced operational costs.
Valorization of by-products: Using spent mushroom substrate as animal feed adds value to the by-products of the mushroom cultivation process. This approach promotes a circular economy, where waste is minimized, and resources are maximized.

The closed-loop system presented here showcases a holistic approach to sustainable mushroom production, integrating CO₂ capture, algal biomass production, and the utilization of by-products. This technology can potentially revolutionize the mushroom industry, promoting eco-friendly practices, reducing environmental impact, and enhancing the efficiency and profitability of mushroom cultivation.

The use of pure macroalgae substrates not only reduces the cultivation time but also eliminates the need for lignocellulosic biomass, which is associated with longer colonization periods. This approach can lead to more efficient and sustainable mushroom production, as it minimizes the time and resources required for substrate preparation and fruiting body formation.

In conclusion, the use of macroalgae as a substrate for the cultivation of various mushroom species, including elm oyster mushroom (Hypsizygus ulmarius), milky white mushroom (Calocybe indica), oyster mushroom (Pleurotus ostreatus), and reishi mushroom (Ganoderma lucidum), has demonstrated remarkable potential in enhancing the efficiency and sustainability of mushroom production. The experiments conducted with pure macroalgae substrates and their combinations with lignocellulosic biomass have shown promising results across all the studied mushroom species.

Reduced cultivation time: Pure macroalgae substrates and their combinations with lignocellulosic biomass have shown faster colonization and fruiting body formation than lignocellulosic biomass alone. This reduction in cultivation time was observed across all the studied mushroom species, with some variations depending on the specific macroalgae used.

Enhanced biological efficiency: Using macroalgae substrates has resulted in higher biological efficiencies, indicating improved substrate utilization and increased mushroom yield. This enhancement was particularly evident in the case of Calocybe indica, where a 5.4-fold and 4.9-fold increase in mycelium growth was observed on Nori and Dulse substrates, respectively.

Energy and cost savings: The shorter cultivation times associated with macroalgae substrates translate to reduced energy consumption and operational costs. This benefit applies to all the studied mushroom species, as less time and resources are required for substrate preparation and maintenance.

Reduced CO₂ emissions: By incorporating macroalgae into the substrate, either alone or in combination with lignocellulosic biomass, the CO₂ emissions associated with mushroom production can be mitigated. Macroalgae could absorb CO₂ during their growth, offsetting the emissions generated during cultivation.

The findings of this study highlight the immense potential of macroalgae as a sustainable and efficient substrate for cultivating various fungal species to produce mycelium and mushrooms. Adopting this approach on a larger scale could revolutionize the mushroom and mycelium industry, leading to more environmentally friendly and cost-effective production methods across different edible fugal species. Further research should focus on optimizing the cultivation conditions for each fungal species, exploring the potential of other macroalgae species, and investigating the nutritional and functional properties of mushrooms grown on these substrates.

### Various Non-Limiting Embodiments of the Invention

In various embodiments, the present invention provides a material for producing, growing, and/or cultivating a fungus as described herein.

In various embodiments, the present invention provides a method for producing, growing, and/or cultivating a fungus as described herein.

In various embodiments, the present invention provides a material for producing, growing, and/or cultivating a mushroom as described herein.

In various embodiments, the present invention provides a method for producing, growing, and/or cultivating a mushroom as described herein.

In various embodiments, the present invention provides a material for producing, growing, and/or cultivating mycelium as described herein.

In various embodiments, the present invention provides a method for producing, growing, and/or cultivating mycelium as described herein.

In some embodiments, the material is algae.

In some embodiments, the algae is microalgae, macroalgae, or combination thereof.

In some embodiments, the material is at least one microalgae.

In some embodiments, the material is at least one macroalgae.

In some embodiments, the material is at least one microalgae, at least one macroalgae, or combination thereof.

In some embodiments, the material is a combination of lignocellulosic biomass and algae.

In some embodiments, the algae is microalgae, macroalgae, or combination thereof.

In some embodiments, the material comprises algae.

In some embodiments, the algae is microalgae, macroalgae, or combination thereof.

In some embodiments, the method comprises using a substrate to produce, grow, and/or cultivate the fungus, wherein the substrate is algae.

In some embodiments, the algae is microalgae, macroalgae, or combination thereof.

In some embodiments, the method comprises using a substrate to produce, grow, and/or cultivate the fungus, wherein the substrate is at least one microalgae.

In some embodiments, the method comprises using a substrate to produce, grow, and/or cultivate the fungus, wherein the substrate is at least one macroalgae.

In some embodiments, the method comprises using a substrate to produce, grow, and/or cultivate the fungus, wherein the substrate is at least one microalgae, at least one macroalgae, or combination thereof.

In some embodiments, the method comprises using a substrate to produce, grow, and/or cultivate the fungus, wherein the substrate is a combination of lignocellulosic biomass and algae.

In some embodiments, the algae is microalgae, macroalgae, or combination thereof.

In some embodiments, the method comprises using a substrate to produce, grow, and/or cultivate the fungus, wherein the substrate comprises algae.

In some embodiments, the substrate further comprises lignocellulosic biomass.

In some embodiments, the algae is microalgae, macroalgae, or combination thereof.

In some embodiments, the method comprises using a substrate to produce, grow, and/or cultivate the mushroom, wherein the substrate is algae.

In some embodiments, the algae is microalgae, macroalgae, or combination thereof.

In some embodiments, the method comprises using a substrate to produce, grow, and/or cultivate the mushroom, wherein the substrate is at least one microalgae.

In some embodiments, the method comprises using a substrate to produce, grow, and/or cultivate the mushroom, wherein the substrate is at least one macroalgae.

In some embodiments, the method comprises using a substrate to produce, grow, and/or cultivate the mushroom, wherein the substrate is at least one microalgae, at least one macroalgae, or combination thereof.

In some embodiments, the method comprises using a substrate to produce, grow, and/or cultivate the mushroom, wherein the substrate is a combination of lignocellulosic biomass and algae.

In some embodiments, the algae is microalgae, macroalgae, or combination thereof.

In some embodiments, the method comprises using a substrate to produce, grow, and/or cultivate the mushroom, wherein the substrate comprises algae.

In some embodiments, the substrate further comprises lignocellulosic biomass.

In some embodiments, the algae is microalgae, macroalgae, or combination thereof.

In some embodiments, the method comprises using a substrate to produce, grow, and/or cultivate the mycelium, wherein the substrate is algae.

In some embodiments, the algae is microalgae, macroalgae, or combination thereof.

In some embodiments, the method comprises using a substrate to produce, grow, and/or cultivate the mycelium, wherein the substrate is at least one microalgae.

In some embodiments, the method comprises using a substrate to produce, grow, and/or cultivate the mycelium, wherein the substrate is at least one macroalgae.

In some embodiments, the method comprises using a substrate to produce, grow, and/or cultivate the mycelium, wherein the substrate is at least one microalgae, at least one macroalgae, or combination thereof.

In some embodiments, wherein the method comprises using a substrate to produce, grow, and/or cultivate the mycelium, wherein the substrate is a combination of lignocellulosic biomass and algae.

In some embodiments, the algae is microalgae, macroalgae, or combination thereof.

In some embodiments, the method comprises using a substrate to produce, grow, and/or cultivate the mycelium, wherein the substrate comprises algae.

In some embodiments, the substrate further comprises lignocellulosic biomass.

In some embodiments, the algae is microalgae, macroalgae, or combination thereof.

Additional embodiments include:

Embodiment 1. A material for producing, growing, and/or cultivating a fungus as described herein.

Embodiment 2. A method for producing, growing, and/or cultivating a fungus as described herein.

Embodiment 3. A material for producing, growing, and/or cultivating a mushroom as described herein.

Embodiment 4. A method for producing, growing, and/or cultivating a mushroom as described herein.

Embodiment 5. A material for producing, growing, and/or cultivating mycelium as described herein.

Embodiment 6. A method for producing, growing, and/or cultivating mycelium as described herein.

Embodiment 7. The material of any one of embodiments 1, 3, or 5, wherein the material is algae.

Embodiment 8. The material of embodiment 7, wherein the algae is microalgae, macroalgae, or combination thereof.

Embodiment 9. The material of any one of embodiments 1, 3, or 5, wherein the material is at least one microalgae.

Embodiment 10. The material of any one of embodiments 1, 3, or 5, wherein the material is at least one macroalgae.

Embodiment 11. The material of any one of embodiments 1, 3, or 5, wherein the material is at least one microalgae, at least one macroalgae, or combination thereof.

Embodiment 12. The material of any one of embodiments 1, 3, or 5, wherein the material is a combination of lignocellulosic biomass and algae.

Embodiment 13. The material of embodiment 12, wherein the algae is microalgae, macroalgae, or combination thereof.

Embodiment 14. The material of any one of embodiments 1, 3, or 5, wherein the material comprises algae.

Embodiment 15. The material of embodiment 14, wherein the algae is microalgae, macroalgae, or combination thereof.

Embodiment 16. The method of embodiment 2, wherein the method comprises using a substrate to produce, grow, and/or cultivate the fungus, wherein the substrate is algae.

Embodiment 17. The method of embodiment 16, wherein the algae is microalgae, macroalgae, or combination thereof.

Embodiment 18. The method of embodiment 2, wherein the method comprises using a substrate to produce, grow, and/or cultivate the fungus, wherein the substrate is at least one microalgae.

Embodiment 19. The method of embodiment 2, wherein the method comprises using a substrate to produce, grow, and/or cultivate the fungus, wherein the substrate is at least one macroalgae.

Embodiment 20. The method of embodiment 2, wherein the method comprises using a substrate to produce, grow, and/or cultivate the fungus, wherein the substrate is at least one microalgae, at least one macroalgae, or combination thereof.

Embodiment 21. The method of embodiment 2, wherein the method comprises using a substrate to produce, grow, and/or cultivate the fungus, wherein the substrate is a combination of lignocellulosic biomass and algae.

Embodiment 22. The method of embodiment 21, wherein the algae is microalgae, macroalgae, or combination thereof.

Embodiment 23. The method of embodiment 2, wherein the method comprises using a substrate to produce, grow, and/or cultivate the fungus, wherein the substrate comprises algae.

Embodiment 24. The method of embodiment 23, wherein the substrate further comprises lignocellulosic biomass.

Embodiment 25. The method of embodiment 23 or embodiment 24, wherein the algae is microalgae, macroalgae, or combination thereof.

Embodiment 26. The method of embodiment 4, wherein the method comprises using a substrate to produce, grow, and/or cultivate the mushroom, wherein the substrate is algae.

Embodiment 27. The method of embodiment 26, wherein the algae is microalgae, macroalgae, or combination thereof,

Embodiment 28. The method of embodiment 4, wherein the method comprises using a substrate to produce, grow, and/or cultivate the mushroom, wherein the substrate is at least one microalgae.

Embodiment 29. The method of embodiment 4, wherein the method comprises using a substrate to produce, grow, and/or cultivate the mushroom, wherein the substrate is at least one macroalgae.

Embodiment 30. The method of embodiment 4, wherein the method comprises using a substrate to produce, grow, and/or cultivate the mushroom, wherein the substrate is at least one microalgae, at least one macroalgae, or combination thereof.

Embodiment 31. The method of embodiment 4, wherein the method comprises using a substrate to produce, grow, and/or cultivate the mushroom, wherein the substrate is a combination of lignocellulosic biomass and algae.

Embodiment 32. The method of embodiment 31, wherein the algae is microalgae, macroalgae, or combination thereof.

Embodiment 33. The method of embodiment 4, wherein the method comprises using a substrate to produce, grow, and/or cultivate the mushroom, wherein the substrate comprises algae.

Embodiment 34. The method of embodiment 33, wherein the substrate further comprises lignocellulosic biomass.

Embodiment 35. The method of embodiment 33 or embodiment 34, wherein the algae is microalgae, macroalgae, or combination thereof.

Embodiment 36. The method of embodiment 6, wherein the method comprises using a substrate to produce, grow, and/or cultivate the mycelium, wherein the substrate is algae.

Embodiment 37. The method of embodiment 36, wherein the algae is microalgae, macroalgae, or combination thereof.

Embodiment 38. The method of embodiment 6, wherein the method comprises using a substrate to produce, grow, and/or cultivate the mycelium, wherein the substrate is at least one microalgae.

Embodiment 39. The method of embodiment 6, wherein the method comprises using a substrate to produce, grow, and/or cultivate the mycelium, wherein the substrate is at least one macroalgae.

Embodiment 40. The method of embodiment 6, wherein the method comprises using a substrate to produce, grow, and/or cultivate the mycelium, wherein the substrate is at least one microalgae, at least one macroalgae, or combination thereof.

Embodiment 41. The method of embodiment 6, wherein the method comprises using a substrate to produce, grow, and/or cultivate the mycelium, wherein the substrate is a combination of lignocellulosic biomass and algae.

Embodiment 42. The method of embodiment 41, wherein the algae is microalgae, macroalgae, or combination thereof.

Embodiment 43. The method of embodiment 6, wherein the method comprises using a substrate to produce, grow, and/or cultivate the mycelium, wherein the substrate comprises algae.

Embodiment 44. The method of embodiment 43, wherein the substrate further comprises lignocellulosic biomass.

Embodiment 45. The method of embodiment 43 or embodiment 44, wherein the algae is microalgae, macroalgae, or combination thereof.

Additional embodiments include the following:

In various embodiments the present invention provides a substrate for cultivating at least one fungus, the substrate comprising at least one algae. In various embodiments, the present invention provides a substrate for cultivating at least one fungus, the substrate consisting of at least one algae. In various embodiments, the present invention provides a substrate for cultivating at least one fungus, the substrate consisting essentially of at least one algae. In some embodiments, the at least one fungus is not Cordyceps militaris.

In various embodiments the present invention provides a substrate for cultivating at least one mushroom, the substrate comprising at least one algae. In various embodiments, the present invention provides a substrate for cultivating at least one mushroom, the substrate consisting of at least one algae. In various embodiments, the present invention provides a substrate for cultivating at least one mushroom, the substrate consisting essentially of at least one algae. In some embodiments, the at least one mushroom is not Cordyceps militaris.

In various embodiments the present invention provides a substrate for cultivating at least one mycelium, the substrate comprising at least one algae. In various embodiments, the present invention provides a substrate for cultivating at least one mycelium, the substrate consisting of at least one algae. In various embodiments, the present invention provides a substrate for cultivating at least one mycelium, the substrate consisting essentially of at least one algae. In some embodiments, the at least one mycelium is not Cordyceps militaris. In some embodiments, the at least one mycelium is at least one fungal mycelium. In some embodiments, the at least one mycelium is at least one mushroom mycelium. In some embodiments, the at least one mycelium is at least one fungal mycelium. In some embodiments, the at least one mycelium is at least one fungus mycelium. In some embodiments, the at least one mycelium is from fungus. In some embodiments, the at least one mycelium is from mushroom. In some embodiments, the at least one mycelium is from yeast. In some embodiments, the at least one mycelium is not from yeast. In some embodiments, the at least one mycelium is from mold. In some embodiments, the at least one mycelium is not from mold. In some embodiments, the at least one mycelium is not from mold and is not from yeast.

In some embodiments, the spores are fungal spores. In some embodiments, the spores are mushroom spores. In some embodiments, the spores are fungus spores. In some embodiments, the spores are yeast spores. In some embodiments, the spores are not yeast spores. In some embodiments, the spores are mold spores. In some embodiments, the spores are not mold spores. In some embodiments, the spores are not mold spores and are not yeast spores.

In various embodiments, the present invention provides a fungus cultivation substrate, the substrate comprising at least one algae. In various embodiments, the present invention provides a fungus cultivation substrate, the substrate consisting of at least one algae. In various embodiments, the present invention provides a fungus cultivation substrate, the substrate consisting essentially of at least one algae.

In various embodiments, the present invention provides a mushroom cultivation substrate, the substrate comprising at least one algae. In various embodiments, the present invention provides a mushroom cultivation substrate, the substrate consisting of at least one algae. In various embodiments, the present invention provides a mushroom cultivation substrate, the substrate consisting essentially of at least one algae.

In various embodiments, the present invention provides a mycelium cultivation substrate, the substrate comprising at least one algae. In various embodiments, the present invention provides a mycelium cultivation substrate, the substrate consisting of at least one algae. In various embodiments, the present invention provides a mycelium cultivation substrate, the substrate consisting essentially of at least one algae. In some embodiments, the mycelium is a fungal mycelium. In some embodiments, the mycelium is a mushroom mycelium. In some embodiments, the mycelium is at least one fungus mycelium. In some embodiments, the mycelium is from fungus. In some embodiments, the mycelium is from mushroom. In some embodiments, the mycelium is from yeast. In some embodiments, the mycelium is not from yeast. In some embodiments, the mycelium is from mold. In some embodiments, the mycelium is not from mold. In some embodiments, the mycelium is not from mold and is not from yeast.

In various embodiments, the present invention provides a substrate for cultivating at least one fungus, the substrate comprising at least one algae, or at least one cyanobacteria, or a combination thereof. In various embodiments, the present invention provides a substrate for cultivating at least one fungus, the substrate consisting of at least one algae, or at least one cyanobacteria, or a combination thereof. In various embodiments, the present invention provides a substrate for cultivating at least one fungus, the substrate consisting essentially of at least one algae, or at least one cyanobacteria, or a combination thereof.

In various embodiments, the present invention provides a substrate for cultivating at least one mushroom, the substrate comprising at least one algae, or at least one cyanobacteria, or a combination thereof. In various embodiments, the present invention provides a substrate for cultivating at least one mushroom, the substrate consisting of at least one algae, or at least one cyanobacteria, or a combination thereof. In various embodiments, the present invention provides a substrate for cultivating at least one mushroom, the substrate consisting essentially of at least one algae, or at least one cyanobacteria, or a combination thereof.

In various embodiments, the present invention provides a substrate for cultivating at least one mycelium, the substrate comprising at least one algae, or at least one cyanobacteria, or a combination thereof. In various embodiments, the present invention provides a substrate for cultivating at least one mycelium, the substrate consisting of at least one algae, or at least one cyanobacteria, or a combination thereof. In various embodiments, the present invention provides a substrate for cultivating at least one mycelium, the substrate consisting essentially of at least one algae, or at least one cyanobacteria, or a combination thereof. In some embodiments, the at least one mycelium is at least one fungal mycelium. In some embodiments, the at least one mycelium is at least one mushroom mycelium. In some embodiments, the at least one mycelium is at least one fungus mycelium. In some embodiments, the at least one mycelium is from fungus. In some embodiments, the at least one mycelium is from mushroom. In some embodiments, the at least one mycelium is from yeast. In some embodiments, the at least one mycelium is not from yeast. In some embodiments, the at least one mycelium is from mold. In some embodiments, the at least one mycelium is not from mold. In some embodiments, the at least one mycelium is not from mold and is not from yeast.

In various embodiments the present invention provides, a method for cultivating at least one fungus, the method comprising: providing a substrate of the present invention; inoculating the substrate with fungus spores or fugus mycelium; and applying conditions to stimulate growth of at least one fungus.

In various embodiments the present invention provides, a method for cultivating at least one mushroom, the method comprising: providing a substrate of the present invention; inoculating the substrate with mushroom spores or mushroom mycelium; and applying conditions to stimulate growth of at least one mushroom.

In various embodiments the present invention provides, a method for cultivating at least one mycelium, the method comprising: providing a substrate of the present invention; inoculating the substrate with mycelium spores; and applying conditions to stimulate growth of at least one mycelium. In some embodiments, the at least one mycelium is at least one fungal mycelium. In some embodiments, the at least one mycelium is at least one mushroom mycelium. In some embodiments, the mycelium spores are fungal mycelium spores. In some embodiments, the mycelium spores are mushroom mycelium spores.

In various embodiments the present invention provides, a method for cultivating at least one fungus and for producing algal biomass, the method comprising: providing a substrate of the present invention; inoculating the substrate with fungus spores or fungus mycelium; applying conditions to stimulate growth of at least one fungus; capturing carbon dioxide produced during the cultivation of the at least one fungus; converting the captured carbon dioxide into sodium bicarbonate; and utilizing the sodium bicarbonate as a carbon source to produce algal biomass.

In various embodiments the present invention provides, a method for cultivating at least one mushroom and for producing algal biomass, the method comprising: providing a substrate of the present invention; inoculating the substrate with mushroom spores or mushroom mycelium; applying conditions to stimulate growth of at least one mushroom; capturing carbon dioxide produced during the cultivation of the at least one mushroom; converting the captured carbon dioxide into sodium bicarbonate; and utilizing the sodium bicarbonate as a carbon source to produce algal biomass.

In various embodiments the present invention provides, a method for cultivating at least one mycelium and for producing algal biomass, the method comprising: providing a substrate of the present invention; inoculating the substrate with mycelium spores; applying conditions to stimulate growth of at least one mycelium; capturing carbon dioxide produced during the cultivation of the at least one mycelium; converting the captured carbon dioxide into sodium bicarbonate; and utilizing the sodium bicarbonate as a carbon source to produce algal biomass. In some embodiments, the at least one mycelium is at least one fungal mycelium. In some embodiments, the at least one mycelium is at least one mushroom mycelium. In some embodiments, the mycelium spores are fungal mycelium spores. In some embodiments, the mycelium spores are mushroom mycelium spores.

In various embodiments the present invention provides, a system for cultivating at least one mushroom and for producing algal biomass, the system comprising: a first chamber, wherein the first chamber is adapted for cultivating at least one mushroom and capturing carbon dioxide produced during cultivation of the at least one mushroom, and wherein the first chamber comprises a substrate of the present invention; a second chamber, wherein the second chamber is in communication with the first chamber, and wherein the second chamber is adapted to convert the captured carbon dioxide into sodium bicarbonate; and a third chamber, wherein the third chamber is in communication with the second chamber, and wherein the third chamber is adapted for using the sodium bicarbonate as a carbon source for producing algal biomass.

In some embodiments, the system for cultivating the at least one mushroom and for producing algal biomass further comprises, a fourth chamber, wherein the fourth chamber is adapted for storing algal biomass. In some embodiments, the fourth chamber is in communication with the third chamber. In some embodiments, the fourth chamber is in communication with the first chamber. In some embodiments, the fourth chamber is in communication with the third chamber and the first chamber. In some embodiments, the first chamber is in communication with the fourth chamber. In some embodiments, the third chamber is in communication with the first chamber. In some embodiments, the first chamber is in communication with the third chamber. In some embodiments, the algal biomass stored in the fourth chamber is produced in the third chamber. In some embodiments, the algal biomass stored in the fourth chamber is used to prepare a substrate of the present invention.

In some embodiments, the third chamber comprises an outlet for releasing and/or transferring and/or transporting oxygen (O₂). In some embodiments, the oxygen (O₂) is released to the atmosphere. In some embodiments, the oxygen (O₂) is transferred and/or transported to the first chamber. In some embodiments, the oxygen (O₂) is produced in the third chamber during the production of the algal biomass. In some embodiments, the oxygen (O₂) is produced in the third chamber. In some embodiments, the third chamber is adapted for producing the oxygen (O₂). In some embodiments, the third chamber is adapted for transferring and/or transporting the oxygen (O₂). In some embodiments, the third chamber is adapted for producing and/or transferring and/or transporting the oxygen (O₂).

In some embodiments, the first chamber contains or comprises a substrate of the present invention. In some embodiments, the first chamber contains or comprises at least one mushroom. In some embodiments, the at least one mushroom is in communication with the substrate of the present invention. In some embodiments, the first chamber contains or comprises oxygen. In some embodiments, the first chamber contains or comprises carbon dioxide. In some embodiments, the second chamber contains or comprises carbon dioxide. In some embodiments, the second chamber contains or comprises sodium bicarbonate. In some embodiments, the third chamber contains or comprises algal biomass. In some embodiments, the third chamber contains or comprises sodium bicarbonate. In some embodiments, the third chamber contains or comprises oxygen. In some embodiments, the fourth chamber contains or comprises algal biomass.

In various embodiments the present invention provides, a system for cultivating at least one fungus and for producing algal biomass, the system comprising: a first chamber, wherein the first chamber is adapted for cultivating at least one fungus and capturing carbon dioxide produced during cultivation of the at least one fungus, and wherein the first chamber comprises a substrate of the present invention; a second chamber, wherein the second chamber is in communication with the first chamber, and wherein the second chamber is adapted to convert the captured carbon dioxide into sodium bicarbonate; and a third chamber, wherein the third chamber is in communication with the second chamber, and wherein the third chamber is adapted for using the sodium bicarbonate as a carbon source for producing algal biomass.

In some embodiments, the system for cultivating the at least one fungus and for producing algal biomass further comprises, a fourth chamber, wherein the fourth chamber is adapted for storing algal biomass. In some embodiments, the fourth chamber is in communication with the third chamber. In some embodiments, the fourth chamber is in communication with the first chamber. In some embodiments, the fourth chamber is in communication with the third chamber and the first chamber. In some embodiments, the first chamber is in communication with the fourth chamber. In some embodiments, the third chamber is in communication with the first chamber. In some embodiments, the first chamber is in communication with the third chamber. In some embodiments, the algal biomass stored in the fourth chamber is produced in the third chamber. In some embodiments, the algal biomass stored in the fourth chamber is used to prepare a substrate of the present invention.

In some embodiments, the third chamber comprises an outlet for releasing and/or transferring and/or transporting oxygen (O₂). In some embodiments, the oxygen (O₂) is released to the atmosphere. In some embodiments, the oxygen (O₂) is transferred and/or transported to the first chamber. In some embodiments, the oxygen (O₂) is produced in the third chamber during the production of the algal biomass. In some embodiments, the oxygen (O₂) is produced in the third chamber. In some embodiments, the third chamber is adapted for producing the oxygen (O₂). In some embodiments, the third chamber is adapted for transferring and/or transporting the oxygen (O₂). In some embodiments, the third chamber is adapted for producing and/or transferring and/or transporting the oxygen (O₂).

In some embodiments, the first chamber contains or comprises a substrate of the present invention. In some embodiments, the first chamber contains or comprises at least one fungus. In some embodiments, the at least one fungus is in communication with the substrate of the present invention. In some embodiments, the first chamber contains or comprises oxygen. In some embodiments, the first chamber contains or comprises carbon dioxide. In some embodiments, the second chamber contains or comprises carbon dioxide. In some embodiments, the second chamber contains or comprises sodium bicarbonate. In some embodiments, the third chamber contains or comprises algal biomass. In some embodiments, the third chamber contains or comprises oxygen. In some embodiments, the third chamber contains or comprises sodium bicarbonate. In some embodiments, the fourth chamber contains or comprises algal biomass.

In various embodiments the present invention provides, a system for cultivating at least one mycelium and for producing algal biomass, the system comprising: a first chamber, wherein the first chamber is adapted for cultivating at least one mycelium and capturing carbon dioxide produced during cultivation of the at least one mycelium, and wherein the first chamber comprises a substrate of the present invention; a second chamber, wherein the second chamber is in communication with the first chamber, and wherein the second chamber is adapted to convert the captured carbon dioxide into sodium bicarbonate; and a third chamber, wherein the third chamber is in communication with the second chamber, and wherein the third chamber is adapted for using the sodium bicarbonate as a carbon source for producing algal biomass. In some embodiments, the at least one mycelium is at least one fungal mycelium. In some embodiments, the at least one mycelium is at least one mushroom mycelium. In some embodiments, the at least one mycelium is at least one fungus mycelium. In some embodiments, the at least one mycelium is from fungus. In some embodiments, the at least one mycelium is from mushroom. In some embodiments, the at least one mycelium is from yeast. In some embodiments, the at least one mycelium is not from yeast. In some embodiments, the at least one mycelium is from mold. In some embodiments, the at least one mycelium is not from mold. In some embodiments, the at least one mycelium is not from mold and is not from yeast.

In some embodiments, the system for cultivating the at least one mycelium and for producing algal biomass further comprises, a fourth chamber, wherein the fourth chamber is adapted for storing algal biomass. In some embodiments, the fourth chamber is in communication with the third chamber. In some embodiments, the fourth chamber is in communication with the first chamber. In some embodiments, the fourth chamber is in communication with the third chamber and the first chamber. In some embodiments, the first chamber is in communication with the fourth chamber. In some embodiments, the third chamber is in communication with the first chamber. In some embodiments, the first chamber is in communication with the third chamber. In some embodiments, the algal biomass stored in the fourth chamber is produced in the third chamber. In some embodiments, the algal biomass stored in the fourth chamber is used to prepare a substrate of the present invention.

In some embodiments, the third chamber comprises an outlet for releasing and/or transferring and/or transporting oxygen (O₂). In some embodiments, the oxygen (O₂) is released to the atmosphere. In some embodiments, the oxygen (O₂) is transferred and/or transported to the first chamber. In some embodiments, the oxygen (O₂) is produced in the third chamber during the production of the algal biomass. In some embodiments, the oxygen (O₂) is produced in the third chamber. In some embodiments, the third chamber is adapted for producing the oxygen (O₂). In some embodiments, the third chamber is adapted for transferring and/or transporting the oxygen (O₂). In some embodiments, the third chamber is adapted for producing and/or transferring and/or transporting the oxygen (O₂).

In some embodiments, the first chamber contains or comprises a substrate of the present invention. In some embodiments, the first chamber contains or comprises at least one mycelium. In some embodiments, the at least one mycelium is in communication with the substrate of the present invention. In some embodiments, the first chamber contains or comprises oxygen. In some embodiments, the first chamber contains or comprises carbon dioxide. In some embodiments, the second chamber contains or comprises carbon dioxide. In some embodiments, the second chamber contains or comprises sodium bicarbonate. In some embodiments, the third chamber contains or comprises algal biomass. In some embodiments, the third chamber contains or comprises oxygen. In some embodiments, the third chamber contains or comprises sodium bicarbonate. In some embodiments, the fourth chamber contains or comprises algal biomass.

In various embodiments the present invention provides, a system for cultivating at least one mushroom and for producing algal biomass, the system comprising: a first vessel, wherein the first vessel is adapted for cultivating at least one mushroom and capturing carbon dioxide produced during cultivation of the at least one mushroom, and wherein the first vessel comprises a substrate of the present invention; a second vessel, wherein the second vessel is in communication with the first vessel, and wherein the second vessel is adapted to convert the captured carbon dioxide into sodium bicarbonate; and a third vessel, wherein the third vessel is in communication with the second vessel, and wherein the third vessel is adapted for using the sodium bicarbonate as a carbon source for producing algal biomass.

In some embodiments, the system for cultivating the at least one mushroom and for producing algal biomass further comprises, a fourth vessel, wherein the fourth vessel is adapted for storing algal biomass. In some embodiments, the fourth vessel is in communication with the third vessel. In some embodiments, the fourth vessel is in communication with the first vessel. In some embodiments, the fourth vessel is in communication with the third vessel and the first vessel. In some embodiments, the first vessel is in communication with the fourth vessel. In some embodiments, the third vessel is in communication with the first vessel. In some embodiments, the first vessel is in communication with the third vessel. In some embodiments, the algal biomass stored in the fourth vessel is produced in the third vessel. In some embodiments, the algal biomass stored in the fourth vessel is used to prepare a substrate of the present invention.

In some embodiments, the third vessel comprises an outlet for releasing and/or transferring and/or transporting oxygen (O₂). In some embodiments, the oxygen (O₂) is released to the atmosphere. In some embodiments, the oxygen (O₂) is transferred and/or transported to the first vessel. In some embodiments, the oxygen (O₂) is produced in the third vessel during the production of the algal biomass. In some embodiments, the oxygen (O₂) is produced in the third vessel. In some embodiments, the third vessel is adapted for producing the oxygen (O₂). In some embodiments, the third vessel is adapted for transferring and/or transporting the oxygen (O₂). In some embodiments, the third vessel is adapted for producing and/or transferring and/or transporting the oxygen (O₂).

In some embodiments, the first vessel contains or comprises a substrate of the present invention. In some embodiments, the first vessel contains or comprises at least one mushroom. In some embodiments, the at least one mushroom is in communication with the substrate of the present invention. In some embodiments, the first vessel contains or comprises oxygen. In some embodiments, the first vessel contains or comprises carbon dioxide. In some embodiments, the second vessel contains or comprises carbon dioxide. In some embodiments, the second vessel contains or comprises sodium bicarbonate. In some embodiments, the third vessel contains or comprises algal biomass. In some embodiments, the third vessel contains or comprises oxygen. In some embodiments, the third vessel contains or comprises sodium bicarbonate. In some embodiments, the fourth vessel contains or comprises algal biomass.

In various embodiments the present invention provides, a system for cultivating at least one fungus and for producing algal biomass, the system comprising: a first vessel, wherein the first vessel is adapted for cultivating at least one fungus and capturing carbon dioxide produced during cultivation of the at least one fungus, and wherein the first vessel comprises a substrate of the present invention; a second vessel, wherein the second vessel is in communication with the first vessel, and wherein the second vessel is adapted to convert the captured carbon dioxide into sodium bicarbonate; and a third vessel, wherein the third vessel is in communication with the second vessel, and wherein the third vessel is adapted for using the sodium bicarbonate as a carbon source for producing algal biomass.

In some embodiments, the system for cultivating the at least one fungus and for producing algal biomass further comprises, a fourth vessel, wherein the fourth vessel is adapted for storing algal biomass. In some embodiments, the fourth vessel is in communication with the third vessel. In some embodiments, the fourth vessel is in communication with the first vessel. In some embodiments, the fourth vessel is in communication with the third vessel and the first vessel. In some embodiments, the first vessel is in communication with the fourth vessel. In some embodiments, the third vessel is in communication with the first vessel. In some embodiments, the first vessel is in communication with the third vessel. In some embodiments, the algal biomass stored in the fourth vessel is produced in the third vessel. In some embodiments, the algal biomass stored in the fourth vessel is used to prepare a substrate of the present invention.

In some embodiments, the third vessel comprises an outlet for releasing and/or transferring and/or transporting oxygen (O₂). In some embodiments, the oxygen (O₂) is released to the atmosphere. In some embodiments, the oxygen (O₂) is transferred and/or transported to the first vessel. In some embodiments, the oxygen (O₂) is produced in the third vessel during the production of the algal biomass. In some embodiments, the oxygen (O₂) is produced in the third vessel. In some embodiments, the third vessel is adapted for producing the oxygen (O₂). In some embodiments, the third vessel is adapted for transferring and/or transporting the oxygen (O₂). In some embodiments, the third vessel is adapted for producing and/or transferring and/or transporting the oxygen (O₂).

In some embodiments, the first vessel contains or comprises a substrate of the present invention. In some embodiments, the first vessel contains or comprises at least one fungus. In some embodiments, the at least one fungus is in communication with the substrate of the present invention. In some embodiments, the first vessel contains or comprises oxygen. In some embodiments, the first vessel contains or comprises carbon dioxide. In some embodiments, the second vessel contains or comprises carbon dioxide. In some embodiments, the second vessel contains or comprises sodium bicarbonate. In some embodiments, the third vessel contains or comprises algal biomass. In some embodiments, the third vessel contains or comprises oxygen. In some embodiments, the third vessel contains or comprises sodium bicarbonate. In some embodiments, the fourth vessel contains or comprises algal biomass.

In various embodiments the present invention provides, a system for cultivating at least one mycelium and for producing algal biomass, the system comprising: a first vessel, wherein the first vessel is adapted for cultivating at least one mycelium and capturing carbon dioxide produced during cultivation of the at least one mycelium, and wherein the first vessel comprises a substrate of the present invention; a second vessel, wherein the second vessel is in communication with the first vessel, and wherein the second vessel is adapted to convert the captured carbon dioxide into sodium bicarbonate; and a third vessel, wherein the third vessel is in communication with the second vessel, and wherein the third vessel is adapted for using the sodium bicarbonate as a carbon source for producing algal biomass. In some embodiments, the at least one mycelium is at least one fungal mycelium. In some embodiments, the at least one mycelium is at least one mushroom mycelium. In some embodiments, the at least one mycelium is at least one fungus mycelium. In some embodiments, the at least one mycelium is from fungus. In some embodiments, the at least one mycelium is from mushroom. In some embodiments, the at least one mycelium is from yeast. In some embodiments, the at least one mycelium is not from yeast. In some embodiments, the at least one mycelium is from mold. In some embodiments, the at least one mycelium is not from mold. In some embodiments, the at least one mycelium is not from mold and is not from yeast.

In some embodiments, the system for cultivating the at least one mycelium and for producing algal biomass further comprises, a fourth vessel, wherein the fourth vessel is adapted for storing algal biomass. In some embodiments, the fourth vessel is in communication with the third vessel. In some embodiments, the fourth vessel is in communication with the first vessel. In some embodiments, the fourth vessel is in communication with the third vessel and the first vessel. In some embodiments, the first vessel is in communication with the fourth vessel. In some embodiments, the third vessel is in communication with the first vessel. In some embodiments, the first vessel is in communication with the third vessel. In some embodiments, the algal biomass stored in the fourth vessel is produced in the third vessel. In some embodiments, the algal biomass stored in the fourth vessel is used to prepare a substrate of the present invention.

In some embodiments, the third vessel comprises an outlet for releasing and/or transferring and/or transporting oxygen (O₂). In some embodiments, the oxygen (O₂) is released to the atmosphere. In some embodiments, the oxygen (O₂) is transferred and/or transported to the first vessel. In some embodiments, the oxygen (O₂) is produced in the third vessel during the production of the algal biomass. In some embodiments, the oxygen (O₂) is produced in the third vessel. In some embodiments, the third vessel is adapted for producing the oxygen (O₂). In some embodiments, the third vessel is adapted for transferring and/or transporting the oxygen (O₂). In some embodiments, the third vessel is adapted for producing and/or transferring and/or transporting the oxygen (O₂).

In some embodiments, the first vessel contains or comprises a substrate of the present invention. In some embodiments, the first vessel contains or comprises at least one mycelium. In some embodiments, the at least one mycelium is in communication with the substrate of the present invention. In some embodiments, the first vessel contains or comprises oxygen. In some embodiments, the first vessel contains or comprises carbon dioxide. In some embodiments, the second vessel contains or comprises carbon dioxide. In some embodiments, the second vessel contains or comprises sodium bicarbonate. In some embodiments, the third vessel contains or comprises algal biomass. In some embodiments, the third vessel contains or comprises oxygen. In some embodiments, the third vessel contains or comprises sodium bicarbonate. In some embodiments, the fourth vessel contains or comprises algal biomass.

In various embodiments the present invention provides, a system for cultivating at least one mushroom and for producing algal biomass, the system comprising: a first facility, wherein the first facility is adapted for cultivating at least one mushroom and capturing carbon dioxide produced during cultivation of the at least one mushroom, and wherein the first facility comprises a substrate of the present invention; a second facility, wherein the second facility is in communication with the first facility, and wherein the second facility is adapted to convert the captured carbon dioxide into sodium bicarbonate; and a third facility, wherein the third facility is in communication with the second facility, and wherein the third facility is adapted for using the sodium bicarbonate as a carbon source for producing algal biomass.

In some embodiments, the system for cultivating the at least one mushroom and for producing algal biomass further comprises, a fourth facility, wherein the fourth facility is adapted for storing algal biomass. In some embodiments, the fourth facility is in communication with the third facility. In some embodiments, the fourth facility is in communication with the first facility. In some embodiments, the fourth facility is in communication with the third facility and the first facility. In some embodiments, the first facility is in communication with the fourth facility. In some embodiments, the third facility is in communication with the first facility. In some embodiments, the first facility is in communication with the third facility. In some embodiments, the algal biomass stored in the fourth facility is produced in the third facility. In some embodiments, the algal biomass stored in the fourth facility is used to prepare a substrate of the present invention.

In some embodiments, the third facility comprises an outlet for releasing and/or transferring and/or transporting oxygen (O₂). In some embodiments, the oxygen (O₂) is released to the atmosphere. In some embodiments, the oxygen (O₂) is transferred and/or transported to the first facility. In some embodiments, the oxygen (O₂) is produced in the third facility during the production of the algal biomass. In some embodiments, the oxygen (O₂) is produced in the third facility. In some embodiments, the third facility is adapted for producing the oxygen (O₂). In some embodiments, the third facility is adapted for transferring and/or transporting the oxygen (O₂). In some embodiments, the third facility is adapted for producing and/or transferring and/or transporting the oxygen (O₂).

In some embodiments, the first facility contains or comprises a substrate of the present invention. In some embodiments, the first facility contains or comprises at least one mushroom. In some embodiments, the at least one mushroom is in communication with the substrate of the present invention. In some embodiments, the first facility contains or comprises oxygen. In some embodiments, the first facility contains or comprises carbon dioxide. In some embodiments, the second facility contains or comprises carbon dioxide. In some embodiments, the second facility contains or comprises sodium bicarbonate. In some embodiments, the third facility contains or comprises algal biomass. In some embodiments, the third facility contains or comprises oxygen. In some embodiments, the third facility contains or comprises sodium bicarbonate. In some embodiments, the fourth facility contains or comprises algal biomass.

In various embodiments the present invention provides, a system for cultivating at least one fungus and for producing algal biomass, the system comprising: a first facility, wherein the first facility is adapted for cultivating at least one fungus and capturing carbon dioxide produced during cultivation of the at least one fungus, and wherein the first facility comprises a substrate of the present invention; a second facility, wherein the second facility is in communication with the first facility, and wherein the second facility is adapted to convert the captured carbon dioxide into sodium bicarbonate; and a third facility, wherein the third facility is in communication with the second facility, and wherein the third facility is adapted for using the sodium bicarbonate as a carbon source for producing algal biomass.

In some embodiments, the system for cultivating the at least one fungus and for producing algal biomass further comprises, a fourth facility, wherein the fourth facility is adapted for storing algal biomass. In some embodiments, the fourth facility is in communication with the third facility. In some embodiments, the fourth facility is in communication with the first facility. In some embodiments, the fourth facility is in communication with the third facility and the first facility. In some embodiments, the first facility is in communication with the fourth facility. In some embodiments, the third facility is in communication with the first facility. In some embodiments, the first facility is in communication with the third facility. In some embodiments, the algal biomass stored in the fourth facility is produced in the third facility. In some embodiments, the algal biomass stored in the fourth facility is used to prepare a substrate of the present invention.

In some embodiments, the third facility comprises an outlet for releasing and/or transferring and/or transporting oxygen (O₂). In some embodiments, the oxygen (O₂) is released to the atmosphere. In some embodiments, the oxygen (O₂) is transferred and/or transported to the first facility. In some embodiments, the oxygen (O₂) is produced in the third facility during the production of the algal biomass. In some embodiments, the oxygen (O₂) is produced in the third facility. In some embodiments, the third facility is adapted for producing the oxygen (O₂). In some embodiments, the third facility is adapted for transferring and/or transporting the oxygen (O₂). In some embodiments, the third facility is adapted for producing and/or transferring and/or transporting the oxygen (O₂).

In some embodiments, the first facility contains or comprises a substrate of the present invention. In some embodiments, the first facility contains or comprises at least one fungus. In some embodiments, the at least one fungus is in communication with the substrate of the present invention. In some embodiments, the first facility contains or comprises oxygen. In some embodiments, the first facility contains or comprises carbon dioxide. In some embodiments, the second facility contains or comprises carbon dioxide. In some embodiments, the second facility contains or comprises sodium bicarbonate. In some embodiments, the third facility contains or comprises algal biomass. In some embodiments, the third facility contains or comprises sodium bicarbonate. In some embodiments, the third facility contains or comprises oxygen. In some embodiments, the fourth facility contains or comprises algal biomass.

In various embodiments the present invention provides, a system for cultivating at least one mycelium and for producing algal biomass, the system comprising: a first facility, wherein the first facility is adapted for cultivating at least one mycelium and capturing carbon dioxide produced during cultivation of the at least one mycelium, and wherein the first facility comprises a substrate of the present invention; a second facility, wherein the second facility is in communication with the first facility, and wherein the second facility is adapted to convert the captured carbon dioxide into sodium bicarbonate; and a third facility, wherein the third facility is in communication with the second facility, and wherein the third facility is adapted for using the sodium bicarbonate as a carbon source for producing algal biomass. In some embodiments, the at least one mycelium is at least one fungal mycelium. In some embodiments, the at least one mycelium is at least one mushroom mycelium. In some embodiments, the at least one mycelium is at least one fungus mycelium. In some embodiments, the at least one mycelium is from fungus. In some embodiments, the at least one mycelium is from mushroom. In some embodiments, the at least one mycelium is from yeast. In some embodiments, the at least one mycelium is not from yeast. In some embodiments, the at least one mycelium is from mold. In some embodiments, the at least one mycelium is not from mold. In some embodiments, the at least one mycelium is not from mold and is not from yeast.

In some embodiments, the system for cultivating the at least one mycelium and for producing algal biomass further comprises, a fourth facility, wherein the fourth facility is adapted for storing algal biomass. In some embodiments, the fourth facility is in communication with the third facility. In some embodiments, the fourth facility is in communication with the first facility. In some embodiments, the fourth facility is in communication with the third facility and the first facility. In some embodiments, the first facility is in communication with the fourth facility. In some embodiments, the third facility is in communication with the first facility. In some embodiments, the first facility is in communication with the third facility. In some embodiments, the algal biomass stored in the fourth facility is produced in the third facility. In some embodiments, the algal biomass stored in the fourth facility is used to prepare a substrate of the present invention.

In some embodiments, the third facility comprises an outlet for releasing and/or transferring and/or transporting oxygen (O₂). In some embodiments, the oxygen (O₂) is released to the atmosphere. In some embodiments, the oxygen (O₂) is transferred and/or transported to the first facility. In some embodiments, the oxygen (O₂) is produced in the third facility during the production of the algal biomass. In some embodiments, the oxygen (O₂) is produced in the third facility. In some embodiments, the third facility is adapted for producing the oxygen (O₂). In some embodiments, the third facility is adapted for transferring and/or transporting the oxygen (O₂). In some embodiments, the third facility is adapted for producing and/or transferring and/or transporting the oxygen (O₂).

In some embodiments, the first facility contains or comprises a substrate of the present invention. In some embodiments, the first facility contains or comprises at least one mycelium. In some embodiments, the at least one mycelium is in communication with the substrate of the present invention. In some embodiments, the first facility contains or comprises oxygen. In some embodiments, the first facility contains or comprises carbon dioxide. In some embodiments, the second facility contains or comprises carbon dioxide. In some embodiments, the second facility contains or comprises sodium bicarbonate. In some embodiments, the third facility contains or comprises algal biomass. In some embodiments, the third facility contains or comprises sodium bicarbonate. In some embodiments, the third facility contains or comprises oxygen. In some embodiments, the fourth facility contains or comprises algal biomass.

In various embodiments, the present invention provides a substrate for cultivating at least one fungal mycelium or at least one mushroom, the substrate comprising: at least one algae. In various embodiments, the present invention provides a substrate for cultivating at least one fungal mycelium or at least one mushroom, the substrate comprising: at least one algae, provided that the at least one algae is not Spirulina maxima or Chlorella vulgaris.

In various embodiments, the present invention provides a substrate for cultivating at least one fungal mycelium, the substrate comprising: at least one algae. In various embodiments, the present invention provides a substrate for cultivating at least one fungal mycelium, the substrate comprising: at least one algae, provided that the at least one algae is not Spirulina maxima or Chlorella vulgaris.

In various embodiments, the present invention provides a substrate for cultivating at least one mushroom, the substrate comprising: at least one algae. In various embodiments, the present invention provides a substrate for cultivating at least one mushroom, the substrate comprising: at least one algae, provided that the at least one algae is not Spirulina maxima or Chlorella vulgaris.

In various embodiments, the present invention provides a substrate for cultivating at least one mushroom mycelium, the substrate comprising: at least one algae. In various embodiments, the present invention provides a substrate for cultivating at least one mushroom mycelium, the substrate comprising: at least one algae, provided that the at least one algae is not Spirulina maxima or Chlorella vulgaris.

In various embodiments, the present invention provides a substrate for cultivating at least one mycelium, the substrate comprising: at least one algae. In various embodiments, the present invention provides a substrate for cultivating at least one mycelium, the substrate comprising: at least one algae, provided that the at least one algae is not Spirulina maxima or Chlorella vulgaris.

In some embodiments, the at least one algae is treated to remove at least a portion of salt from the at least one algae. In some embodiments, the at least one algae is treated to remove salt from the at least one algae. In some embodiments, the at least one algae is pretreated to remove at least a portion of salt from the at least one algae. In some embodiments, the at least one algae is pretreated to remove salt from the at least one algae. In some embodiments, the treatment is physical, chemical, mechanical, electrical, or biological, or any combination thereof. In some embodiments, the pretreatment is physical, chemical, mechanical, electrical, or biological, or any combination thereof. In some embodiments, the treatment to remove at least a portion of the salt from the at least one algae is performed before the at least one algae is used as the substrate. In some embodiments, the pretreatment to remove at least a portion of the salt from the at least one algae is performed before the at least one algae is used as the substrate. In some embodiments, the at least one algae is treated or pretreated with water to remove at least a portion of salt from the at least one algae. In some embodiments, the at least one algae is treated or pretreated with water to remove salt from the at least one algae.

In some embodiments, the at least one algae is not treated to remove at least a portion of salt from the at least one algae. In some embodiments, the at least one algae is not treated to remove salt from the at least one algae. In some embodiments, the at least one algae is not pretreated to remove at least a portion of salt from the at least one algae. In some embodiments, the at least one algae is not pretreated to remove salt from the at least one algae.

In some embodiments, the at least one algae is optionally treated to remove at least a portion of salt from the at least one algae. In some embodiments, the at least one algae is optionally treated to remove salt from the at least one algae. In some embodiments, the at least one algae is optionally pretreated to remove at least a portion of salt from the at least one algae. In some embodiments, the at least one algae is optionally pretreated to remove salt from the at least one algae.

In some embodiments, the at least one microalgae is treated to remove at least a portion of salt from the at least one microalgae. In some embodiments, the at least one microalgae is treated to remove salt from the at least one microalgae. In some embodiments, the at least one microalgae is pretreated to remove at least a portion of salt from the at least one microalgae. In some embodiments, the at least one microalgae is pretreated to remove salt from the at least one microalgae. In some embodiments, the treatment is physical, chemical, mechanical, electrical, or biological, or any combination thereof. In some embodiments, the pretreatment is physical, chemical, mechanical, electrical, or biological, or any combination thereof. In some embodiments, the treatment to remove at least a portion of the salt from the at least one microalgae is performed before the at least one microalgae is used as the substrate. In some embodiments, the pretreatment to remove at least a portion of the salt from the at least one microalgae is performed before the at least one microalgae is used as the substrate. In some embodiments, the at least one microalgae is treated or pretreated with water to remove at least a portion of salt from the at least one microalgae. In some embodiments, the at least one microalgae is treated or pretreated with water to remove salt from the at least one microalgae.

In some embodiments, the at least one microalgae is not treated to remove at least a portion of salt from the at least one microalgae. In some embodiments, the at least one microalgae is not treated to remove salt from the at least one microalgae. In some embodiments, the at least one microalgae is not pretreated to remove at least a portion of salt from the at least one microalgae. In some embodiments, the at least one microalgae is not pretreated to remove salt from the at least one microalgae.

In some embodiments, the at least one microalgae is optionally treated to remove at least a portion of salt from the at least one microalgae. In some embodiments, the at least one microalgae is optionally treated to remove salt from the at least one microalgae. In some embodiments, the at least one microalgae is optionally pretreated to remove at least a portion of salt from the at least one microalgae. In some embodiments, the at least one microalgae is optionally pretreated to remove salt from the at least one microalgae.

In some embodiments, the at least one macroalgae is treated to remove at least a portion of salt from the at least one macroalgae. In some embodiments, the at least one macroalgae is treated to remove salt from the at least one macroalgae. In some embodiments, the at least one macroalgae is pretreated to remove at least a portion of salt from the at least one macroalgae. In some embodiments, the at least one macroalgae is pretreated to remove salt from the at least one macroalgae. In some embodiments, the treatment is physical, chemical, mechanical, electrical, or biological, or any combination thereof. In some embodiments, the pretreatment is physical, chemical, mechanical, electrical, or biological, or any combination thereof. In some embodiments, the treatment to remove at least a portion of the salt from the at least one macroalgae is performed before the at least one macroalgae is used as the substrate. In some embodiments, the pretreatment to remove at least a portion of the salt from the at least one macroalgae is performed before the at least one macroalgae is used as the substrate. In some embodiments, the at least one macroalgae is treated or pretreated with water to remove at least a portion of salt from the at least one macroalgae. In some embodiments, the at least one macroalgae is treated or pretreated with water to remove salt from the at least one macroalgae.

In some embodiments, the at least one macroalgae is not treated to remove at least a portion of salt from the at least one macroalgae. In some embodiments, the at least one macroalgae is not treated to remove salt from the at least one macroalgae. In some embodiments, the at least one macroalgae is not pretreated to remove at least a portion of salt from the at least one macroalgae. In some embodiments, the at least one macroalgae is not pretreated to remove salt from the at least one macroalgae.

In some embodiments, the at least one macroalgae is optionally treated to remove at least a portion of salt from the at least one macroalgae. In some embodiments, the at least one macroalgae is optionally treated to remove salt from the at least one macroalgae. In some embodiments, the at least one macroalgae is optionally pretreated to remove at least a portion of salt from the at least one macroalgae. In some embodiments, the at least one macroalgae is optionally pretreated to remove salt from the at least one macroalgae.

In some embodiments, the at least one algae is processed to remove at least a portion of salt from the at least one algae. In some embodiments, the at least one algae is processed to remove salt from the at least one algae. In some embodiments, the at least one algae is preprocessed to remove at least a portion of salt from the at least one algae. In some embodiments, the at least one algae is preprocessed to remove salt from the at least one algae. In some embodiments, the process is physical, chemical, mechanical, electrical, or biological, or any combination thereof. In some embodiments, the preprocess is physical, chemical, mechanical, electrical, or biological, or any combination thereof. In some embodiments, the process to remove at least a portion of the salt from the at least one algae is performed before the at least one algae is used as the substrate. In some embodiments, the preprocess to remove at least a portion of the salt from the at least one algae is performed before the at least one algae is used as the substrate. In some embodiments, the at least one algae is processed or preprocessed with water to remove at least a portion of salt from the at least one algae. In some embodiments, the at least one algae is processed or preprocessed with water to remove salt from the at least one algae.

In some embodiments, the at least one algae is not processed to remove at least a portion of salt from the at least one algae. In some embodiments, the at least one algae is not processed to remove salt from the at least one algae. In some embodiments, the at least one algae is not preprocessed to remove at least a portion of salt from the at least one algae. In some embodiments, the at least one algae is not preprocessed to remove salt from the at least one algae.

In some embodiments, the at least one algae is optionally processed to remove at least a portion of salt from the at least one algae. In some embodiments, the at least one algae is optionally processed to remove salt from the at least one algae. In some embodiments, the at least one algae is optionally preprocessed to remove at least a portion of salt from the at least one algae. In some embodiments, the at least one algae is optionally preprocessed to remove salt from the at least one algae.

In some embodiments, the at least one microalgae is processed to remove at least a portion of salt from the at least one microalgae. In some embodiments, the at least one microalgae is processed to remove salt from the at least one microalgae. In some embodiments, the at least one microalgae is preprocessed to remove at least a portion of salt from the at least one microalgae. In some embodiments, the at least one microalgae is preprocessed to remove salt from the at least one microalgae. In some embodiments, the process is physical, chemical, mechanical, electrical, or biological, or any combination thereof. In some embodiments, the preprocess is physical, chemical, mechanical, electrical, or biological, or any combination thereof. In some embodiments, the process to remove at least a portion of the salt from the at least one microalgae is performed before the at least one microalgae is used as the substrate. In some embodiments, the preprocess to remove at least a portion of the salt from the at least one microalgae is performed before the at least one microalgae is used as the substrate. In some embodiments, the at least one microalgae is processed or preprocessed with water to remove at least a portion of salt from the at least one microalgae. In some embodiments, the at least one microalgae is processed or preprocessed with water to remove salt from the at least one microalgae.

In some embodiments, the at least one microalgae is not processed to remove at least a portion of salt from the at least one microalgae. In some embodiments, the at least one microalgae is not processed to remove salt from the at least one microalgae. In some embodiments, the at least one microalgae is not preprocessed to remove at least a portion of salt from the at least one microalgae. In some embodiments, the at least one microalgae is not preprocessed to remove salt from the at least one microalgae.

In some embodiments, the at least one microalgae is optionally processed to remove at least a portion of salt from the at least one microalgae. In some embodiments, the at least one microalgae is optionally processed to remove salt from the at least one microalgae. In some embodiments, the at least one microalgae is optionally preprocessed to remove at least a portion of salt from the at least one microalgae. In some embodiments, the at least one microalgae is optionally preprocessed to remove salt from the at least one microalgae

In some embodiments, the at least one macroalgae is processed to remove at least a portion of salt from the at least one macroalgae. In some embodiments, the at least one macroalgae is processed to remove salt from the at least one macroalgae. In some embodiments, the at least one macroalgae is preprocessed to remove at least a portion of salt from the at least one macroalgae. In some embodiments, the at least one macroalgae is preprocessed to remove salt from the at least one macroalgae. In some embodiments, the process is physical, chemical, mechanical, electrical, or biological, or any combination thereof. In some embodiments, the preprocess is physical, chemical, mechanical, electrical, or biological, or any combination thereof. In some embodiments, the process to remove at least a portion of the salt from the at least one macroalgae is performed before the at least one macroalgae is used as the substrate. In some embodiments, the preprocess to remove at least a portion of the salt from the at least one macroalgae is performed before the at least one macroalgae is used as the substrate. In some embodiments, the at least one macroalgae is processed or preprocessed with water to remove at least a portion of salt from the at least one macroalgae. In some embodiments, the at least one macroalgae is processed or preprocessed with water to remove salt from the at least one macroalgae.

In some embodiments, the at least one macroalgae is not processed to remove at least a portion of salt from the at least one macroalgae. In some embodiments, the at least one macroalgae is not processed to remove salt from the at least one macroalgae. In some embodiments, the at least one macroalgae is not preprocessed to remove at least a portion of salt from the at least one macroalgae. In some embodiments, the at least one macroalgae is not preprocessed to remove salt from the at least one macroalgae.

In some embodiments, the at least one macroalgae is optionally processed to remove at least a portion of salt from the at least one macroalgae. In some embodiments, the at least one macroalgae is optionally processed to remove salt from the at least one macroalgae. In some embodiments, the at least one macroalgae is optionally preprocessed to remove at least a portion of salt from the at least one macroalgae. In some embodiments, the at least one macroalgae is optionally preprocessed to remove salt from the at least one macroalgae.

In some embodiments, the size of microalgae can range from a few micrometers (um) to a few hundred micrometers (um). In some embodiments, the size of microalgae can range from a few micrometers (um) to several hundred micrometers (um). In some embodiments, the size of microalgae is 0.1 um to 50 um, or 1 um to 50 um, or 2 um to 50 um, or 0.1 um to 100 um, or 1 um to 100 um, or 2 um to 100 um, or 0.1 to 200 um, or 1 um to 200 um, or 2 um to 200 um, or 0.1 um to 300 um, or 1 um to 300 um, or 2 um to 300 um, or 0.1 um to 400 um, or 1 um to 400 um, or 2 um to 400 um, or 0.1 um to 500, or 1 um to 500 um, or 2 um to 500 um, or 0.1 um to 600 um, or 1 um to 600 um, or 2 um to 600 um, or 0.1 um to 700 um, or 1 um to 700 um, or 2 um to 700 um, or any size between these ranges.

In some embodiments, the at least one algae is treated to reduce the size of the at least one algae. In some embodiments, the at least one algae is treated to reduce the particle size of the at least one algae. In some embodiments, the at least one algae is pretreated to reduce the size of the at least one algae. In some embodiments, the at least one algae is pretreated to reduce the particle size of the at least one algae. In some embodiments, the size or particle size of the at least one algae is reduced relative to or compared to the native size or original size of the at least one algae before being treated or pretreated or processed or preprocessed to reduce the size or particle size of the at least one algae. In some embodiments, the size or particle size of the at least one algae is reduced relative to or compared to the size or particle size of the at least one algae before being treated or pretreated or processed or preprocessed to reduce the size or particle size of the at least one algae.

In some embodiments, the at least one algae is processed to reduce the size of the at least one algae. In some embodiments, the at least one algae is processed to reduce the particle size of the at least one algae. In some embodiments, the at least one algae is preprocessed to reduce the size of the at least one algae. In some embodiments, the at least one algae is preprocessed to reduce the particle size of the at least one algae.

In some embodiments, the reduced size or a reduced particle size of the at least one algae is 0.1 cm to 10 cm, 0.1 cm to 9 cm, 0.1 cm to 8 cm, 0.1 cm to 7 cm, 0.1 cm to 6 cm, 0.1 cm to 5 cm, 0.1 cm to 4 cm, 0.1 cm to 3 cm, 0.1 cm to 2 cm, or 0.1 cm to 1 cm, or any size between these ranges. In some embodiments, the reduced size or reduced particle size of the at least one algae is 1 cm to 10 cm, 1 cm to 9 cm, 1 cm to 8 cm, 1 cm to 7 cm, 1 cm to 6 cm, 1 cm to 5 cm, 1 cm to 4 cm, 1 cm to 3 cm, or 1 cm to 2 cm, or any size between these ranges. In some embodiments, the reduced size or reduced particle size of the at least one algae is less than 1 cm, less than 2 cm, less than 3 cm, less than 4 cm, less than 5 cm, less than 6 cm, less than 7 cm, less than 8 cm, less than 9 cm, or less than 10 cm. In some embodiments, the reduced size or reduced particle size of the at least one algae is 0.1 cm, 0.2 cm, 0.3 cm, 0.4 cm, 0.5 cm, 0.6 cm, 0.7 cm, 0.8 cm, 0.9 cm, 1.0 cm, 1.1 cm, 1.2 cm, 1.3 cm, 1.4 cm, 1.5 cm, 1.6 cm, 1.7 cm, 1.8 cm, 1.9 cm, 2.0 cm, 3.0 cm, 4.0 cm, 5.0 cm, 6.0 cm, 7.0 cm, 8.0 cm, 9.0 cm, or 10.0 cm. In some embodiments, the treatment, pretreatment, process, or preprocess is physical, chemical, mechanical, electrical, or biological, or any combination thereof.

In some embodiments, the at least one macroalgae is treated to reduce the size of the at least one macroalgae. In some embodiments, the at least one macroalgae is treated to reduce the particle size of the at least one macroalgae. In some embodiments, the at least one macroalgae is pretreated to reduce the size of the at least one macroalgae. In some embodiments, the at least one macroalgae is pretreated to reduce the particle size of the at least one macroalgae. In some embodiments, the size or particle size of the at least one macroalgae is reduced relative to or compared to the native size or original size of the at least one macroalgae before being treated or pretreated or processed or preprocessed to reduce the size or particle size of the at least one macroalgae. In some embodiments, the size or particle size of the at least one macroalgae is reduced relative to or compared to the size or particle size of the at least one macroalgae before being treated or pretreated or processed or preprocessed to reduce the size or particle size of the at least one macroalgae.

In some embodiments, the at least one macroalgae is processed to reduce the size of the at least one macroalgae. In some embodiments, the at least one macroalgae is processed to reduce the particle size of the at least one macroalgae. In some embodiments, the at least one macroalgae is preprocessed to reduce the size of the at least one macroalgae. In some embodiments, the at least one macroalgae is preprocessed to reduce the particle size of the at least one macroalgae.

In some embodiments, the reduced size of the at least one macroalgae is 0.1 cm to 10 cm, 0.1 cm to 9 cm, 0.1 cm to 8 cm, 0.1 cm to 7 cm, 0.1 cm to 6 cm, 0.1 cm to 5 cm, 0.1 cm to 4 cm, 0.1 cm to 3 cm, 0.1 cm to 2 cm, or 0.1 cm to 1 cm, or any size between these ranges. In some embodiments, the reduced size of the at least one macroalgae is 1 cm to 10 cm, 1 cm to 9 cm, 1 cm to 8 cm, 1 cm to 7 cm, 1 cm to 6 cm, 1 cm to 5 cm, 1 cm to 4 cm, 1 cm to 3 cm, or 1 cm to 2 cm, or any size between these ranges. In some embodiments, the reduced size of the at least one macroalgae is less than 1 cm, less than 2 cm, less than 3 cm, less than 4 cm, less than 5 cm, less than 6 cm, less than 7 cm, less than 8 cm, less than 9 cm, or less than 10 cm. In some embodiments, the reduced size of the at least one macroalgae is 0.1 cm, 0.2 cm, 0.3 cm, 0.4 cm, 0.5 cm, 0.6 cm, 0.7 cm, 0.8 cm, 0.9 cm, 1.0 cm, 1.1 cm, 1.2 cm, 1.3 cm, 1.4 cm, 1.5 cm, 1.6 cm, 1.7 cm, 1.8 cm, 1.9 cm, 2.0 cm, 3.0 cm, 4.0 cm, 5.0 cm, 6.0 cm, 7.0 cm, 8.0 cm, 9.0 cm, or 10.0 cm. In some embodiments, the treatment, pretreatment, process, or preprocess is physical, chemical, mechanical, electrical, or biological, or any combination thereof.

In some embodiments, the at least one algae are not treated or not pretreated or not processed or not preprocessed to reduce the size or particle size or of the at least one algae. In some embodiments, the at least one algae are optionally treated or optionally pretreated or optionally processed or optionally preprocessed to reduce the size or particle size or of the at least one algae.

In some embodiments, the at least one macroalgae are not treated or not pretreated or not processed or not preprocessed to reduce the size or particle size or of the at least one macroalgae. In some embodiments, the at least one macroalgae are optionally treated or optionally pretreated or optionally processed or optionally preprocessed to reduce the size or particle size or of the at least one macroalgae.

In some embodiments, the at least one microalgae are not treated or not pretreated or not processed or not preprocessed to reduce the size or particle size or of the at least one microalgae. In some embodiments, the at least one microalgae are treated or pretreated or processed or preprocessed to reduce the size or particle size or of the at least one microalgae. In some embodiments, the at least one microalgae are optionally treated or optionally pretreated or optionally processed or optionally preprocessed to reduce the size or particle size or of the at least one microalgae. In some embodiments, the treatment, pretreatment, process, or preprocess is physical, chemical, mechanical, electrical, or biological, or any combination thereof. In some embodiments, the reduced size of at least one microalgae is 0.1 um to less than 700 um, or 0.1 um to 699 um, or 0.1 um to 600 um, or 0.1 um to 500 um, or 0.1 um to 400 um, or 0.1 um to 300 um, or 0.1 um to 200 um, or 0.1 um to 100 um, or 0.1 um to 50 um, or any size between these ranges. In some embodiments, the size or particle size of the at least one microalgae is reduced relative to or compared to the native size or original size of the at least one microalgae before being treated or pretreated or processed or preprocessed to reduce the size or particle size of the at least one microalgae. In some embodiments, the size or particle size of the at least one microalgae is reduced relative to or compared to the size or particle size of the at least one microalgae before being treated or pretreated or processed or preprocessed to reduce the size or particle size of the at least one microalgae.

In some embodiments, the present invention provides a method for cultivating at least one fungal mycelium or at least one mushroom, the method comprising: providing a substrate of the present invention; inoculating the substrate with fungal spores or fungal mycelium; and applying conditions to stimulate growth of at least one fungal mycelium or at least one mushroom.

In some embodiments, the present invention provides a method for cultivating at least one fungal mycelium or at least one mushroom, the method comprising: providing a substrate of the present invention; inoculating the substrate with spores or mycelium; and applying conditions to stimulate growth of at least one fungal mycelium or at least one mushroom.

In some embodiments, the present invention provides a method for cultivating at least one mycelium or at least one mushroom, the method comprising: providing a substrate of the present invention; inoculating the substrate with spores or mycelium; and applying conditions to stimulate growth of at least one mycelium or at least one mushroom.

In some embodiments, the present invention provides a method for cultivating at least one fungal mycelium, the method comprising: providing a substrate of the present invention; inoculating the substrate with fungal spores or fungal mycelium; and applying conditions to stimulate growth of at least one fungal mycelium.

In some embodiments, the present invention provides a method for cultivating at least one fungal mycelium, the method comprising: providing a substrate of the present invention; inoculating the substrate with spores or mycelium; and applying conditions to stimulate growth of at least one fungal mycelium.

In some embodiments, the present invention provides a method for cultivating at least one mycelium, the method comprising: providing a substrate of the present invention; inoculating the substrate with spores or mycelium; and applying conditions to stimulate growth of at least one mycelium.

In some embodiments, the present invention provides a method for cultivating at least one mushroom, the method comprising: providing a substrate of the present invention; inoculating the substrate with fungal spores or fungal mycelium; and applying conditions to stimulate growth of at least one mushroom.

In some embodiments, the present invention provides a method for cultivating at least one mushroom, the method comprising: providing a substrate of the present invention; inoculating the substrate with spores or mycelium; and applying conditions to stimulate growth of at least one mushroom.

A method for cultivating at least one fungal mycelium or at least one mushroom and for producing algal biomass, the method comprising: providing a substrate of the present invention; inoculating the substrate with fungal spores or fungal mycelium; applying conditions to stimulate growth of at least one fungal mycelium or at least one mushroom; capturing carbon dioxide produced during the cultivation of the at least one fungal mycelium or the at least one mushroom; converting the captured carbon dioxide into sodium bicarbonate; and utilizing the sodium bicarbonate as a carbon source to produce algal biomass.

A method for cultivating at least one fungal mycelium or at least one mushroom and for producing algal biomass, the method comprising: providing a substrate of the present invention; inoculating the substrate with spores or mycelium; applying conditions to stimulate growth of at least one fungal mycelium or at least one mushroom; capturing carbon dioxide produced during the cultivation of the at least one fungal mycelium or the at least one mushroom; converting the captured carbon dioxide into sodium bicarbonate; and utilizing the sodium bicarbonate as a carbon source to produce algal biomass.

A method for cultivating at least one fungal mycelium and for producing algal biomass, the method comprising: providing a substrate of the present invention; inoculating the substrate with fungal spores or fungal mycelium; applying conditions to stimulate growth of at least one fungal mycelium; capturing carbon dioxide produced during the cultivation of the at least one fungal mycelium; converting the captured carbon dioxide into sodium bicarbonate; and utilizing the sodium bicarbonate as a carbon source to produce algal biomass.

A method for cultivating at least one fungal mycelium and for producing algal biomass, the method comprising: providing a substrate of the present invention; inoculating the substrate with spores or mycelium; applying conditions to stimulate growth of at least one fungal mycelium; capturing carbon dioxide produced during the cultivation of the at least one fungal mycelium; converting the captured carbon dioxide into sodium bicarbonate; and utilizing the sodium bicarbonate as a carbon source to produce algal biomass.

A method for cultivating at least one mycelium and for producing algal biomass, the method comprising: providing a substrate of the present invention; inoculating the substrate with spores or mycelium; applying conditions to stimulate growth of at least one mycelium; capturing carbon dioxide produced during the cultivation of the at least one mycelium; converting the captured carbon dioxide into sodium bicarbonate; and utilizing the sodium bicarbonate as a carbon source to produce algal biomass.

A method for cultivating at least one mushroom and for producing algal biomass, the method comprising: providing a substrate of the present invention; inoculating the substrate with fungal spores or fungal mycelium; applying conditions to stimulate growth of at least one mushroom; capturing carbon dioxide produced during the cultivation of the at least one mushroom; converting the captured carbon dioxide into sodium bicarbonate; and utilizing the sodium bicarbonate as a carbon source to produce algal biomass.

A method for cultivating at least one mushroom and for producing algal biomass, the method comprising: providing a substrate of the present invention; inoculating the substrate with spores or mycelium; applying conditions to stimulate growth of at least one mushroom; capturing carbon dioxide produced during the cultivation of the at least one mushroom; converting the captured carbon dioxide into sodium bicarbonate; and utilizing the sodium bicarbonate as a carbon source to produce algal biomass.

A system for cultivating at least one fungal mycelium or at least one mushroom and for producing algal biomass, the system comprising: a first chamber, wherein the first chamber is adapted for cultivating at least one fungal mycelium or at least one mushroom and capturing carbon dioxide produced during cultivation of the at least one fungal mycelium or the at least one mushroom, and wherein the first chamber comprises a substrate of the present invention; a second chamber, wherein the second chamber is in communication with the first chamber, and wherein the second chamber is adapted to convert the captured carbon dioxide into sodium bicarbonate; and a third chamber, wherein the third chamber is in communication with the second chamber, and wherein the third chamber is adapted for using the sodium bicarbonate as a carbon source for producing algal biomass.

A system for cultivating at least one fungal mycelium and for producing algal biomass, the system comprising: a first chamber, wherein the first chamber is adapted for cultivating at least one fungal mycelium and capturing carbon dioxide produced during cultivation of the at least one fungal mycelium, and wherein the first chamber comprises a substrate of the present invention; a second chamber, wherein the second chamber is in communication with the first chamber, and wherein the second chamber is adapted to convert the captured carbon dioxide into sodium bicarbonate; and a third chamber, wherein the third chamber is in communication with the second chamber, and wherein the third chamber is adapted for using the sodium bicarbonate as a carbon source for producing algal biomass.

A system for cultivating at least one mycelium and for producing algal biomass, the system comprising: a first chamber, wherein the first chamber is adapted for cultivating at least one mycelium and capturing carbon dioxide produced during cultivation of the at least one mycelium, and wherein the first chamber comprises a substrate of the present invention; a second chamber, wherein the second chamber is in communication with the first chamber, and wherein the second chamber is adapted to convert the captured carbon dioxide into sodium bicarbonate; and a third chamber, wherein the third chamber is in communication with the second chamber, and wherein the third chamber is adapted for using the sodium bicarbonate as a carbon source for producing algal biomass.

A system for cultivating at least one mushroom and for producing algal biomass, the system comprising: a first chamber, wherein the first chamber is adapted for cultivating at least one mushroom and capturing carbon dioxide produced during cultivation of the at least one mushroom, and wherein the first chamber comprises a substrate of the present invention; a second chamber, wherein the second chamber is in communication with the first chamber, and wherein the second chamber is adapted to convert the captured carbon dioxide into sodium bicarbonate; and a third chamber, wherein the third chamber is in communication with the second chamber, and wherein the third chamber is adapted for using the sodium bicarbonate as a carbon source for producing algal biomass.

In some embodiments, the at least one algae is not Spirulina maxima or Chlorella vulgaris. In some embodiments, the at least one algae is not Spirulina maxima, Chlorella vulgaris, or Arthrospira maxima. In some embodiments, the at least one algae is not from the genus Chlorella or the genus Spirulina. In some embodiments, the at least one algae is not from the genus Chlorella, or the genus Spirulina, or the genus Arthrospira.

In some embodiments, an amount of at least one algae in the substrate is about 1% to about 15% by weight of the total weight of the substrate. In some embodiments, the amount of at least one algae in the substrate is 1% to 15% by weight of the total weight of the substrate. In some embodiments, the amount of at least one algae in the substrate is greater than 0.1% by weight of the total weight of the substrate. In some embodiments, an amount of at least one algae in the substrate is 0.1% to 100% by weight of the total weight of the substrate. In some embodiments, the amount of at least one algae in the substrate is greater than 0.001% by weight of the total weight of the substrate. In some embodiments, an amount of at least one algae in the substrate is 0.001% to 100% by weight of the total weight of the substrate. In some embodiments, an amount of at least one algae in the substrate is 1% to 100% by weight of the total weight of the substrate.

In some embodiments, an amount of at least one algae in the substrate is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 percent by weight of the total weight of the substrate.

In some embodiments, an amount of at least one microalgae in the substrate is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 percent by weight of the total weight of the substrate.

In some embodiments, an amount of at least one macroalgae in the substrate is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 percent by weight of the total weight of the substrate.

In some embodiments, the at least one algae is single-celled green algae. In some embodiments, the at least one algae is at least one microphyte.

In some embodiments, the at least one algae is from the genus Chlorella, or the genus Spirulina, or combination thereof. In some embodiments the at least one algae is from the genus Chlorella, the genus Spirulina, or the genus Arthrospira, or combination thereof.

In some embodiments, the at least one algae from the genus Chlorella is Chlorella autotrophica, Chlorella coloniales, Chlorella lewinii, Chlorella minutissima, Chlorella pituita, Chlorella pulchelloides, Chlorella pyrenoidosa, Chlorella rotunda, Chlorella singularis, Chlorella sorokiniana, Chlorella variabilis, Chlorella volutis, or Chlorella vulgaris, or any combination thereof. In some embodiments, the at least one algae from the genus Chlorella is Chlorella autotrophica, Chlorella coloniales, Chlorella lewinii, Chlorella minutissima, Chlorella pituita, Chlorella pulchelloides, Chlorella pyrenoidosa, Chlorella rotunda, Chlorella singularis, Chlorella sorokiniana, Chlorella variabilis, or Chlorella volutis, or any combination thereof. In some embodiments, the at least one algae from the genus Chlorella is not Chlorella vulgaris.

In some embodiments, the at least one algae is Chlorella autotrophica, Chlorella coloniales, Chlorella lewinii, Chlorella minutissima, Chlorella pituita, Chlorella pulchelloides, Chlorella pyrenoidosa, Chlorella rotunda, Chlorella singularis, Chlorella sorokiniana, Chlorella variabilis, Chlorella volutis, or Chlorella vulgaris, or any combination thereof. In some embodiments, the at least one algae is Chlorella autotrophica, Chlorella coloniales, Chlorella lewinii, Chlorella minutissima, Chlorella pituita, Chlorella pulchelloides, Chlorella pyrenoidosa, Chlorella rotunda, Chlorella singularis, Chlorella sorokiniana, Chlorella variabilis, or Chlorella volutis, or any combination thereof. In some embodiments, the at least one algae is not Chlorella vulgaris.

In some embodiments, the at least one algae from the genus Spirulina is Arthrospira platensis, Arthrospira fusiformis, or Arthrospira maxima, or any combination thereof.

In some embodiments, the at least one algae from the genus Spirulina is Arthrospira platensis, or Arthrospira fusiformis, or any combination thereof. In some embodiments, the at least one algae from the genus Spirulina is not Arthrospira maxima.

In some embodiments, the at least one algae from the genus Arthrospira is Arthrospira platensis, Arthrospira fusiformis, or Arthrospira maxima, or any combination thereof. In some embodiments, the at least one algae from the genus Arthrospira is Arthrospira platensis, or Arthrospira fusiformis, or any combination thereof. In some embodiments, the at least one algae from the genus Arthrospira is not Arthrospira maxima.

In some embodiments, the at least one algae is Arthrospira platensis, Arthrospira fusiformis, or Arthrospira maxima, or any combination thereof. In some embodiments, the at least one algae is Arthrospira platensis, or Arthrospira fusiformis, or any combination thereof. In some embodiments, the at least one algae is not Arthrospira maxima.

In some embodiments, the algae is red algae, brown algae, or green algae, or any combination thereof.

In some embodiments, the red algae is Rhodophyta, the brown algae is Phaeophyta, and the green algae is Chlorophyta.

In some embodiments, the algae is from the class Phaeophyceae.

In some embodiments, the at least one algae is seaweed. In some embodiments, the at least one algae is edible seaweed. In some embodiments, the at least one algae is non-edible seaweed.

In some embodiments, the substrate further comprises lignocellulosic biomass. In some embodiments, a weight ratio of the at least one algae to the lignocellulosic biomass is 1:1, 2:1; 3:1, 4:1; 5:1, 6:1, 7:1, 8:1, 9:1, 10:1, 20:1, 30:1; 40:1, or 50:1. In some embodiments, a weight ratio of the at least one algae to the lignocellulosic biomass is 1:1, 1:2, 1:3, 1:4, 1:5, 1:6; 1:7; 1:8, 1:9, 1:10; 1:20; 1:30, 1:40, or 1:50. In some embodiments, a weight ratio of the at least one algae to the lignocellulosic biomass is 50:50. In some embodiments, the lignocellulosic biomass is wood, straw, agricultural waste, wheat bran, or corn flour, or any combination thereof. In some embodiments, the lignocellulosic biomass is wood, straw, agricultural waste, or wheat bran, or any combination thereof.

In some embodiments, the lignocellulosic biomass is hay, seed hulls, saw dust, plant cake, plant extract cake, leaves, fruit pomaces, nut shells, straw, seed husk, grain husk, or bran, or any combination thereof.

In some embodiments, the lignocellulosic biomass is alfalfa hay, timothy hay, oat hay, clover hay, peanut hull, cotton seed hull, sunflower seed hull, oat hull, oak saw dust, pine saw dust, poplar saw dust, peanut cake, olive oil cake, oil palm cake, date palm leaves, tea leaves, banana leaves, apple pomace, tomato pomace, almond shell, walnut shell, wheat straw, rice straw, rice husk, coffee bean husk, rice bran, or wheat bran, or any combination thereof.

In some embodiments, the lignocellulosic biomass is alfalfa hay, timothy hay, oat hay, clover hay, peanut hull, cotton seed hull, sunflower seed hull, oat hull, oak saw dust, pine saw dust, poplar saw dust, peanut cake, olive oil cake, oil palm cake, date palm leaves, tea leaves, banana leaves, apple pomace, tomato pomace, almond shell, walnut shell, wheat straw, rice straw, rice husk, coffee bean husk, rice bran, wheat bran, cocoa pod waste, coconut coir, coffee bean extract, corn flour, corn stover, date seeds, sorghum stubbles, sugarcane bagasse, or switchgrass, or any combination thereof.

In some embodiments the lignocellulosic biomass is composted lignocellulosic biomass. In some embodiments, the composted lignocellulosic biomass is agricultural waste.

In some embodiments, the at least one algae is at least one microalgae, or at least one macroalgae, or combination thereof. In some embodiments, the at least one algae is at least one microalgae. In some embodiments, the at least one algae is at least one macroalgae. In some embodiments, the at least one microalgae is not Spirulina maxima or Chlorella vulgaris. In some embodiments, the at least one microalgae is not Spirulina maxima, Chlorella vulgaris, or Arthrospira maxima. In some embodiments, the at least one microalgae is not from the genus Chlorella or the genus Spirulina. In some embodiments, the at least one microalgae is not from the genus Chlorella, or the genus Spirulina, or the genus Arthrospira.

In some embodiments, the at least one microalgae is single-celled green algae. In some embodiments, the at least one microalgae is at least one microphyte.

In some embodiments, the at least one microalgae is from the genus Chlorella, or the genus Spirulina, or combination thereof. In some embodiments the at least one microalgae is from the genus Chlorella, the genus Spirulina, or the genus Arthrospira, or combination thereof.

In some embodiments, the at least one microalgae from the genus Chlorella is Chlorella autotrophica, Chlorella coloniales, Chlorella lewinii, Chlorella minutissima, Chlorella pituita, Chlorella pulchelloides, Chlorella pyrenoidosa, Chlorella rotunda, Chlorella singularis, Chlorella sorokiniana, Chlorella variabilis, Chlorella volutis, or Chlorella vulgaris, or any combination thereof. In some embodiments, the at least one microalgae from the genus Chlorella is Chlorella autotrophica, Chlorella coloniales, Chlorella lewinii, Chlorella minutissima, Chlorella pituita, Chlorella pulchelloides, Chlorella pyrenoidosa, Chlorella rotunda, Chlorella singularis, Chlorella sorokiniana, Chlorella variabilis, or Chlorella volutis, or any combination thereof. In some embodiments, the at least one microalgae from the genus Chlorella is not Chlorella vulgaris.

In some embodiments, the at least one microalgae is Chlorella autotrophica, Chlorella coloniales, Chlorella lewinii, Chlorella minutissima, Chlorella pituita, Chlorella pulchelloides, Chlorella pyrenoidosa, Chlorella rotunda, Chlorella singularis, Chlorella sorokiniana, Chlorella variabilis, Chlorella volutis, or Chlorella vulgaris, or any combination thereof. In some embodiments, the at least one microalgae is Chlorella autotrophica, Chlorella coloniales, Chlorella lewinii, Chlorella minutissima, Chlorella pituita, Chlorella pulchelloides, Chlorella pyrenoidosa, Chlorella rotunda, Chlorella singularis, Chlorella sorokiniana, Chlorella variabilis, or Chlorella volutis, or any combination thereof. In some embodiments, the at least one microalgae is not Chlorella vulgaris.

In some embodiments, the at least one microalgae from the genus Spirulina is Arthrospira platensis, Arthrospira fusiformis, or Arthrospira maxima, or any combination thereof.

In some embodiments, the at least one microalgae from the genus Spirulina is Arthrospira platensis, or Arthrospira fusiformis, or any combination thereof. In some embodiments, the at least one microalgae from the genus Spirulina is not Arthrospira maxima.

In some embodiments, the at least one microalgae from the genus Arthrospira is Arthrospira platensis, Arthrospira fusiformis, or Arthrospira maxima, or any combination thereof. In some embodiments, the at least one microalgae from the genus Arthrospira is Arthrospira platensis, or Arthrospira fusiformis, or any combination thereof. In some embodiments, the at least one microalgae from the genus Arthrospira is not Arthrospira maxima.

In some embodiments, the at least one microalgae is Arthrospira platensis, Arthrospira fusiformis, Arthrospira maxima, or any combination thereof. In some embodiments, the at least one microalgae is Arthrospira platensis, or Arthrospira fusiformis, or any combination thereof. In some embodiments, the at least one microalgae is not Arthrospira maxima.

In some embodiments, the at least one macroalgae is seaweed. In some embodiments, the at least one macroalgae is edible seaweed. In some embodiments, the at least one macroalgae is non-edible seaweed.

In some embodiments, the at least one macroalgae is sea lettuce, or sea moss, or a combination thereof.

In some embodiments, the sea lettuce is Ulva lactuca.

In some embodiments, the sea moss is Chondrus crispus.

In some embodiments, the at least one macroalgae is nori, bladderwrack, Irish moss, kelp, dulse, or wakame, or any combination thereof.

In some embodiments, the nori is from the genus Pyropia. In some embodiments, the nori from the genus Pyropia is Pyropia yezoensis, or Pyropia tenera, or any combination thereof. In some embodiments the nori is Pyropia yezoensis, or Pyropia tenera, or any combination thereof.

In some embodiments, the bladderwrack is Fucus vesiculosus.

In some embodiments, the Irish moss is Chondrus crispus.

In some embodiments, the kelp is from the genus Laminaria. In some embodiments, the kelp from the genus Laminaria is Laminaria agardhii, Laminaria bongardina, Laminaria cuneifolia, Laminaria dentigera, Laminaria digitata, Laminaria ephemera, Laminaria farlowii, Laminaria groenlandica, Laminaria longicruris, Laminaria nigripes, Laminaria ontermedia, Laminaria pallida, Laminaria platymeris, Laminaria saccharina, Laminaria setchellii, Laminaria sinclairii, Laminaria solidungula, or Laiminaria stenophylla, or any combination thereof.

In some embodiments, the kelp is Laminaria agardhii, Laminaria bongardina, Laminaria cuneifolia, Laminaria dentigera, Laminaria digitata, Laminaria ephemera, Laminaria farlowii, Laminaria groenlandica, Laminaria longicruris, Laminaria nigripes, Laminaria ontermedia, Laminaria pallida, Laminaria platymeris, Laminaria saccharina, Laminaria setchellii, Laminaria sinclairii, Laminaria solidungula, or Laiminaria stenophylla, or any combination thereof.

In some embodiments, the kelp is bull kelp, giant kelp, or kombu, or any combination thereof.

In some embodiments, the kelp is Nereocystis luetkeana, Macrocystis pyrifera, or Saccharina japonica, or any combination thereof.

In some embodiments, the kelp is Laminaria digitata, Laminaria hyperborea, Laminaria ochroleuca, or Saccharina latissima, or any combination thereof.

In some embodiments, the kelp is from the order Laminariales.

In some embodiments, the kelp is kelp extract.

In some embodiments, the dulse is from the genus Palmaria.

In some embodiments, the dulse is Palmaria palmata.

In some embodiments, the wakame is from the genus Undaria.

In some embodiments, the wakame is Undaria pinnatifida.

In some embodiments, the macroalgae is red macroalgae, brown macroalgae, or green macroalgae, or any combination thereof.

In some embodiments, the red macroalgae is Rhodophyta, the brown macroalgae is Phaeophyta, and the green macroalgae is Chlorophyta.

In some embodiments, the fungus is at least one mushroom, at least one yeast, or at least one mold, or any combination thereof. In some embodiments, the at least one fungus is at least one mushroom. In some embodiments, the fungus is a mushroom. In some embodiments, the fungus is at least one yeast. In some embodiments, the fungus is at least one mold. In some embodiments, the fungus is not a yeast. In some embodiments, the fungus is not a mold. In some embodiments, the fungus is not a yeast, and the fungus is not a mold.

In some embodiments, the fungus is at least one mushroom species. In some embodiments, the at least one mushroom species is Hypsizygus ulmarius, Calocybe indica, Pleurotus ostreatus, Ganoderma lucidum, or Agaricus bisporus, or any combination thereof.

In some embodiments, the fungus is a fungus order, wherein the fungus order is Polyporales, Agaricales, Russulales, or Pezizales, or any combination thereof.

In some embodiments, the fungus is Ganoderma lucidum, Grifola frondosa, Fomes fomentarius, Daedaleopsis confragosa, Lentinula edodes, Pleurotus ostreatus, Calocybe indica, Agrocybe aegerita, Hericium erinaceus, or Morchella angusticeps, or any combination thereof.

In some embodiments, the at least one mushroom is Hypsizygus ulmarius, Calocybe indica, Pleurotus ostreatus, Ganoderma lucidum, or Agaricus bisporus.

In some embodiments, the at least one mushroom is Ganoderma lucidum, Grifola frondosa, Fomes fomentarius, Daedaleopsis confragosa, Lentinula edodes, Pleurotus ostreatus, Calocybe indica, Agrocybe aegerita, Hericium erinaceus, or Morchella angusticeps, or any combination thereof.

In some embodiments, the at least one mushroom is Hypsizygus ulmarius, Calocybe indica, Pleurotus ostreatus, Ganoderma lucidum, Agaricus bisporus, Ganoderma lucidum, Grifola frondosa, Fomes fomentarius, Daedaleopsis confragosa, Lentinula edodes, Pleurotus ostreatus, Agrocybe aegerita, Hericium erinaceus, or Morchella angusticeps, or any combination thereof.

In some embodiments, the at least one cyanobacteria is not Spirulina maxima. In some embodiments, the at least one cyanobacteria is not Arthrospira maxima. In some embodiments, the at least one cyanobacteria is not Spirulina maxima or Arthrospira maxima. In some embodiments, the at least one cyanobacteria is not from the genus Spirulina. In some embodiments, the at least one cyanobacteria is not from the genus Arthrospira. In some embodiments, the at least one cyanobacteria is not from the genus Spirulina, or the genus Arthrospira.

In some embodiments, the at least one cyanobacteria from the genus Spirulina is Arthrospira platensis, Arthrospira fusiformis, or Arthrospira maxima, or any combination thereof. In some embodiments, the at least one cyanobacteria from the genus Spirulina is Arthrospira platensis, or Arthrospira fusiformis, or any combination thereof. In some embodiments, the at least one cyanobacteria from the genus Spirulina is not Arthrospira maxima.

In some embodiments, the at least one cyanobacteria from the genus Spirulina is Spirulina maxima. In some embodiments, the at least one cyanobacteria from the genus Spirulina is not Spirulina maxima.

In some embodiments, the at least one cyanobacteria is Spirulina maxima. In some embodiments, the at least one cyanobacteria is not Spirulina maxima.

In some embodiments, the at least one cyanobacteria from the genus Arthrospira is Arthrospira platensis, Arthrospira fusiformis, or Arthrospira maxima, or any combination thereof. In some embodiments, the at least one cyanobacteria from the genus Arthrospira is Arthrospira platensis, or Arthrospira fusiformis, or any combination thereof. In some embodiments, the at least one cyanobacteria from the genus Arthrospira is not Arthrospira maxima.

In some embodiments, the at least one cyanobacteria is Arthrospira platensis, Arthrospira fusiformis, or Arthrospira maxima, or any combination thereof. In some embodiments, the at least one cyanobacteria is Arthrospira platensis, or Arthrospira fusiformis, or any combination thereof. In some embodiments, the at least one cyanobacteria is not Arthrospira maxima.

Additional embodiments include the following:

Embodiment 46. A substrate for cultivating at least one fungal mycelium or at least one mushroom, the substrate comprising: at least one algae, provided that the at least one algae is not Spirulina maxima or Chlorella vulgaris.

Embodiment 47. The substrate of embodiment 46, wherein the at least one algae has been treated to remove at least a portion of salt from the at least one algae.

Embodiment 48. The substrate of embodiment 46, wherein the substrate further comprises lignocellulosic biomass.

Embodiment 49. The substrate of embodiment 46, wherein the at least one algae is at least one microalgae, or at least one macroalgae, or combination thereof, provided that the at least one microalgae is not Spirulina maxima or Chlorella vulgaris.

Embodiment 50. The substrate of embodiment 49, wherein the at least one macroalgae is nori, bladderwrack, Irish moss, kelp, dulse, or wakame, or any combination thereof.

Embodiment 51. The substrate of embodiment 46, wherein the at least one fungal mycelium or the at least one mushroom is Hypsizygus ulmarius, Calocybe indica, Pleurotus ostreatus, Ganoderma lucidum, Agaricus bisporus, Ganoderma lucidum, Grifola frondosa, Fomes fomentarius, Daedaleopsis confragosa, Lentinula edodes, Pleurotus ostreatus, Agrocybe aegerita, Hericium erinaceus, or Morchella angusticeps, or any combination thereof.

Embodiment 52. A method for cultivating at least one fungal mycelium or at least one mushroom, the method comprising: providing a substrate of embodiment 46; inoculating the substrate with fungal spores or fungal mycelium; and applying conditions to stimulate growth of at least one fungal mycelium or at least one mushroom.

Embodiment 53. The method of embodiment 52, wherein the substrate further comprises lignocellulosic biomass.

Embodiment 54. The method of embodiment 52, wherein the at least one algae is at least one microalgae, or at least one macroalgae, or combination thereof, provided that the at least one microalgae is not Spirulina maxima or Chlorella vulgaris.

Embodiment 55. The method of embodiment 54, wherein the at least one macroalgae is nori, bladderwrack, Irish moss, kelp, dulse, or wakame, or any combination thereof.

Embodiment 56. A method for cultivating at least one fungal mycelium or at least one mushroom and for producing algal biomass, the method comprising: providing a substrate of embodiment 46; inoculating the substrate with fungal spores or fungal mycelium; applying conditions to stimulate growth of at least one fungal mycelium or at least one mushroom; capturing carbon dioxide produced during the cultivation of the at least one fungal mycelium or the at least one mushroom; converting the captured carbon dioxide into sodium bicarbonate; and utilizing the sodium bicarbonate as a carbon source to produce algal biomass.

Embodiment 57. The method of embodiment 56, wherein the substrate further comprises lignocellulosic biomass.

Embodiment 58. The method of embodiment 56, wherein the at least one algae is at least one microalgae, or at least one macroalgae, or combination thereof, provided that the at least one microalgae is not Spirulina maxima or Chlorella vulgaris.

Embodiment 59. The method of embodiment 58, wherein the at least one macroalgae is nori, bladderwrack, Irish moss, kelp, dulse, or wakame, or any combination thereof.

Embodiment 60. A system for cultivating at least one fungal mycelium or at least one mushroom and for producing algal biomass, the system comprising: a first chamber, wherein the first chamber is adapted for cultivating at least one fungal mycelium or at least one mushroom and capturing carbon dioxide produced during cultivation of the at least one fungal mycelium or the at least one mushroom, and wherein the first chamber comprises a substrate of embodiment 46; a second chamber, wherein the second chamber is in communication with the first chamber, and wherein the second chamber is adapted to convert the captured carbon dioxide into sodium bicarbonate; and a third chamber, wherein the third chamber is in communication with the second chamber, and wherein the third chamber is adapted for using the sodium bicarbonate as a carbon source for producing algal biomass.

Embodiment 61. The system of embodiment 60, wherein the substrate further comprises lignocellulosic biomass.

Embodiment 62. The system of embodiment 60, wherein the at least one algae is at least one microalgae, or at least one macroalgae, or combination thereof, provided that the at least one microalgae is not Spirulina maxima or Chlorella vulgaris.

Embodiment 63. The system of embodiment 62, wherein the at least one macroalgae is nori, bladderwrack, Irish moss, kelp, dulse, or wakame, or any combination thereof.

Embodiment 64. The system of embodiment 60, wherein the third chamber comprises algal biomass.

Embodiment 65. The system of embodiment 60, wherein the at least one mushroom is Hypsizygus ulmarius, Calocybe indica, Pleurotus ostreatus, Ganoderma lucidum, Agaricus bisporus, Ganoderma lucidum, Grifola frondosa, Fomes fomentarius, Daedaleopsis confragosa, Lentinula edodes, Pleurotus ostreatus, Agrocybe aegerita, Hericium erinaceus, or Morchella angusticeps, or any combination thereof.

Embodiment 66. The system of claim 60, wherein the system further comprises a fourth chamber, wherein the fourth chamber is adapted for storing algal biomass.

Additional embodiments include the following:

Embodiment 67. A substrate for cultivating at least one fungal mycelium or at least one mushroom, the substrate comprising: at least one algae, wherein the at least one algae has been treated to remove at least a portion of salt from the at least one algae, and provided that the at least one algae is not Spirulina maxima or Chlorella vulgaris.

Embodiment 68. The substrate of embodiment 67, wherein the substrate further comprises lignocellulosic biomass.

Embodiment 69. The substrate of embodiment 67, wherein the at least one algae is at least one microalgae, or at least one macroalgae, or combination thereof, provided that the at least one microalgae is not Spirulina maxima or Chlorella vulgaris.

Embodiment 70. The substrate of embodiment 69, wherein the at least one macroalgae is nori, bladderwrack, Irish moss, kelp, dulse, or wakame, or any combination thereof.

Embodiment 71. The substrate of embodiment 67, wherein the at least one fungal mycelium or the at least one mushroom is Hypsizygus ulmarius, Calocybe indica, Pleurotus ostreatus, Ganoderma lucidum, Agaricus bisporus, Ganoderma lucidum, Grifola frondosa, Fomes fomentarius, Daedaleopsis confragosa, Lentinula edodes, Pleurotus ostreatus, Agrocybe aegerita, Hericium erinaceus, or Morchella angusticeps, or any combination thereof.

Embodiment 72. A method for cultivating at least one fungal mycelium or at least one mushroom, the method comprising: providing a substrate of embodiment 67; inoculating the substrate with fungal spores or fungal mycelium; and applying conditions to stimulate growth of at least one fungal mycelium or at least one mushroom.

Embodiment 73. The method of embodiment 72, wherein the substrate further comprises lignocellulosic biomass.

Embodiment 74. The method of embodiment 72, wherein the at least one algae is at least one microalgae, or at least one macroalgae, or combination thereof, provided that the at least one microalgae is not Spirulina maxima or Chlorella vulgaris.

Embodiment 75. The method of embodiment 74, wherein the at least one macroalgae is nori, bladderwrack, Irish moss, kelp, dulse, or wakame, or any combination thereof.

Embodiment 76. A method for cultivating at least one fungal mycelium or at least one mushroom and for producing algal biomass, the method comprising: providing a substrate of embodiment 67; inoculating the substrate with fungal spores or fungal mycelium; applying conditions to stimulate growth of at least one fungal mycelium or at least one mushroom; capturing carbon dioxide produced during the cultivation of the at least one fungal mycelium or the at least one mushroom; converting the captured carbon dioxide into sodium bicarbonate; and utilizing the sodium bicarbonate as a carbon source to produce algal biomass.

Embodiment 77. The method of embodiment 76, wherein the substrate further comprises lignocellulosic biomass.

Embodiment 78. The method of embodiment 76, wherein the at least one algae is at least one microalgae, or at least one macroalgae, or combination thereof, provided that the at least one microalgae is not Spirulina maxima or Chlorella vulgaris.

Embodiment 79. The method of embodiment 78, wherein the at least one macroalgae is nori, bladderwrack, Irish moss, kelp, dulse, or wakame, or any combination thereof.

Embodiment 80. A system for cultivating at least one fungal mycelium or at least one mushroom and for producing algal biomass, the system comprising: a first chamber, wherein the first chamber is adapted for cultivating at least one fungal mycelium or at least one mushroom and capturing carbon dioxide produced during cultivation of the at least one fungal mycelium or the at least one mushroom, and wherein the first chamber comprises a substrate of embodiment 67; a second chamber, wherein the second chamber is in communication with the first chamber, and wherein the second chamber is adapted to convert the captured carbon dioxide into sodium bicarbonate; and a third chamber, wherein the third chamber is in communication with the second chamber, and wherein the third chamber is adapted for using the sodium bicarbonate as a carbon source for producing algal biomass.

Embodiment 81. The system of embodiment 80, wherein the substrate further comprises lignocellulosic biomass.

Embodiment 82. The system of embodiment 80, wherein the at least one algae is at least one microalgae, or at least one macroalgae, or combination thereof, provided that the at least one microalgae is not Spirulina maxima or Chlorella vulgaris.

Embodiment 83. The system of embodiment 82, wherein the at least one macroalgae is nori, bladderwrack, Irish moss, kelp, dulse, or wakame, or any combination thereof.

Embodiment 84. The system of embodiment 80, wherein the third chamber comprises algal biomass.

Embodiment 85. The system of embodiment 80, wherein the at least one mushroom is Hypsizygus ulmarius, Calocybe indica, Pleurotus ostreatus, Ganoderma lucidum, Agaricus bisporus, Ganoderma lucidum, Grifola frondosa, Fomes fomentarius, Daedaleopsis confragosa, Lentinula edodes, Pleurotus ostreatus, Agrocybe aegerita, Hericium erinaceus, or Morchella angusticeps, or any combination thereof.

Embodiment 86. The system of claim 80, wherein the system further comprises a fourth chamber, wherein the fourth chamber is adapted for storing algal biomass.

### EXAMPLES

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

### Experimental Examples

In this experiment, various types of micro- and macroalgae were evaluated as potential substrates for mycelium growth. The study aimed to explore the suitability of different algae species as a substrate for mushroom cultivation, focusing on the impact of algae on the growth and development of mycelium. Microalgae such as Chlorella and Spirulina were used, either alone or in combination, and were evaluated. Macroalgae, such as dulse, nori, wakame, kelp extract, Irish moss, and bladderwrack was evaluated. These species were selected for their high nutritional value and potential suitability as substrates for mushroom cultivation. Mycelium requires a range of nutrients, including nitrogen, phosphorus, potassium, amino acids, and lipids to grow and develop properly. All algae are rich in these essential nutrients, as well as other micronutrients like vitamins and minerals that are important for mycelial growth. Table 1 shows the chemical composition of lignocellulosic biomass, micro- and macroalgae.

TABLE 1
Chemical composition of lignocellulosic biomass, micro, and macroalgae.

| Chemical | | | Macro- | | Wheat | Corn |
|---|---|---|---|---|---|---|
| composition | Spirulina | Chlorella | algae | Straw | bran | flour |
| Protein | 55-70% | 50-60% | 5-35% | 2-6% | 12-18% | 9-10% |
| Carbohydrates | 15-25% | 15-20% | 30-60% | 70-90% | 60-70% | 25-30% |
| Lipids (fat) | 6-8% | 9-15% | 2-13% | 05-2% | 4-8% | 1-2% |
| Minerals | 5-7% | 5-10% | 0.5-5% | 0.01-0.2% | 0.1-2% | 6-10% |

Furthermore, spirulina and chlorella can be grown using raceway pond and tubular photobioreactor and harvest, making them a sustainable and cost-effective source of nutrients for used in this experiment. In this study, we evaluated the use of spirulina and chlorella algae, either alone or in combination, as a source of nutrients. Five different concentrations (1%, 5%, 10% and 15%) were added to DI water and agar for each algal species. For combination experiments equal amounts of both algae were combined in a ratio of 1:1. The study aimed to investigate the nutrient composition of these algae and their combination. Table 2 displays the quantities of ingredients utilized in the experiments.

TABLE 2
Media composition of algae-based media for Calocybe indica culturing.
Media combination

| | Agar powder | | Wet weight | Dry weight |
|---|---|---|---|---|
| Water (mL) | (g) | Algae % | algae (g) | algae (g) |
| 1000 | 20 | 1% | 10 | 9.04 |
| 1000 | 20 | 5% | 50 | 45.2 |
| 1000 | 20 | 10% | 100 | 90.4 |
| 1000 | 20 | 15% | 150 | 135.6 |

Based on the information presented in Table 2, the algae are combined with 1000 milliliters of deionized water and 20 grams of agar for each concentration. The use of agar in the mixture serves as a solidifying agent and helps to maintain the consistency of the solution.

Fungi have different pH preferences for optimal growth, with some species preferring a more acidic environment while others prefer a more alkaline environment. For many fungi, a slightly acidic pH range of 5.0 to 6.5 is considered optimal for growth and development. The pH level of the growing medium was set to 5.5, which is considered an optimal pH range for the growth of many types of fungi. This mixture is used in the experimental setup to study the effects of different concentrations on the growth of the algae. To ensure the sterility of media in microbiological experiments, autoclaving is a common technique employed. This process involves subjecting the media to high temperature and pressure conditions at 121° C. for a period of 30 minutes. To maintain sterility and prevent contamination of the experimental setup, the media is poured into petri dishes on a clean bench. By pouring the mixture into petri dishes within a clean bench, the risk of unintended microbial growth or contamination is minimized, ensuring that the experimental results are reliable and accurate. All experiments were performed in triplicate.

We have used Calocybe indica as a test fungus in our experiment while we are evaluating other edible fungus in the lab. The mushroom produced using Calocybe indica is commonly known as the milky mushroom which is edible and found in tropical and subtropical regions of the world. It is cultivated for its tasty and nutritious fruiting bodies, which are typically large, white, and milky in appearance. The milky mushroom has a high protein content, as well as various vitamins and minerals, making it a popular food item in many cultures. In addition to its culinary uses, Calocybe indica is also being studied for its potential medicinal properties. It is believed to have antioxidant, antimicrobial, and anti-inflammatory effects, among other health benefits. In this process, a small piece of milky white mushroom tissue, measuring 0.5 mm, is cut, and placed at the center of a petri dish that contains an algae-based growth medium. During the experiment, the samples were placed inside a growth chamber and maintained at a temperature of 32° C. for a period of 7-days. Throughout the experiment, pictures of the mycelium were taken at different time points. Specifically, photographs were captured on the day of culturing, as well as on the fourth and seventh days of growth. Taking pictures of the mycelium at various stages of development can provide valuable information on its growth rate, morphology, and behavior. On the final day of the experiment (7th day), the mycelium was scratched, and its wet weight was measured. Subsequently, it was placed in an oven for one hour at 80° C., following which its dry weight was determined. This process is commonly employed in microbiological and biochemical studies to assess the biomass of the sample, which can provide insights into its growth and metabolic activity. We will be evaluating the growth of other commercially important fungi (Table 3) other than Calocybe indica using micro- and macroalgae as substrate either alone or in combination with lignocellulosic biomass in the future.

TABLE 3
Commercially important order of fungal species.
Polyporales

| | |
|---|---|
| Ganoderma lucidum | Grifola frondosa |
| Fomes fomentarius | Daedaleopsis confragosa |

Agaricales

| | |
|---|---|
| Lentinula edodes | Pleurotus ostreatus |
| Calocybe Indica | Agrocybe aegerita |

Russulales
Hericium erinaceus
Pezizales
Morchella angusticeps

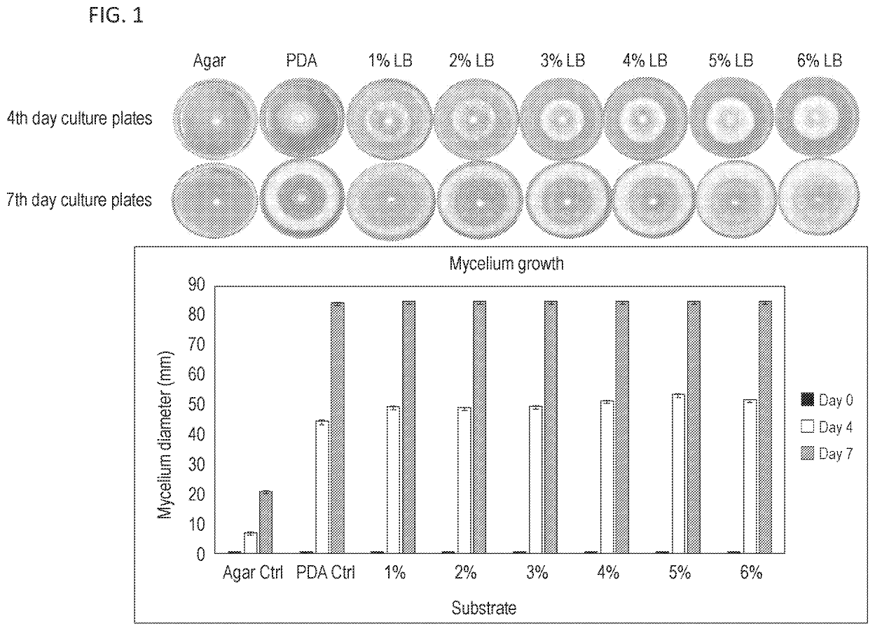

Results: Based on the findings, it can be inferred that the incorporation of algal biomass in the media has a positive impact on the development of mycelium. The data suggest that this type of media may provide the necessary nutrients and conditions for mycelial growth, leading to increased biomass. Overall, these results highlight the potential of algae substrate as a valuable component in the cultivation of mycelium. Further, research may be warranted to fully explore the benefits and optimal application of this approach. The results of culturing C. indica on lignocellulosic biomass Luria-Bertani (LB) Broth media are visually represented in FIG. 1, which depicts the mean diameter of mycelium observed across all samples. LB media was prepared at different concentrations ranging from 1% to 6% to optimize the growth conditions for Calocybe indica mycelium.

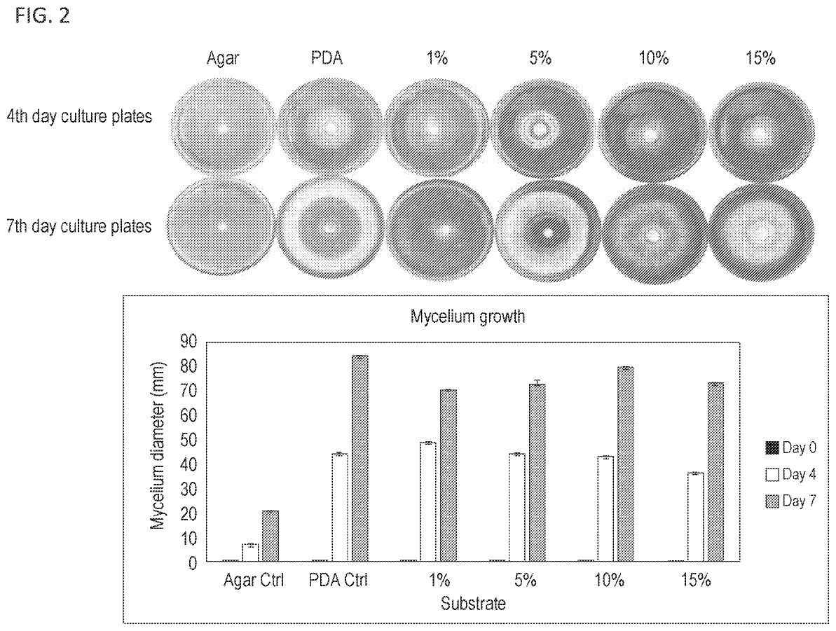

We evaluated microalgae such as Chlorella, Spirulina, and a combination of the two in a 1:1 ratio, were prepared with the aim of culturing Calocybe indica mycelium. The results of culturing Calocybe indica on chlorella-containing media are shown in FIG. 2, which depicts the mean diameter of mycelium observed across all samples. The results of our study indicate that the 5% concentration of chlorella algae media was the most effective in promoting mycelium growth in terms of mycelium growth and colony morphology.

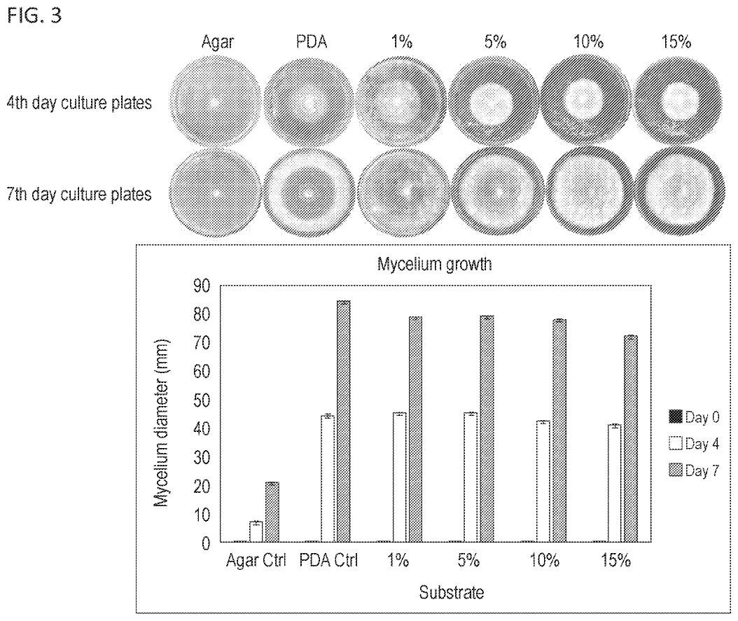

In another experimental design, Spirulina was used in the medium. FIG. 3 presents the results of the analysis of mycelium diameter and colony morphology of Calocybe indica when grown on Spirulina-containing media. It appears that the mycelium diameter of Calocybe indica increased as the concentration of Spirulina in the media increased from 1% to 5%. However, when the concentration of Spirulina in the media was further increased to 10% and 15%, the mycelium diameter decreased. This suggests that there is an optimal concentration of Spirulina in the media for the growth and development of Calocybe indica, and beyond this optimal concentration, the growth of Calocybe indica may be inhibited. The result shows that at the lower concentrations of Spirulina, Calocybe indica colonies appeared smaller, and their texture was dry and powdery. However, as the concentration of Spirulina increased, the colonies became larger, and their texture became moist and velvety.

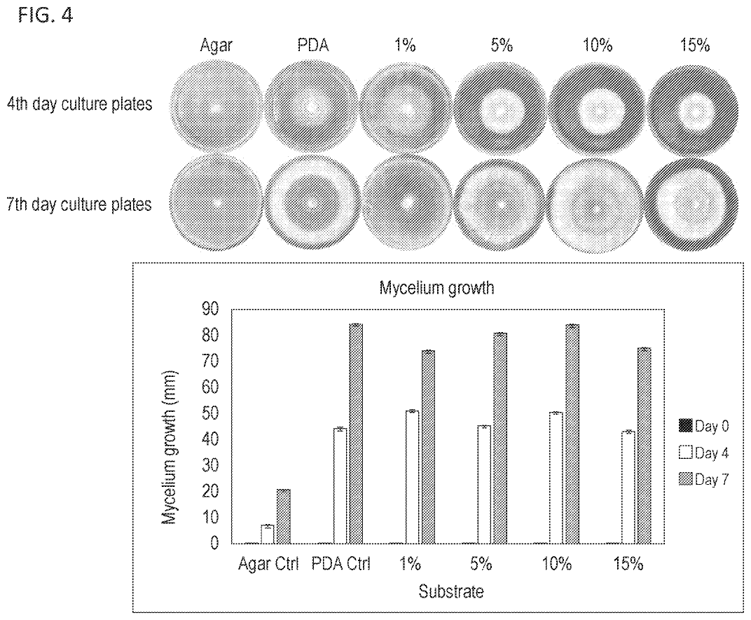

In another experiment, the combination of Spirulina and Chlorella at the ratio of 1:1 was used as the media for culturing Calocybe indica. FIG. 4 displays the mycelium diameter of Calocybe indica after being grown on a combination of Spirulina and Chlorella algae media for 7-days. The data indicates that the mycelium diameter increased as the concentration of algae in the media increased from 1% to 5% and 10%. However, when the concentration was increased from 10% to 15%, the mycelium diameter decreased.

Based on the result, increasing the concentration of algae in the media leads to better morphology compared to other concentrations. At 5% concentration, the morphology is comparable to the potato dextrose agar control, and at higher concentrations, it improves even further.

Our findings suggest that a 5% concentration of algae was determined to be the optimal concentration for promoting mycelial growth, as it resulted in similar or better results compared to PDA agar, which is a commonly used growth medium for fungi. Additionally, the cost-effectiveness of the 5% concentration was taken into consideration when determining the optimal concentration. It's possible that higher concentrations of algae may have resulted in even better growth of the mycelium compared to the 5% concentration.

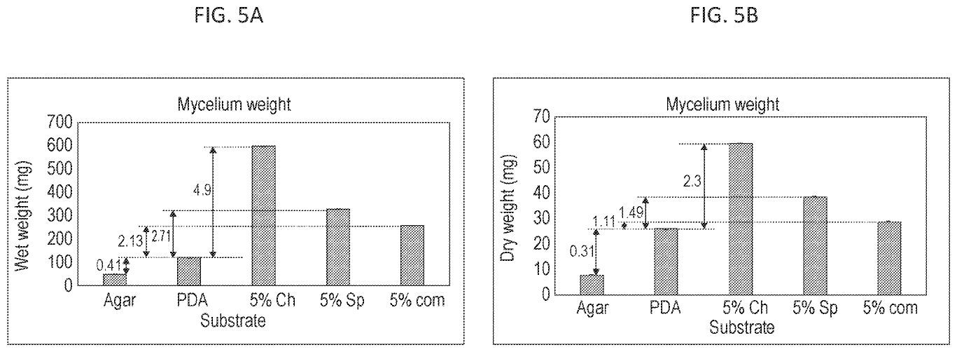

Moreover, the wet and dry weight of the mycelium grown on the 5% concentrations of algae in the media was measured. To determine the wet weight of the mycelium, the mycelium was scratched from the agar and weighed directly. To determine the dry weight of the mycelium, the mycelium was first weighed wet and then placed in an oven at 80° C. for one hour to remove all the moisture. After this process, the mycelium was weighed again to determine its dry weight. FIG. 5 shows the wet and dry weight of mycelium in the last day of the experiment.

The results suggest that 5% chlorella and a combination of two types of algae had similar results to using PDA (Potato Dextrose Agar), which is a commonly used growth medium for fungi. On the other hand, using 5% spirulina showed higher mycelium growth compared to using PDA.

In another experiment, 5% of spirulina, chlorella, and a combination of the two were added to the PDA agar to provide extra nutrients for mycelium growth. Using PDA agar and algae in combination may have better results compared to using agar and algae alone because PDA agar provides a nutrient-rich environment that supports the growth of many different types of fungi. When algae were added to PDA agar, it may act as an additional source of nutrients that can enhance the growth of fungi. In contrast, using only agar and algae may not provide sufficient nutrients to support optimal fungal growth. Agar is a solidifying agent that provides a surface for the fungi to grow on, but it does not contain the same range of nutrients as PDA agar. Algae, while a potential source of nutrients, may not provide all the necessary components for fungal growth, and may even inhibit fungal growth in some cases. By combining PDA agar with algae, it will be possible to create a more favorable environment for the growth of certain types of fungi, which could lead to better experimental outcomes. However, it is worth noting that the specific effects of combining PDA agar with algae may depend on the strains of fungi and algae being used, as well as other experimental variables such as temperature, pH, and nutrient availability.

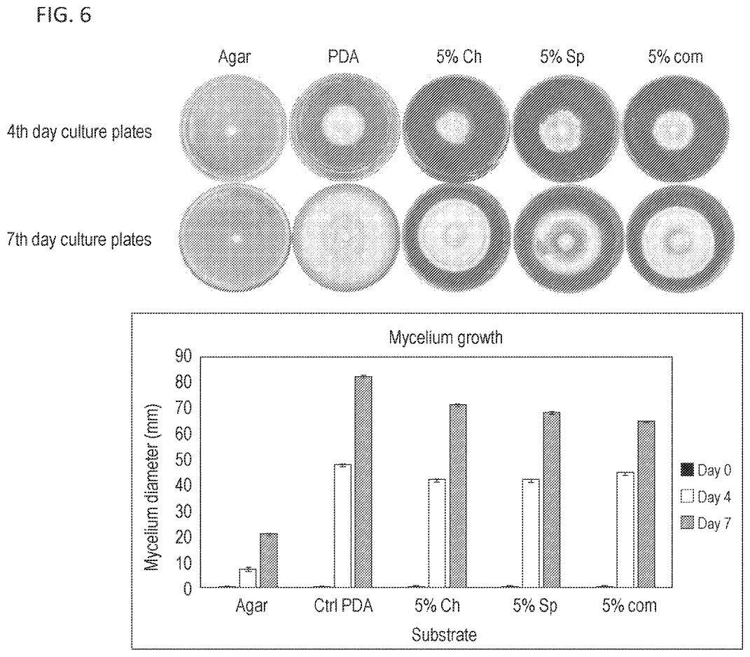

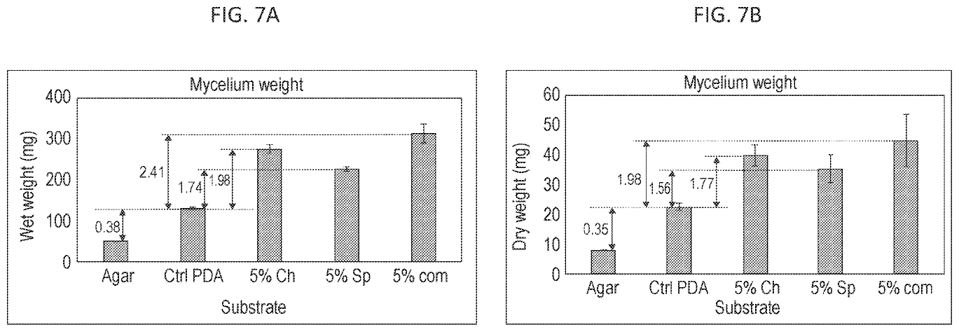

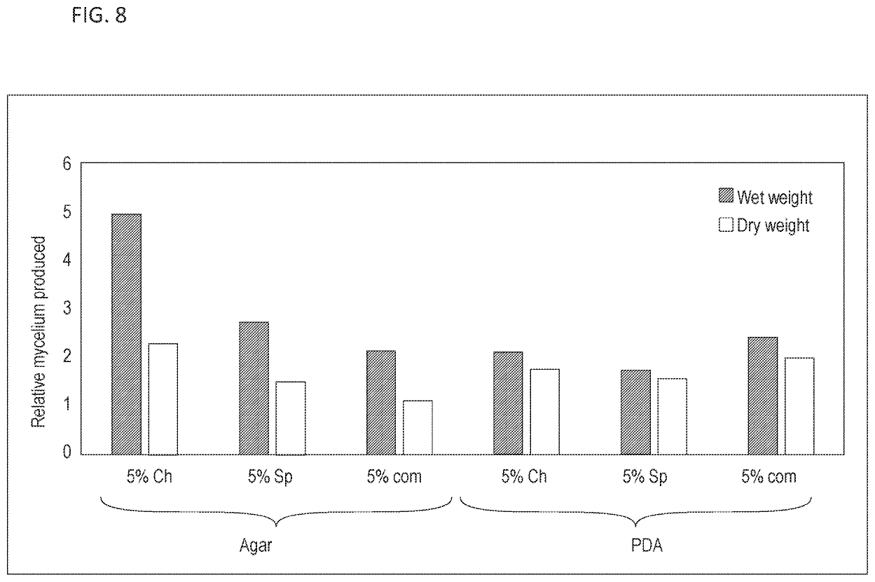

Based on FIG. 6 and FIG. 7, culturing mycelium on a combination of algae and PDA provided more growth nutrients than simple agar. This is evidenced by the significant improvement in mycelium weight despite the lower mycelium diameter when grown on algae-containing media. According to FIG. 8, the addition of 5% Chlorella resulted in a 5-fold increase in the wet weight of the mycelium, and the addition of a 5% combination of Chlorella and Spirulina with PDA resulted in a 2.5-fold improvement in the dry weight of the mycelium.

The results of the experiments suggest that algae can serve as a promising alternative nutrient source for mycelium culturing. When combined with agar, the algae proved to be effective in promoting mycelial growth, with better outcomes compared to the PDA control experiment. The increased mycelium weight observed in the algae and agar combination experiments is an indication of the potential of algae as a nutrient source for mycelial growth. Moreover, the positive results observed in the experiments could be attributed to the nutritional composition of algae. Algae are known to be rich in essential nutrients such as nitrogen, phosphorus, and potassium, which are crucial for the growth and development of mycelium. The presence of these nutrients in algae could have contributed to the enhanced mycelial growth observed in the experiments. The nutrients in the algae could have supplemented the limited nutrient content of the PDA medium, resulting in improved mycelial growth.

As part of the experiment, mycelium cultivation was facilitated by utilizing macroalgae as a source of nutrients. Macroalgae, also known as seaweed, is a type of aquatic plant that can grow to significant sizes in marine and freshwater environments. Macroalgae has numerous advantages and uses, making it a valuable resource for various industries. One advantage is its ability to absorb large amounts of CO₂, which can help mitigate the effects of climate change. Additionally, macroalgae is a rich source of nutrients and can be used as a food source for humans and animals. It is also used in the production of fertilizers, cosmetics, and biofuels. Furthermore, macroalgae can be cultivated in the ocean and does not require fresh water or arable land, making it a sustainable and environmentally friendly alternative to traditional crops.

Producing seaweed is cost-effective and ecologically sustainable since they contain abundant amounts of polysaccharides and other nutrients that nourish fungal mycelium to grow and develop. By utilizing these nutrients, mycelium can generate a range of enzymes and metabolites with diverse applications in various industries and medical fields. Furthermore, incorporating macroalgae as a nutrient source can reduce the reliance on expensive synthetic media, which may pose challenges in large-scale mycelium cultivation. Therefore, the use of macroalgae in mycelium cultivation has the potential to improve the efficiency and sustainability of fungal biotechnology.

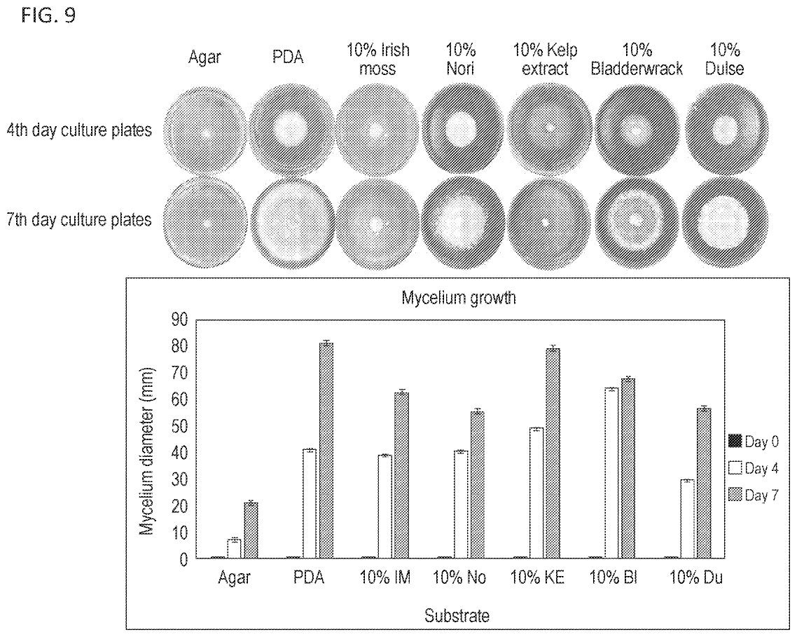

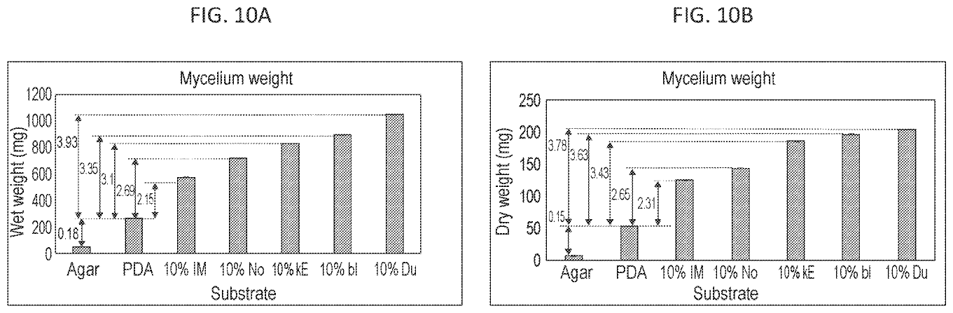

To promote mycelium growth, various macroalgae species such as dulse, Irish moss, bladderwrack, kelp extract, nori, and wakame were used. These macroalgae were added in a 10% (ww) concentration as a growth medium for the mycelium. To further investigate the effects of these macroalgae on mycelium growth, two different types of agar media were used: simple agar and potato dextrose agar (PDA). FIG. 9 and FIG. 10 depict the Calocybe indica mycelium diameter, wet and dry weight of Calocybe indica mycelium when exposed to a 10% concentration of the mentioned macroalgae.

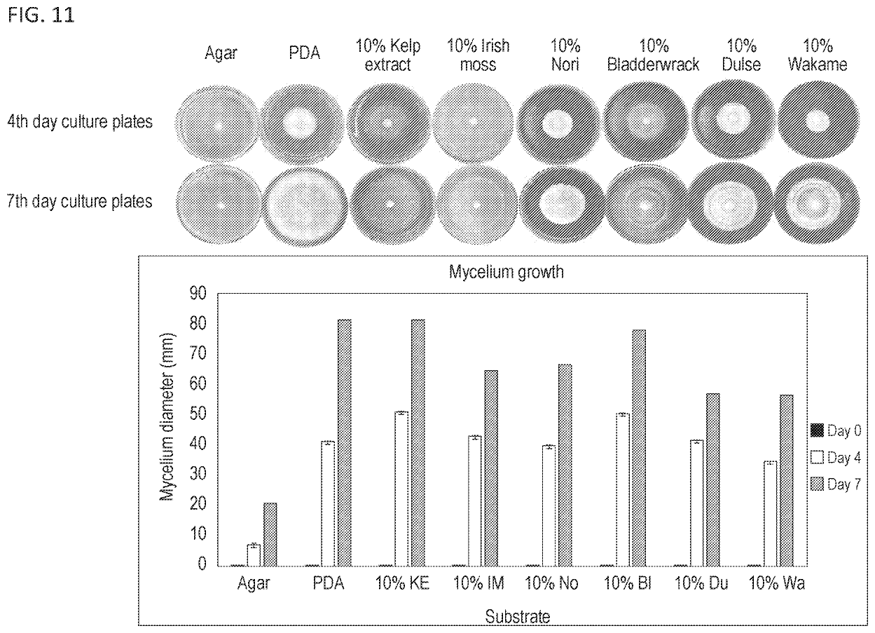

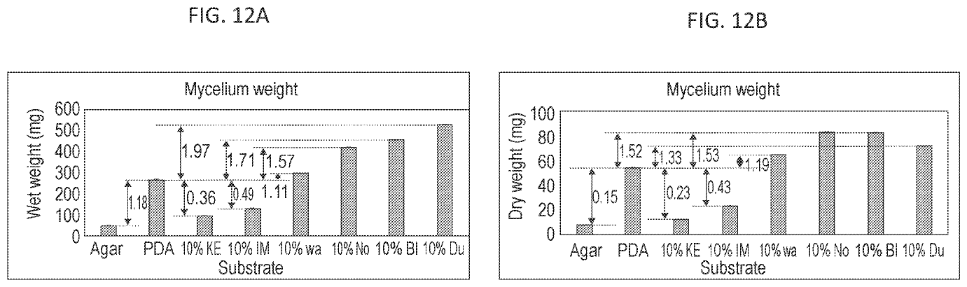

FIG. 11 and FIG. 12 depict the results of measuring the diameter, wet and dry weight of Calocybe indica mycelium on various types of macroalgae, including dulse, bladderwrack, nori, Irish moss, and kelp extract with agar.

On the final day of the experiment, measurements were taken of both the wet and dry weight of the mycelium. In another set of experiments, we tested the combination of lignocellulosic biomass and macroalgae at a ratio of 1:1 in a 10% concentration.

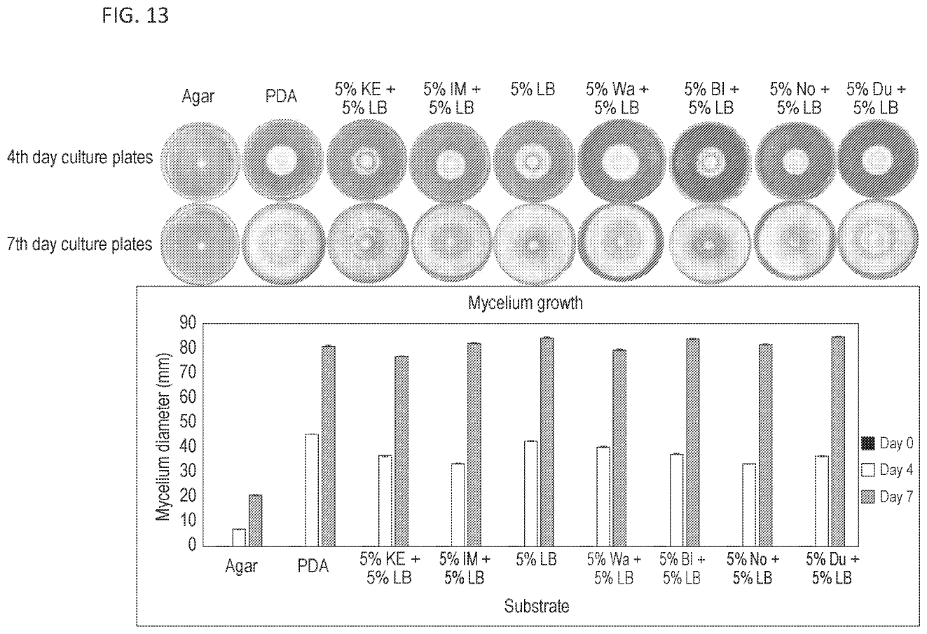

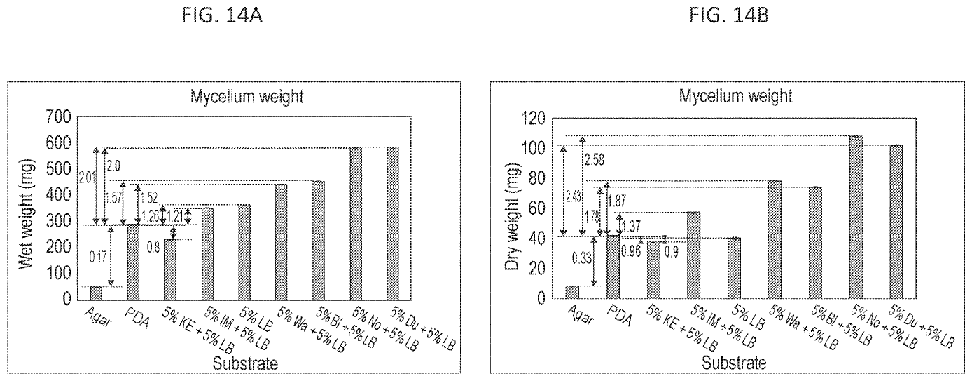

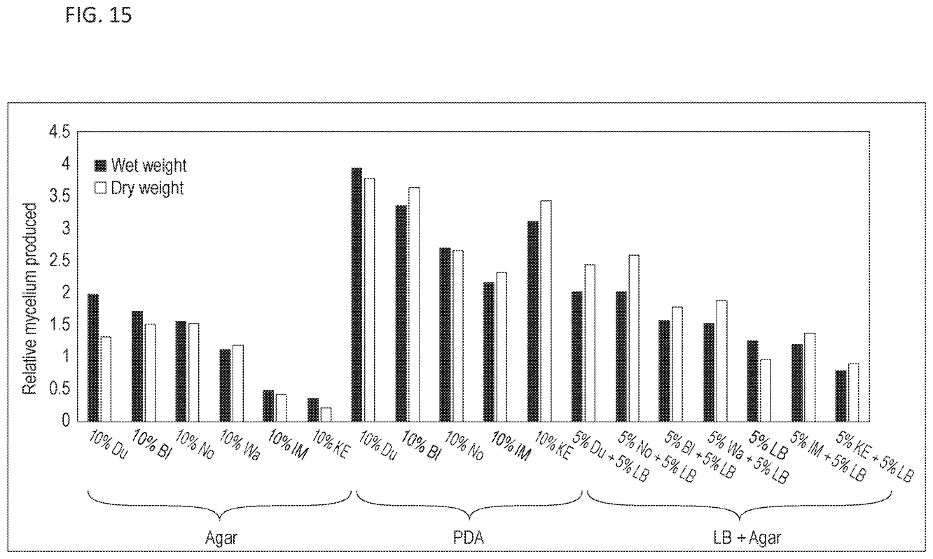

The results for culturing Calocybe indica on a combination of macroalgae and lignocellulosic biomass are displayed in FIG. 13 and FIG. 14. According to FIG. 15, the addition of 10% Dulse with agar and PDA resulted in a 2-fold and 4-fold improvement in the dry weight of the mycelium, respectively. Moreover, the combination of 5% Dulse with 5% lignocellulosic biomass and another combination of 5% Nori with 5% lignocellulosic biomass with agar resulted in around 2.5-fold improvement in the dry weight of the mycelium.

The results of our study indicate that the presence of both micro- and macroalgae can have a positive impact on mycelium growth. This is likely since these algae can serve as an additional source of nutrients for the mycelium, which in turn promotes its growth. While there were some cases in which the mycelium diameter was smaller compared to the control group grown on PDA media, the overall weight of the mycelium was greater in the presence of algae. This increase in weight can be attributed to the formation of a mycelium mat, which provides a larger surface area for nutrient absorption and ultimately leads to greater mycelium growth.

FIG. 13 and FIG. 14 clearly show that the combination of lignocellulosic biomass and macroalgae can significantly impact both mycelium growth diameter and weight. This is a promising finding for the mushroom farming industry, as it suggests that using a combination of these materials could lead to a more efficient and environmentally sustainable process. Lignocellulosic biomass is a byproduct of various industries, such as agriculture and forestry, and is typically considered as a waste material. By utilizing this material in conjunction with macroalgae as a nutrient source for mycelium growth, we can reduce waste and create a more circular approach to agriculture. Additionally, the increased mycelium growth and weight observed in the presence of both materials suggests that this combination could lead to faster mycelium growth and producing mushrooms in a shorter period. This will be beneficial for commercial mushroom growers. Overall, our study provides promising evidence for the use of a combination of lignocellulosic biomass and algae in mushroom farming as a means of improving efficiency and reducing environmental impact. While our study specifically focused on the impact of micro- and macroalgae on the growth of Calocybe indica mycelium, it is possible that similar results could be observed for other commercially important fungus such as Oyster mushrooms and Ganoderma (as given in Table 3). These mushrooms have different nutritional requirements and growth patterns, so further research would be necessary to determine the optimal combination and concentration of lignocellulosic biomass and algae for these species.

### Additional Work

Method and material: This study was conducted in two phases to investigate the potential of using algae-based substrates for mushroom cultivation. In the first phase, mycelium growth on algae-based substrates was evaluated in Petri dishes under controlled laboratory conditions. The second phase focused on fruiting body production using the best-performing algae-based substrates from phase one, which were tested in mushroom cultivation bottles. Both phases used four mushroom species: Hypsizygus ulmarius, Calocybe indica, Pleurotus ostreatus, and Ganoderma lucidum.

Petridis mycelium production method: Macroalgae (dulse, and nori) and microalgae (Chlorella and Spirulina) powders were mixed with lignocellulosic biomass (wheat straw, wheat bran and corn flour) at 10% ratio dry basis. The mixtures were filled into 90 mm Petri dishes (20 g per dish) and sterilized by autoclaving at 121° C. for 30 minutes. The sterilized Petri dishes were inoculated with a 1 cm² piece of actively growing mycelium from pure cultures of the four mushroom species and incubated at 25° C. in the dark. On the last day of the experiment, mycelium was harvested and put into the oven at 80° C., over overnight, and dry weight was weighed.

To study the effect of salt on mycelium growth in a petri dish, macroalgae, and microalgae powder were mixed with water, centrifuged, and the supernatant was separated. The algae were then used for cultivation in petri dishes. However, because the concentration of algae was only 10%, the results for mycelium growth with and without salt removal did not show a significant difference.

Producing mushroom: Based on the Petridis experiments, the best-performing algae-based substrates for each fungal species were selected for producing fruiting body. The lignocellulosic biomass consisted of wheat straw (85%), wheat bran (10%), corn flour (5%), gypsum (1%), and lime (0.2%). Two substrate formulations were prepared: (1) 100% lignocellulosic biomass and (2) 50% lignocellulosic biomass+50% algae. The moisture content was adjusted to 70%, and the substrates were filled into polypropylene bottles (500 g per bottle), sealed with a plastic cap and a cotton plug, and sterilized by autoclaving at 121° C. for 30 minutes. The sterilized bottles were inoculated with a 5% (w/w) grain spawn of the respective mushroom species and incubated at 20° C. in the dark until complete mycelium colonization was achieved. The bottles were then transferred to a fruiting room maintained at 18-20° C., 85-90% relative humidity, and 500-1000 lux light intensity for 12 hours daily. Plastic caps were removed, and cotton plugs were loosened to initiate fruiting body formation. Fruiting bodies were harvested at maturity, and each bottle's fresh weight was recorded. All experiments were conducted in triplicate, and data were expressed as mean±standard deviation.

Pretreatment of Macroalgae: Using macroalgae as a substrate for mushroom cultivation presented two main challenges: high salt content and large particle size. In the first set of experiments, the mycelium could not grow on the actual substrate due to the high salt concentration. The macroalgae were pretreated before being incorporated into the substrate formulations to address these issues.

Salt removal: Macroalgae, being marine organisms, naturally contain high levels of salt, which can inhibit the growth and development of fungi. The macroalgae were soaked in fresh water to reduce the salt content for 2 hours. During this soaking period, the salt from the macroalgae diffused into the water, effectively lowering the salt concentration in the algal biomass. After soaking, a spinner drained the macroalgae and gently spun to remove excess water. This simple yet effective pretreatment method ensured that the salt content in the macroalgae was reduced to a level suitable for mushroom cultivation.

Size reduction: The large particle size of the macroalgae posed another challenge for their use as a substrate. Large particles can hinder the colonization of the substrate by the mycelium, leading to slower growth rates and reduced substrate utilization. To overcome this issue, the pretreated macroalgae were chopped into smaller pieces using a mechanical chopper. The target particle size was approximately 1-2 cm, which is like the size of the lignocellulosic biomass used in the study. The size reduction of the macroalgae increased their surface area, making them more accessible for the fungus and facilitating a more uniform distribution of the algal biomass within the substrate.

In some embodiments, these pretreatment methods addressed the macroalgae's high salt content and large particle size, rendering them more suitable for use as a substrate in mushroom cultivation. In some embodiments, the salt removal and size reduction processes were important steps in optimizing the performance of the algae-based substrates and ensuring the successful growth and development of the studied mushroom species.

The results of this study demonstrated that the use of algae-based substrates, either alone or in combination with lignocellulosic biomass, significantly influenced the mycelium growth and fruiting body production of the five studied mushroom species: Hypsizygus ulmarius, Calocybe indica, Pleurotus ostreatus, Ganoderma lucidum and Agaricus bisporus (button mushroom). Additionally, the cultivation of ELM mushrooms on algae-based substrates while producing mushrooms showed remarkable improvements in biological efficiency. The following sections will present the detailed results for each fungal species grown on different substrates, including lignocellulosic biomass, algae alone, and their combinations.

Hypsizygus ulmarius (ELM oyster mushroom): The results obtained for Hypsizygus ulmarius reveal that using macroalgae as a substrate leads to a more than 2-fold increase in mycelium growth compared to the lignocellulosic biomass (FIG. 16). This finding highlights the superior performance of macroalgae as an alternative substrate for cultivating Hypsizygus ulmarius mycelium, demonstrating its potential to significantly enhance the efficiency and productivity of mushroom cultivation processes. In addition to macroalgae, the study also investigated using microalgae as substrates for Hypsizygus ulmarius mycelium growth.

The results showed that microalgae could also enhance mycelium growth compared to lignocellulosic biomass, although the improvement was not as substantial as that observed with macroalgae substrates. This finding suggests that both macroalgae and microalgae have the potential to serve as effective substrates for Hypsizygus ulmarius cultivation, with macroalgae showing superior performance. Combining chlorella with lignocellulosic biomass showed the highest mycelium growth.

Calocybe indica (Milky white mushrooms): The Calocybe indica results demonstrate that using macroalgae, specifically Nori and Dulse, as substrates can significantly enhance mycelium growth compared to lignocellulosic biomass (FIG. 17). The experiments showed a remarkable 5.4-fold increase in mycelium growth when using Nori and a 4.9-fold increase when using Dulse as substrates, compared to the growth observed on lignocellulosic biomass alone. Furthermore, even when Nori and Dulse were mixed with lignocellulosic biomass, the mycelium growth of Calocybe indica was notably improved compared using lignocellulosic biomass alone.

The study also investigated using microalgae as substrates for Calocybe indica mycelium growth. While the results showed that microalgae could indeed enhance mycelium growth compared to lignocellulosic biomass, the improvement was not as substantial as that observed with macroalgae substrates. This difference in performance may be attributed to the distinct nutritional profiles and cell wall compositions of macroalgae and microalgae that influence the Calocybe indica mycelium growth.

Pleurotus ostreatus (Oyster Mushroom): The study found that microalgae substrates, such as Chlorella and Spirulina, performed significantly better than lignocellulosic biomass in supporting the mycelium growth of Pleurotus ostreatus. The superior performance of microalgae may be due to their high protein content, balanced amino acid composition, and the presence of various growth-promoting compounds, such as vitamins and minerals. The small size and relatively simple cell wall structure of microalgae may also contribute to improved mycelium growth by facilitating easier nutrient uptake and digestion.

However, contrary to the previous statement, the macroalgae substrates, specifically Dulse and Nori, did not show a significant improvement in mycelium growth compared to lignocellulosic biomass for Pleurotus ostreatus. This suggests that not all macroalgae species may be equally effective in enhancing mycelium growth for this mushroom species (FIG. 18).

Interestingly, when microalgae were combined with lignocellulosic biomass, the mycelium growth of Pleurotus ostreatus was significantly enhanced, showing up to a 2-fold increase compared to the growth of lignocellulosic biomass alone. This synergistic effect suggests that microalgae can complement and improve the nutritional profile of lignocellulosic substrates, providing additional nutrients and growth-promoting factors that support the robust development of mycelium.

Furthermore, combining macroalgae and microalgae with lignocellulosic biomass also improved mycelium growth compared to lignocellulosic biomass alone. Although the macroalgae substrates alone may not have significantly improved, their incorporation alongside microalgae and lignocellulosic biomass positively impacts mycelium growth.

Ganoderma lucidum (Reishi Mushroom): The results obtained for Ganoderma lucidum showed new insights into the effects of using algae as substrates for mycelium growth. The study found that when macroalgae and microalgae were used alone as substrates, they did not significantly enhance the mycelium growth of Ganoderma lucidum compared to lignocellulosic biomass (FIG. 19).

However, a remarkable synergistic effect was observed when macroalgae and microalgae were combined with lignocellulosic biomass. The mycelium growth of Ganoderma lucidum was significantly enhanced, showing up to a 5-fold increase compared to the development of lignocellulosic biomass alone. This finding suggests that incorporating algae into traditional lignocellulosic substrates can dramatically improve the overall performance of the substrate for mycelium cultivation of Reishi mushrooms.

Agaricus bisporus (button mushrooms): The results for the Agaricus bisporus cultivation in phase one highlighted the effectiveness of using Chlorella and Dulse as substrates, either alone or in combination with composted lignocellulosic biomass. When Chlorella and Dulse were used as the sole substrate components, they significantly enhanced the growth and biological efficiency of Agaricus bisporus compared to the control substrate, which consisted of 100% lignocellulosic biomass. The mushrooms grown on substrates containing only Chlorella or Dulse exhibited a remarkable 2-fold increase in growth and biological efficiency (FIG. 20).

The most impressive result, however, was observed when composted lignocellulosic biomass was combined with Dulse in a 50:50 ratio. This substrate formulation led to an outstanding 3-fold increase in the biological efficiency of Agaricus bisporus compared to the control. The synergistic effect between the composted lignocellulosic biomass and Dulse can be attributed to several factors.

In another experiment, inoculation on actual substrates was conducted. ELM oyster mushroom (Hypsizygus ulmarius) was cultivated on lignocellulosic biomass and algae in this experiment. The first set of experiments involved using different types of macroalgae, such as sea moss, dulse, nori, kombu kelp, and sea lettuce. The results showed that certain kinds of macroalgae, like dulse and nori, were unsuitable for this experiment because they are soft and do not allow for proper air regulation. However, other types, such as sea moss and sea lettuce, proved to be suitable substrates. The experiments were conducted using pure macroalgae substrates or combined with lignocellulosic biomass. The results (FIG. 21) demonstrated that pure macroalgae substrates yielded the best outcomes.

After just 10 days, the pure macroalgae substrates were fully colonized, and the mushroom fruiting bodies started to appear, achieving a biological efficiency of 100%. In contrast, combining macroalgae and lignocellulosic biomass took two weeks to fully colonize. Lignocellulosic biomass alone required a significantly longer time, taking 4 to 6 weeks for complete colonization and initiating fruiting body formation. These findings highlight the potential of using pure macroalgae substrates, particularly sea moss and sea lettuce, to efficiently cultivate elm oyster mushrooms. The rapid colonization and high biological efficiency observed with pure macroalgae substrates demonstrate their superiority compared to the combination of macroalgae and lignocellulosic biomass or lignocellulosic biomass alone.

The technology described here presents a closed-loop system that integrates mushroom/mycelium cultivation, CO₂ capture, and algal biomass production, creating a sustainable and eco-friendly approach to mushroom production (FIG. 22B). The system operates as follows:

CO₂ capture from mushroom/mycelium chambers: The CO₂ generated during the mushroom/mycelium cultivation process is captured from the growth chambers. This step prevents the direct release of CO₂ into the atmosphere, reducing the cultivation process's carbon footprint.

Conversion of CO₂ to NaHCO₃: The captured CO₂ is then converted into sodium bicarbonate (NaHCO₃) through a chemical process. This conversion efficiently utilizes the captured CO₂ and provides a carbon source for subsequent production.

Transfer of NaHCO₃ to algal biomass system: The produced NaHCO₃ is transferred to the algal biomass production system. Sodium bicarbonate serves as a carbon source for the growth and development of algae, promoting efficient algal biomass production.

Algal biomass production: The algal biomass system utilizes the NaHCO₃ derived from the captured CO₂ to cultivate algae. Algae have a high growth rate and can efficiently convert CO₂ and carbon sources into biomass, effectively sequestering carbon and producing a valuable substrate for mushroom/mycelium cultivation.

Utilization of algal biomass as a substrate for mushroom/mycelium production: The produced algal biomass is then used to cultivate mushrooms and mycelium. As demonstrated in the previous experiments, algal biomass has shown promising results as a substrate, enhancing mycelium growth, reducing cultivation time, and improving biological efficiency compared to traditional lignocellulosic biomass substrates.

Utilization of spent mushroom substrate as animal feed: After the mushroom/mycelium cultivation process, the spent substrate, which is rich in nutrients, is utilized as animal feed. This step further maximizes the system's resource efficiency by providing a valuable by-product for animal nutrition.

The closed-loop system described here offers several advantages:
Sustainability: The system reduces mushroom production's carbon footprint by capturing and utilizing CO₂ from the mushroom/mycelium cultivation process. The conversion of CO₂ to NaHCO₃ and its subsequent use in algal biomass production creates a sustainable cycle that mitigates greenhouse gas emissions.
Resource efficiency: Integrating mushroom/mycelium cultivation, CO₂ capture, and algal biomass production optimizes resource utilization. The system efficiently recycles CO₂, carbon, and nutrients, minimizing waste and maximizing the productivity of each component.
Enhanced mushroom production: The use of algal biomass as a substrate for mushroom/mycelium cultivation has shown promising results in terms of improved growth, reduced cultivation time, and increased biological efficiency. This enhancement in mushroom production efficiency can lead to higher yields and reduced operational costs.
Valorization of by-products: Using spent mushroom substrate as animal feed adds value to the by-products of the mushroom cultivation process. This approach promotes a circular economy, where waste is minimized and resources are maximized.

The closed-loop system presented here showcases a holistic approach to sustainable mushroom production, integrating CO₂ capture, algal biomass production, and the utilization of by-products. This technology can potentially revolutionize the mushroom industry, promoting eco-friendly practices, reducing environmental impact, and enhancing the efficiency and profitability of mushroom cultivation.

The use of pure macroalgae substrates not only reduces the cultivation time but also eliminates the need for lignocellulosic biomass, which is associated with longer colonization periods. This approach can lead to more efficient and sustainable mushroom production, as it minimizes the time and resources required for substrate preparation and fruiting body formation.

In conclusion, the use of macroalgae as a substrate for the cultivation of various mushroom species, including elm oyster mushroom (Hypsizygus ulmarius), milky white mushroom (Calocybe indica), oyster mushroom (Pleurotus ostreatus), and reishi mushroom (Ganoderma lucidum), has demonstrated remarkable potential in enhancing the efficiency and sustainability of mushroom production. The experiments conducted with pure macroalgae substrates and their combinations with lignocellulosic biomass have shown promising results across all the studied mushroom species.

1. Reduced cultivation time: Pure macroalgae substrates and their combinations with lignocellulosic biomass have shown faster colonization and fruiting body formation than lignocellulosic biomass alone. This reduction in cultivation time was observed across all the studied mushroom species, with some variations depending on the specific macroalgae used.

2. Enhanced biological efficiency: Using macroalgae substrates has resulted in higher biological efficiencies, indicating improved substrate utilization and increased mushroom yield. This enhancement was particularly evident in the case of Calocybe indica, where a 5.4-fold and 4.9-fold increase in mycelium growth was observed on Nori and Dulse substrates, respectively.

3. Energy and cost savings: The shorter cultivation times associated with macroalgae substrates translate to reduced energy consumption and operational costs. This benefit applies to all the studied mushroom species, as less time and resources are required for substrate preparation and maintenance.

4. Reduced CO₂ emissions: By incorporating macroalgae into the substrate, either alone or in combination with lignocellulosic biomass, the CO₂ emissions associated with mushroom production can be mitigated. Macroalgae could absorb CO₂ during their growth, offsetting the emissions generated during cultivation.

The findings of this study highlight the immense potential of macroalgae as a sustainable and efficient substrate for cultivating various mushroom species. Adopting this approach on a larger scale could revolutionize the mushroom industry, leading to more environmentally friendly and cost-effective production methods across different mushroom varieties. Further research should focus on optimizing the cultivation conditions for each mushroom species, exploring the potential of other macroalgae species, and investigating the nutritional and functional properties of mushrooms grown on these substrates. Additionally, assessing the scalability and economic viability of using macroalgae substrates in commercial mushroom production would provide valuable insights for the industry.

In some embodiments, the mushroom is not a Cordyceps. In some embodiments, the mushroom is not Cordyceps militaris. In some embodiments, the mushroom is not of the genus Cordyceps.

In some embodiments, the fungus is not a Cordyceps. In some embodiments, the fungus is not Cordyceps militaris. In some embodiments, the fungus is not of the genus Cordyceps.

In some embodiments, the mycelium is not from a Cordyceps. In some embodiments, the mycelium is not from Cordyceps militaris. In some embodiments, the mycelium is not from the genus Cordyceps.

In some embodiments, the algae is not a Spirulina. In some embodiments, the algae is not Spirulina maxima. In some embodiments, the algae is not of the genus Spirulina.

In some embodiments, the microalgae is not a Spirulina. In some embodiments, the microalgae is not Spirulina maxima. In some embodiments, the microalgae is not of the genus Spirulina.

In some embodiments, the algae is not an Arthrospira. In some embodiments, the algae is not Arthrospira maxima. In some embodiments, the algae is not of the genus Arthrospira.

In some embodiments, the microalgae is not an Arthrospira. In some embodiments, the microalgae is not Arthrospira maxima. In some embodiments, the microalgae is not of the genus Arthrospira.

In some embodiments, the algae is not a Chlorella. In some embodiments, the algae is not Chlorella vulgaris. In some embodiments, the algae is not of the genus Chlorella.

In some embodiments, the microalgae is not a Chlorella. In some embodiments, the microalgae is not Chlorella vulgaris. In some embodiments, the microalgae is not of the genus Chlorella.

The various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

It is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention.

1. A substrate for cultivating at least one fungal mycelium or at least one mushroom, the substrate comprising: at least one algae, provided that the at least one algae is not Spirulina maxima or Chlorella vulgaris.

2. The substrate of claim 1, wherein the at least one algae has been treated to remove at least a portion of salt from the at least one algae.

3. The substrate of claim 1, wherein the substrate further comprises lignocellulosic biomass.

4. The substrate of claim 1, wherein the at least one algae is at least one microalgae, or at least one macroalgae, or combination thereof, provided that the at least one microalgae is not Spirulina maxima or Chlorella vulgaris.

5. The substrate of claim 4, wherein the at least one macroalgae is nori, bladderwrack, Irish moss, kelp, dulse, or wakame, or any combination thereof.

6. The substrate of claim 1, wherein the at least one fungal mycelium or the at least one mushroom is Hypsizygus ulmarius, Calocybe indica, Pleurotus ostreatus, Ganoderma lucidum, Agaricus bisporus, Grifola frondosa, Fomes fomentarius, Daedaleopsis confragosa, Lentinula edodes, Agrocybe aegerita, Hericium erinaceus, or Morchella angusticeps, or any combination thereof.

7. A method for cultivating at least one fungal mycelium or at least one mushroom, the method comprising:
providing a substrate of claim 1;
inoculating the substrate with fungal spores or fungal mycelium; and
applying conditions to stimulate growth of at least one fungal mycelium or at least one mushroom.

8. The method of claim 7, wherein the substrate further comprises lignocellulosic biomass.

9. The method of claim 7, wherein the at least one algae is at least one microalgae, or at least one macroalgae, or combination thereof, provided that the at least one microalgae is not Spirulina maxima or Chlorella vulgaris.

10. The method of claim 9, wherein the at least one macroalgae is nor, bladderwrack, Irish moss, kelp, dulse, or wakame, or any combination thereof.

11. A method for cultivating at least one fungal mycelium or at least one mushroom and for producing algal biomass, the method comprising:
providing a substrate of claim 1;
inoculating the substrate with fungal spores or fungal mycelium;
applying conditions to stimulate growth of at least one fungal mycelium or at least one mushroom;
capturing carbon dioxide produced during the cultivation of the at least one fungal mycelium or the at least one mushroom;
converting the captured carbon dioxide into sodium bicarbonate; and
utilizing the sodium bicarbonate as a carbon source to produce algal biomass.

12. The method of claim 11, wherein the substrate further comprises lignocellulosic biomass.

13. The method of claim 11, wherein the at least one algae is at least one microalgae, or at least one macroalgae, or combination thereof, provided that the at least one microalgae is not Spirulina maxima or Chlorella vulgaris.

14. The method of claim 13, wherein the at least one macroalgae is non, bladderwrack, Irish moss, kelp, dulse, or wakame, or any combination thereof.

15. A system for cultivating at least one fungal mycelium or at least one mushroom and for producing algal biomass, the system comprising:
a first chamber, wherein the first chamber is adapted for cultivating at least one fungal mycelium or at least one mushroom and capturing carbon dioxide produced during cultivation of the at least one fungal mycelium or the at least one mushroom, and wherein the first chamber comprises a substrate of claim 1;
a second chamber, wherein the second chamber is in communication with the first chamber, and wherein the second chamber is adapted to convert the captured carbon dioxide into sodium bicarbonate; and
a third chamber, wherein the third chamber is in communication with the second chamber, and wherein the third chamber is adapted for using the sodium bicarbonate as a carbon source for producing algal biomass.

16. The system of claim 15, wherein the substrate further comprises lignocellulosic biomass.

17. The system of claim 15, wherein the at least one algae is at least one microalgae, or at least one macroalgae, or combination thereof, provided that the at least one microalgae is not Spirulina maxima or Chlorella vulgaris.

18. The system of claim 17, wherein the at least one macroalgae is nor, bladderwrack, Irish moss, kelp, dulse, or wakame, or any combination thereof.

19. The system of claim 15, wherein the at least one mushroom is Hypsizygus ulmarius, Calocybe indica, Pleurotus ostreatus, Ganoderma lucidum, Agaricus bisporus, Grifola frondosa, Fomes fomentarius, Daedaleopsis confragosa, Lentinula edodes, Agrocybe aegerita, Hericium erinaceus, or Morchella angusticeps, or any combination thereof.

20. The system of claim 15, wherein the system further comprises a fourth chamber, wherein the fourth chamber is in communication with the third chamber, and wherein the fourth chamber is adapted for storing algal biomass.
